(19) United States
(12) Patent Application Publication
(10) Pub. No.: US 2018/0265887 A1
(43) Pub. Date: Sep. 20, 2018

(54) Basil Plants With High Tolerance to Downy Mildew

(71) Applicants: Jacobs Farm Del Cabo, Pescadero, CA (US); Agroproductos Del Cabo S.A. de C.V., Santa Rosa (MX)

(72) Inventors: Ricardo Magaña Acosta, Santa Rosa (MX); Marbella Vanessa Martiñon Bautista, Buenos Aires (MX); Robert Osteen Pierce, Watsonville, CA (US)

(73) Assignees: Jacobs Farm Del Cabo, Pescadero, CA (US); Agroproductos Del Cabo S.A. de C.V., Santa Rosa (MX)

(21) Appl. No.: 15/923,717

(22) Filed: Mar. 16, 2018

Related U.S. Application Data
(60) Provisional application No. 62/472,103, filed on Mar. 16, 2017.

(51) Int. Cl. C12N 15/82; A01H 5/12; C12N 5/04; A01H 4/00; A01H 1/02; C12Q 1/6895; A01H 1/08; A01H 1/06

(52) U.S. Cl. CPC C12N 15/8282; A01H 5/12; C12N 5/04; A01H 4/005; C12Q 2600/13; C12Q 1/6895; A01H 1/08; A01H 1/06; A01H 1/02

(57) ABSTRACT

Ocimum basilicum plants or germplasm having tolerance or improved tolerance to Peronospora belbahrii infection, comprising in its genome the single nucleotide polymorphisms (SNPs) of SEQ ID NOs:1-630 or having approximately 90% to 0.5% of said SNPs in said Ocimum basilicum genome are disclosed. Further embodiments include methods of producing, selecting, and evaluating the responses of Ocimum basilicum plants or germplasm having tolerance or improved tolerance to Peronospora belbahrii infection; said plants comprised of at least one dominant allele and at least one recessive allele in the homozygous state that produce higher levels of tolerance to Peronospora belbahrii; seeds, plants, plant parts, methods for producing basil plants by crossing said basil plant with itself or with another basil plant, methods for producing sweet basil plants having higher tolerance to Peronospora belbahrii, plants and plant parts produced by those methods and methods for producing interspecific basil plants having tolerance to Peronospora belbahrii.

### CROSS REFERENCE TO RELATED APPLICATION

This application is a non-provisional patent application of and claims the benefit of priority to U.S. Provisional Application No. 62/472,103 filed on Mar. 16, 2017, the entire contents of which are incorporated herein by reference for all purposes.

### SUBMISSION OF SEQUENCE LISTING

The Sequence Listing associated with this application is filed in electronic format via EFS-Web and is hereby incorporated by reference into the specification in its entirety

### BACKGROUND

All publications cited in this application are herein incorporated by reference.

Ocimum basilicum, commonly known as sweet basil, is an herb from the genus of Laminaceae. Basil is typically propagated from seed or tip cuttings but can also be propagated from tissue culture. Seed, cuttings and tissue culture germination protocols for basil are well-known in the art.

Basil is an important and valuable culinary plant that is widely used in food all over the world. Although basil is most commonly grown as an annual, in warmer climates, basil can be grown as a perennial.

The foregoing examples of the related art and limitations related therewith are intended to be illustrative and not exclusive. Other limitations of the related art will become apparent to those of skill in the art upon a reading of the specification.

### SUMMARY

The following embodiments and aspects thereof are described in conjunction with systems, tools and methods which are meant to be exemplary, not limiting in scope. In various embodiments, one or more of the above-described problems have been reduced or eliminated, while other embodiments are directed to other improvements.

One embodiment provides for an Ocimum basilicum plant or germplasm that exhibits tolerance or improved tolerance to Peronospora belbahrii infection, comprising in its genome the single nucleotide polymorphisms (SNPs) of SEQ ID NOs:1-630. Other embodiments provide for having approximately 90%, 90%, 70%, 60%, 50%, 40%, 30%, 20%, 10%, 5%, and 0.5% of the single nucleotide polymorphisms (SNPs) of SEQ ID NOs:1-630 in the Ocimum basilicum genome.

Another embodiment provides for a method of producing an Ocimum basilicum plant or germplasm that exhibits tolerance or improved tolerance to Peronospora belbahrii infection, the method comprising: isolating nucleic acids from a genome of a first Ocimum basilicum plant or germplasm; detecting in the first Ocimum basilicum plant or germplasm at least one or more single nucleotide polymorphisms (SNPs) of SEQ ID NOs:1-630 that is associated with the tolerance or improved tolerance to Peronospora belbahrii infection; selecting said first Ocimum basilicum plant or germplasm, or selecting a progeny of said first Ocimum basilicum plant or germplasm wherein the plant, germplasm or progeny comprises at least one or more single nucleotide polymorphisms (SNPs) of SEQ ID NOs:1-630 associated with tolerance or improved tolerance to Peronospora belbahrii infection; and introgressing the at least one or more single nucleotide polymorphisms (SNPs) of SEQ ID NOs:1-630 associated with tolerance or improved tolerance to Peronospora belbahrii infection of the first Ocimum basilicum plant or germplasm into a second Ocimum basilicum plant or germplasm to produce an introgressed Ocimum basilicum plant or germplasm. Another embodiment provides for an Ocimum basilicum plant or germplasm that exhibits tolerance or improved tolerance to Peronospora belbahrii produced by said method.

Another embodiment provides for a method of selecting an Ocimum basilicum plant or germplasm that exhibits tolerance or improved tolerance to Peronospora belbahrii infection, the method comprising: detecting in an Ocimum basilicum plant one or more loci associated with a polynucleotide, wherein the polynucleotide discriminates between an Ocimum basilicum that exhibits tolerance or improved tolerance and an Ocimum basilicum that exhibits susceptibility to Peronospora belbahrii infection, and wherein the polynucleotide detects at least one or more SNP within a genomic sequence set forth in SEQ ID NOs:1-630; and selecting the Ocimum basilicum plant or germplasm comprising the detected one or more loci, thereby selecting an Ocimum basilicum plant with tolerance or improved tolerance to Peronospora belbahrii infection.

According to another embodiment, there are provided basil plants comprising a highly heritable trait that confers a high tolerance to the pathogen Peronospora belbahrii, commonly known as Basil Downy Mildew. Plants of said basil are further valued as breeding lines enabling the development of superior basil plants having a tolerance to Basil Downy Mildew and other desirable traits, such as vigor and taste.

Another embodiment relates to a sweet basil plant, wherein said plant comprises at least one dominant allele and at least another recessive allele in the homozygous state, wherein when both said dominant and recessive alleles are present together in said states in the genome of said plant, are responsible for higher levels of tolerance to Peronospora belbahrii, and wherein a sample of representative seed of said sweet basil plants comprised of said genes responsible for higher levels of tolerance to Peronospora belbahrii is deposited under NCIMB No. 42726.

Another embodiment relates to tissue culture produced from protoplasts or cells from the basil plants disclosed in the subject application, wherein said cells or protoplasts are produced from a plant part selected from the group consisting of pollen, ovules, embryos, protoplasts, meristematic cells, callus, leaves, anthers, cotyledons, hypocotyl, pistils, roots, root tips, flowers, seeds, petiole, and stems.

Another embodiment relates to a method of vegetatively propagating a basil plant described in the present application, comprising the steps of: collecting tissue or cells capable of being propagated from said plant; cultivating said tissue or cells of to obtain proliferated shoots; and rooting said proliferated shoots to obtain rooted plantlets; or cultivating said tissue or cells to obtain proliferated shoots, or to obtain plantlets.

Another embodiment relates to a method of determining the genotype of the sweet basil plants described in the present application, wherein said method comprises obtaining a sample of nucleic acids from said plant and detecting in said nucleic acids a plurality of polymorphisms.

Another embodiment relates to a method for developing a basil plant in a basil plant breeding program, comprising applying plant breeding techniques comprising recurrent selection, backcrossing, pedigree breeding, marker enhanced selection, haploid/double haploid production, or transformation to the basil plants described in the present application, or its parts, wherein application of said techniques results in development of a new basil plant.

Another embodiment relates to a method of introducing a mutation into the genome of the sweet basil plants described in this application, said method comprising inducing a mutation to the plant, or plant part thereof, of the sweet basil plants described in the present application, wherein said induced mutation is selected from the group consisting of ionizing radiation, chemical mutagens, targeting induced local lesions in genomes, zinc finger nuclease mediated mutagenesis, meganucleases, and gene editing, and wherein the resulting plant comprises at least one genome mutation.

Another embodiment relates to a method of evaluating the response of an O. basilicum plant to Peronospora belbahrii, comprising the steps of: sowing O. basilicum seeds into one or more plug trays and placed in conditions to promote germination, wherein said seeds are comprised of one or more lines for testing, a susceptible control, and a resistant control; growing said plants in said plug trays to the stage where each plant has developed two true leaves, and inoculating said plants with Peronospora belbahrii inoculum solution, wherein the inoculum solution consist of fresh harvested leaves where sporulation has recently taken place that are placed in a container with water and agitated, and wherein the inoculum solution is adjusted to a concentration of 50,000 spores per milliliter of water using a hemocytometer, and wherein the inoculum solution is applied to the upper surface of the leaves using a hand sprayer; transferring the inoculated plants in the plug trays to a closed plastic chamber for 16 hours, wherein the conditions in the chamber are total darkness, a temperature range of 19 degrees Celsius to 21 degrees Celsius, a relative humidity range between 85% to 90% for the first 4 hours, and for hours 4 through 16, the relative humidity is greater than 90%; recording the relative humidity inside the plastic chamber; removing the plants in the plug trays from the plastic chamber and transferring said plants to a different location with full sunlight under natural day length and night length for 6 days; transferring the plants in the plug trays back into a plastic chamber for about 16 hours, wherein the conditions in the chamber are total darkness, a temperature range of 19 degrees Celsius to 21 degrees Celsius, a relative humidity range between 85% to 90% for the first 4 hours, and for hours 4 through 16, the relative humidity is greater than 90%; removing said plants and plug trays from the plastic chamber; observing and rating the degree of infection of Peronospora belbahrii disease development by evaluating the level of sporulation on the lower surface of each leaf of the plant.

In addition to the exemplary aspects and embodiments described above, further aspects and embodiments will become apparent by study of the following descriptions.

### BRIEF DESCRIPTION OF THE SEQUENCE LISTINGS

SEQ ID NO: 1 discloses a DNA sequence of O. basilicum having a SNP at position 61.

SEQ ID NO: 2 discloses a DNA sequence of O. basilicum having a SNP at position 61.

SEQ ID NO: 3 discloses a DNA sequence of O. basilicum having a SNP at position 61.

SEQ ID NO: 4 discloses a DNA sequence of O. basilicum having a SNP at position 61.

SEQ ID NO: 5 discloses a DNA sequence of O. basilicum having a SNP at position 61.

SEQ ID NO: 6 discloses a DNA sequence of O. basilicum having a SNP at position 61.

SEQ ID NO: 7 discloses a DNA sequence of O. basilicum having a SNP at position 61.

SEQ ID NO: 8 discloses a DNA sequence of O. basilicum having a SNP at position 61.

SEQ ID NO: 9 discloses a DNA sequence of O. basilicum having a SNP at position 61.

SEQ ID NO: 10 discloses a DNA sequence of O. basilicum having a SNP at position 61.

SEQ ID NO: 11 discloses a DNA sequence of O. basilicum having a SNP at position 61.

SEQ ID NO: 12 discloses a DNA sequence of O. basilicum having a SNP at position 61.

SEQ ID NO: 13 discloses a DNA sequence of O. basilicum having a SNP at position 61.

SEQ ID NO: 14 discloses a DNA sequence of O. basilicum having a SNP at position 61.

SEQ ID NO: 15 discloses a DNA sequence of O. basilicum having a SNP at position 61.

SEQ ID NO: 16 discloses a DNA sequence of O. basilicum having a SNP at position 61.

SEQ ID NO: 17 discloses a DNA sequence of O. basilicum having a SNP at position 61.

SEQ ID NO: 18 discloses a DNA sequence of O. basilicum having a SNP at position 61.

SEQ ID NO: 19 discloses a DNA sequence of O. basilicum having a SNP at position 61.

SEQ ID NO: 20 discloses a DNA sequence of O. basilicum having a SNP at position 61.

SEQ ID NO: 21 discloses a DNA sequence of O. basilicum having a SNP at position 61.

SEQ ID NO: 22 discloses a DNA sequence of O. basilicum having a SNP at position 61.

SEQ ID NO: 23 discloses a DNA sequence of O. basilicum having a SNP at position 61.

SEQ ID NO: 24 discloses a DNA sequence of O. basilicum having a SNP at position 61.

SEQ ID NO: 25 discloses a DNA sequence of O. basilicum having a SNP at position 61.

SEQ ID NO: 26 discloses a DNA sequence of O. basilicum having a SNP at position 61.

SEQ ID NO: 27 discloses a DNA sequence of O. basilicum having a SNP at position 61.

SEQ ID NO: 28 discloses a DNA sequence of O. basilicum having a SNP at position 61.

SEQ ID NO: 29 discloses a DNA sequence of O. basilicum having a SNP at position 61.

SEQ ID NO: 30 discloses a DNA sequence of O. basilicum having a SNP at position 61.

SEQ ID NO: 31 discloses a DNA sequence of O. basilicum having a SNP at position 61.

SEQ ID NO: 32 discloses a DNA sequence of O. basilicum having a SNP at position 61.

SEQ ID NO: 33 discloses a DNA sequence of O. basilicum having a SNP at position 61.

SEQ ID NO: 34 discloses a DNA sequence of O. basilicum having a SNP at position 61.

SEQ ID NO: 35 discloses a DNA sequence of O. basilicum having a SNP at position 61.

SEQ ID NO: 36 discloses a DNA sequence of O. basilicum having a SNP at position 61.

SEQ ID NO: 37 discloses a DNA sequence of O. basilicum having a SNP at position 61.

SEQ ID NO: 38 discloses a DNA sequence of O. basilicum having a SNP at position 61.

SEQ ID NO: 39 discloses a DNA sequence of O. basilicum having a SNP at position 61.

SEQ ID NO: 40 discloses a DNA sequence of O. basilicum having a SNP at position 61.

SEQ ID NO: 41 discloses a DNA sequence of O. basilicum having a SNP at position 61.

SEQ ID NO: 42 discloses a DNA sequence of O. basilicum having a SNP at position 61.

SEQ ID NO: 43 discloses a DNA sequence of O. basilicum having a SNP at position 61.

SEQ ID NO: 44 discloses a DNA sequence of O. basilicum having a SNP at position 61.

SEQ ID NO: 45 discloses a DNA sequence of O. basilicum having a SNP at position 61.

SEQ ID NO: 46 discloses a DNA sequence of O. basilicum having a SNP at position 61.

SEQ ID NO: 47 discloses a DNA sequence of O. basilicum having a SNP at position 61.

SEQ ID NO: 48 discloses a DNA sequence of O. basilicum having a SNP at position 61.

SEQ ID NO: 49 discloses a DNA sequence of O. basilicum having a SNP at position 61.

SEQ ID NO: 50 discloses a DNA sequence of O. basilicum having a SNP at position 61.

SEQ ID NO: 51 discloses a DNA sequence of O. basilicum having a SNP at position 61.

SEQ ID NO: 52 discloses a DNA sequence of O. basilicum having a SNP at position 61.

SEQ ID NO: 53 discloses a DNA sequence of O. basilicum having a SNP at position 61.

SEQ ID NO: 54 discloses a DNA sequence of O. basilicum having a SNP at position 61.

SEQ ID NO: 55 discloses a DNA sequence of O. basilicum having a SNP at position 61.

SEQ ID NO: 56 discloses a DNA sequence of O. basilicum having a SNP at position 61.

SEQ ID NO: 57 discloses a DNA sequence of O. basilicum having a SNP at position 61.

SEQ ID NO: 58 discloses a DNA sequence of O. basilicum having a SNP at position 61.

SEQ ID NO: 59 discloses a DNA sequence of O. basilicum having a SNP at position 61.

SEQ ID NO: 60 discloses a DNA sequence of O. basilicum having a SNP at position 61.

SEQ ID NO: 61 discloses a DNA sequence of O. basilicum having a SNP at position 61.

SEQ ID NO: 62 discloses a DNA sequence of O. basilicum having a SNP at position 61.

SEQ ID NO: 63 discloses a DNA sequence of O. basilicum having a SNP at position 61.

SEQ ID NO: 64 discloses a DNA sequence of O. basilicum having a SNP at position 61.

SEQ ID NO: 65 discloses a DNA sequence of O. basilicum having a SNP at position 61.

SEQ ID NO: 66 discloses a DNA sequence of O. basilicum having a SNP at position 61.

SEQ ID NO: 67 discloses a DNA sequence of O. basilicum having a SNP at position 61.

SEQ ID NO: 68 discloses a DNA sequence of O. basilicum having a SNP at position 61.

SEQ ID NO: 69 discloses a DNA sequence of O. basilicum having a SNP at position 61.

SEQ ID NO: 70 discloses a DNA sequence of O. basilicum having a SNP at position 61.

SEQ ID NO: 71 discloses a DNA sequence of O. basilicum having a SNP at position 61.

SEQ ID NO: 72 discloses a DNA sequence of O. basilicum having a SNP at position 61.

SEQ ID NO: 73 discloses a DNA sequence of O. basilicum having a SNP at position 61.

SEQ ID NO: 74 discloses a DNA sequence of O. basilicum having a SNP at position 61.

SEQ ID NO: 75 discloses a DNA sequence of O. basilicum having a SNP at position 61.

SEQ ID NO: 76 discloses a DNA sequence of O. basilicum having a SNP at position 61.

SEQ ID NO: 77 discloses a DNA sequence of O. basilicum having a SNP at position 61.

SEQ ID NO: 78 discloses a DNA sequence of O. basilicum having a SNP at position 61.

SEQ ID NO: 79 discloses a DNA sequence of O. basilicum having a SNP at position 61.

SEQ ID NO: 80 discloses a DNA sequence of O. basilicum having a SNP at position 61.

SEQ ID NO: 81 discloses a DNA sequence of O. basilicum having a SNP at position 61.

SEQ ID NO: 82 discloses a DNA sequence of O. basilicum having a SNP at position 61.

SEQ ID NO: 83 discloses a DNA sequence of O. basilicum having a SNP at position 61.

SEQ ID NO: 84 discloses a DNA sequence of O. basilicum having a SNP at position 61.

SEQ ID NO: 85 discloses a DNA sequence of O. basilicum having a SNP at position 61.

SEQ ID NO: 86 discloses a DNA sequence of O. basilicum having a SNP at position 61.

SEQ ID NO: 87 discloses a DNA sequence of O. basilicum having a SNP at position 61.

SEQ ID NO: 88 discloses a DNA sequence of O. basilicum having a SNP at position 61.

SEQ ID NO: 89 discloses a DNA sequence of O. basilicum having a SNP at position 61.

SEQ ID NO: 90 discloses a DNA sequence of O. basilicum having a SNP at position 61.

SEQ ID NO: 91 discloses a DNA sequence of O. basilicum having a SNP at position 61.

SEQ ID NO: 92 discloses a DNA sequence of O. basilicum having a SNP at position 61.

SEQ ID NO: 93 discloses a DNA sequence of O. basilicum having a SNP at position 61.

SEQ ID NO: 94 discloses a DNA sequence of O. basilicum having a SNP at position 61.

SEQ ID NO: 95 discloses a DNA sequence of O. basilicum having a SNP at position 61.

SEQ ID NO: 96 discloses a DNA sequence of O. basilicum having a SNP at position 61.

SEQ ID NO: 97 discloses a DNA sequence of O. basilicum having a SNP at position 61.

SEQ ID NO: 98 discloses a DNA sequence of O. basilicum having a SNP at position 61.

SEQ ID NO: 99 discloses a DNA sequence of O. basilicum having a SNP at position 61.

SEQ ID NO: 100 discloses a DNA sequence of O. basilicum having a SNP at position 61.

SEQ ID NO: 101 discloses a DNA sequence of O. basilicum having a SNP at position 61.

SEQ ID NO: 102 discloses a DNA sequence of O. basilicum having a SNP at position 61.

SEQ ID NO: 103 discloses a DNA sequence of O. basilicum having a SNP at position 61.

SEQ ID NO: 104 discloses a DNA sequence of O. basilicum having a SNP at position 61.

SEQ ID NO: 105 discloses a DNA sequence of O. basilicum having a SNP at position 61.

SEQ ID NO: 106 discloses a DNA sequence of O. basilicum having a SNP at position 61.

SEQ ID NO: 107 discloses a DNA sequence of O. basilicum having a SNP at position 61.

SEQ ID NO: 108 discloses a DNA sequence of O. basilicum having a SNP at position 61.

SEQ ID NO: 109 discloses a DNA sequence of O. basilicum having a SNP at position 61.

SEQ ID NO: 110 discloses a DNA sequence of O. basilicum having a SNP at position 61.

SEQ ID NO: 111 discloses a DNA sequence of O. basilicum having a SNP at position 61.

SEQ ID NO: 112 discloses a DNA sequence of O. basilicum having a SNP at position 61.

SEQ ID NO: 113 discloses a DNA sequence of O. basilicum having a SNP at position 61.

SEQ ID NO: 114 discloses a DNA sequence of O. basilicum having a SNP at position 61.

SEQ ID NO: 115 discloses a DNA sequence of Ocimum basilicum having a SNP at position 61.

SEQ ID NO: 116 discloses a DNA sequence of Ocimum basilicum having a SNP at position 61.

SEQ ID NO: 117 discloses a DNA sequence of Ocimum basilicum having a SNP at position 61.

SEQ ID NO: 118 discloses a DNA sequence of Ocimum basilicum having a SNP at position 61.

SEQ ID NO: 119 discloses a DNA sequence of Ocimum basilicum having a SNP at position 61.

SEQ ID NO: 120 discloses a DNA sequence of Ocimum basilicum having a SNP at position 61.

SEQ ID NO: 121 discloses a DNA sequence of Ocimum basilicum having a SNP at position 61.

SEQ ID NO: 122 discloses a DNA sequence of Ocimum basilicum having a SNP at position 61.

SEQ ID NO: 123 discloses a DNA sequence of Ocimum basilicum having a SNP at position 61.

SEQ ID NO: 124 discloses a DNA sequence of Ocimum basilicum having a SNP at position 61.

SEQ ID NO: 125 discloses a DNA sequence of Ocimum basilicum having a SNP at position 61.

SEQ ID NO: 126 discloses a DNA sequence of Ocimum basilicum having a SNP at position 61.

SEQ ID NO: 127 discloses a DNA sequence of Ocimum basilicum having a SNP at position 61.

SEQ ID NO: 128 discloses a DNA sequence of Ocimum basilicum having a SNP at 61.

SEQ ID NO: 129 discloses a DNA sequence of Ocimum basilicum having a SNP at position 61.

SEQ ID NO: 130 discloses a DNA sequence of Ocimum basilicum having a SNP at position 61.

SEQ ID NO: 131 discloses a DNA sequence of Ocimum basilicum having a SNP at position 61.

SEQ ID NO: 132 discloses a DNA sequence of Ocimum basilicum having a SNP at position 61.

SEQ ID NO: 133 discloses a DNA sequence of Ocimum basilicum having a SNP at position 61.

SEQ ID NO: 134 discloses a DNA sequence of Ocimum basilicum having a SNP at position 61.

SEQ ID NO: 135 discloses a DNA sequence of Ocimum basilicum having a SNP at position 61.

SEQ ID NO: 136 discloses a DNA sequence of Ocimum basilicum having a SNP at position 61.

SEQ ID NO: 137 discloses a DNA sequence of Ocimum basilicum having a SNP at position 61.

SEQ ID NO: 138 discloses a DNA sequence of Ocimum basilicum having a SNP at position 61.

SEQ ID NO: 139 discloses a DNA sequence of Ocimum basilicum having a SNP at position 61.

SEQ ID NO: 140 discloses a DNA sequence of Ocimum basilicum having a SNP at position 61.

SEQ ID NO: 141 discloses a DNA sequence of Ocimum basilicum having a SNP at position 61.

SEQ ID NO: 142 discloses a DNA sequence of Ocimum basilicum having a SNP at position 61.

SEQ ID NO: 143 discloses a DNA sequence of Ocimum basilicum having a SNP at 61.

SEQ ID NO: 144 discloses a DNA sequence of Ocimum basilicum having a SNP at position 61.

SEQ ID NO: 145 discloses a DNA sequence of Ocimum basilicum having a SNP at position 61.

SEQ ID NO: 146 discloses a DNA sequence of Ocimum basilicum having a SNP at position 61.

SEQ ID NO: 147 discloses a DNA sequence of Ocimum basilicum having a SNP at position 61.

SEQ ID NO: 148 discloses a DNA sequence of Ocimum basilicum having a SNP at position 61.

SEQ ID NO: 149 discloses a DNA sequence of Ocimum basilicum having a SNP at position 61.

SEQ ID NO: 150 discloses a DNA sequence of Ocimum basilicum having a SNP at position 61.

SEQ ID NO: 151 discloses a DNA sequence of Ocimum basilicum having a SNP at position 61.

SEQ ID NO: 152 discloses a DNA sequence of Ocimum basilicum having a SNP at position 61.

SEQ ID NO: 153 discloses a DNA sequence of Ocimum basilicum having a SNP at position 61.

SEQ ID NO: 154 discloses a DNA sequence of Ocimum basilicum having a SNP at position 61.

SEQ ID NO: 155 discloses a DNA sequence of Ocimum basilicum having a SNP at position 61.

SEQ ID NO: 156 discloses a DNA sequence of Ocimum basilicum having a SNP at position 61.

SEQ ID NO: 157 discloses a DNA sequence of Ocimum basilicum having a SNP at position 61.

SEQ ID NO: 158 discloses a DNA sequence of Ocimum basilicum having a SNP at position 61.

SEQ ID NO: 159 discloses a DNA sequence of Ocimum basilicum having a SNP at position 61.

SEQ ID NO: 160 discloses a DNA sequence of Ocimum basilicum having a SNP at position 61.

SEQ ID NO: 161 discloses a DNA sequence of Ocimum basilicum having a SNP at position 61.

SEQ ID NO: 162 discloses a DNA sequence of Ocimum basilicum having a SNP at position 61.

SEQ ID NO: 163 discloses a DNA sequence of Ocimum basilicum having a SNP at position 61.

SEQ ID NO: 164 discloses a DNA sequence of Ocimum basilicum having a SNP at position 61.

SEQ ID NO: 165 discloses a DNA sequence of Ocimum basilicum having a SNP at position 61.

SEQ ID NO: 166 discloses a DNA sequence of Ocimum basilicum having a SNP at position 61.

SEQ ID NO: 167 discloses a DNA sequence of Ocimum basilicum having a SNP at position 61.

SEQ ID NO: 168 discloses a DNA sequence of Ocimum basilicum having a SNP at position 61.

SEQ ID NO: 169 discloses a DNA sequence of Ocimum basilicum having a SNP at position 61.

SEQ ID NO: 170 discloses a DNA sequence of Ocimum basilicum having a SNP at position 61.

SEQ ID NO: 171 discloses a DNA sequence of Ocimum basilicum having a SNP at position 61.

SEQ ID NO: 172 discloses a DNA sequence of Ocimum basilicum having a SNP at position 61.

SEQ ID NO: 173 discloses a DNA sequence of Ocimum basilicum having a SNP at position 61.

SEQ ID NO: 174 discloses a DNA sequence of Ocimum basilicum having a SNP at position 61.

SEQ ID NO: 175 discloses a DNA sequence of Ocimum basilicum having a SNP at position 61.

SEQ ID NO: 176 discloses a DNA sequence of Ocimum basilicum having a SNP at position 61.

SEQ ID NO: 177 discloses a DNA sequence of Ocimum basilicum having a SNP at position 61.

SEQ ID NO: 178 discloses a DNA sequence of Ocimum basilicum having a SNP at position 61.

SEQ ID NO: 179 discloses a DNA sequence of Ocimum basilicum having a SNP at position 61.

SEQ ID NO: 180 discloses a DNA sequence of Ocimum basilicum having a SNP at position 61.

SEQ ID NO: 181 discloses a DNA sequence of Ocimum basilicum having a SNP at position 61.

SEQ ID NO: 182 discloses a DNA sequence of Ocimum basilicum having a SNP at position 61.

SEQ ID NO: 183 discloses a DNA sequence of Ocimum basilicum having a SNP at position 61.

SEQ ID NO: 184 discloses a DNA sequence of Ocimum basilicum having a SNP at position 61.

SEQ ID NO: 185 discloses a DNA sequence of Ocimum basilicum having a SNP at position 61.

SEQ ID NO: 186 discloses a DNA sequence of Ocimum basilicum having a SNP at position 61.

SEQ ID NO: 187 discloses a DNA sequence of Ocimum basilicum having a SNP at position 61.

SEQ ID NO: 188 discloses a DNA sequence of Ocimum basilicum having a SNP at position 61.

SEQ ID NO: 189 discloses a DNA sequence of Ocimum basilicum having a SNP at position 61.

SEQ ID NO: 190 discloses a DNA sequence of Ocimum basilicum having a SNP at position 61.

SEQ ID NO: 191 discloses a DNA sequence of Ocimum basilicum having a SNP at position 61.

SEQ ID NO: 192 discloses a DNA sequence of Ocimum basilicum having a SNP at position 61.

SEQ ID NO: 193 discloses a DNA sequence of Ocimum basilicum having a SNP at position 61.

SEQ ID NO: 194 discloses a DNA sequence of Ocimum basilicum having a SNP at position 61.

SEQ ID NO: 195 discloses a DNA sequence of Ocimum basilicum having a SNP at position 61.

SEQ ID NO: 196 discloses a DNA sequence of Ocimum basilicum having a SNP at position 61.

SEQ ID NO: 197 discloses a DNA sequence of Ocimum basilicum having a SNP at position 61.

SEQ ID NO: 198 discloses a DNA sequence of Ocimum basilicum having a SNP at position 61.

SEQ ID NO: 199 discloses a DNA sequence of Ocimum basilicum having a SNP at position 61.

SEQ ID NO: 200 discloses a DNA sequence of Ocimum basilicum having a SNP at position 61.

SEQ ID NO: 201 discloses a DNA sequence of Ocimum basilicum having a SNP at position 61.

SEQ ID NO: 202 discloses a DNA sequence of Ocimum basilicum having a SNP at position 61.

SEQ ID NO: 203 discloses a DNA sequence of Ocimum basilicum having a SNP at position 61.

SEQ ID NO: 204 discloses a DNA sequence of Ocimum basilicum having a SNP at position 61.

SEQ ID NO: 205 discloses a DNA sequence of Ocimum basilicum having a SNP at position 61.

SEQ ID NO: 206 discloses a DNA sequence of Ocimum basilicum having a SNP at position 61.

SEQ ID NO: 207 discloses a DNA sequence of Ocimum basilicum having a SNP at position 61.

SEQ ID NO: 208 discloses a DNA sequence of Ocimum basilicum having a SNP at position 61.

SEQ ID NO: 209 discloses a DNA sequence of Ocimum basilicum having a SNP at position 61.

SEQ ID NO: 210 discloses a DNA sequence of Ocimum basilicum having a SNP at position 61.

SEQ ID NO: 211 discloses a DNA sequence of Ocimum basilicum having a SNP at position 61.

SEQ ID NO: 212 discloses a DNA sequence of Ocimum basilicum having a SNP at position 61.

SEQ ID NO: 213 discloses a DNA sequence of Ocimum basilicum having a SNP at position 61.

SEQ ID NO: 214 discloses a DNA sequence of Ocimum basilicum having a SNP at position 61.

SEQ ID NO: 215 discloses a DNA sequence of Ocimum basilicum having a SNP at position 61.

SEQ ID NO: 216 discloses a DNA sequence of Ocimum basilicum having a SNP at position 61.

SEQ ID NO: 217 discloses a DNA sequence of Ocimum basilicum having a SNP at position 61.

SEQ ID NO: 218 discloses a DNA sequence of Ocimum basilicum having a SNP at position 61.

SEQ ID NO: 219 discloses a DNA sequence of Ocimum basilicum having a SNP at position 61.

SEQ ID NO: 220 discloses a DNA sequence of Ocimum basilicum having a SNP at position 61.

SEQ ID NO: 221 discloses a DNA sequence of Ocimum basilicum having a SNP at position 61.

SEQ ID NO: 222 discloses a DNA sequence of Ocimum basilicum having a SNP at position 61.

SEQ ID NO: 223 discloses a DNA sequence of Ocimum basilicum having a SNP at position 61.

SEQ ID NO: 224 discloses a DNA sequence of Ocimum basilicum having a SNP at position 61.

SEQ ID NO: 225 discloses a DNA sequence of Ocimum basilicum having a SNP at position 61.

SEQ ID NO: 226 discloses a DNA sequence of Ocimum basilicum having a SNP at position 61.

SEQ ID NO: 227 discloses a DNA sequence of Ocimum basilicum having a SNP at position 61.

SEQ ID NO: 228 discloses a DNA sequence of Ocimum basilicum having a SNP at position 61.

SEQ ID NO: 229 discloses a DNA sequence of Ocimum basilicum having a SNP at position 61.

SEQ ID NO: 230 discloses a DNA sequence of Ocimum basilicum having a SNP at position 61.

SEQ ID NO: 231 discloses a DNA sequence of Ocimum basilicum having a SNP at position 61.

SEQ ID NO: 232 discloses a DNA sequence of Ocimum basilicum having a SNP at position 61.

SEQ ID NO: 233 discloses a DNA sequence of Ocimum basilicum having a SNP at position 61.

SEQ ID NO: 234 discloses a DNA sequence of Ocimum basilicum having a SNP at position 61.

SEQ ID NO: 235 discloses a DNA sequence of Ocimum basilicum having a SNP at position 61.

SEQ ID NO: 236 discloses a DNA sequence of Ocimum basilicum having a SNP at position 61.

SEQ ID NO: 237 discloses a DNA sequence of Ocimum basilicum having a SNP at position 61.

SEQ ID NO: 238 discloses a DNA sequence of Ocimum basilicum having a SNP at position 61.

SEQ ID NO: 239 discloses a DNA sequence of Ocimum basilicum having a SNP at position 61.

SEQ ID NO: 240 discloses a DNA sequence of Ocimum basilicum having a SNP at position 61.

SEQ ID NO: 241 discloses a DNA sequence of Ocimum basilicum having a SNP at position 61.

SEQ ID NO: 242 discloses a DNA sequence of Ocimum basilicum having a SNP at position 61.

SEQ ID NO: 243 discloses a DNA sequence of Ocimum basilicum having a SNP at position 61.

SEQ ID NO: 244 discloses a DNA sequence of Ocimum basilicum having a SNP at position 61.

SEQ ID NO: 245 discloses a DNA sequence of Ocimum basilicum having a SNP at position 61.

SEQ ID NO: 246 discloses a DNA sequence of Ocimum basilicum having a SNP at position 61.

SEQ ID NO: 247 discloses a DNA sequence of Ocimum basilicum having a SNP at position 61.

SEQ ID NO: 248 discloses a DNA sequence of Ocimum basilicum having a SNP at position 61.

SEQ ID NO: 249 discloses a DNA sequence of Ocimum basilicum having a SNP at position 61.

SEQ ID NO: 250 discloses a DNA sequence of Ocimum basilicum having a SNP at position 61.

SEQ ID NO: 251 discloses a DNA sequence of Ocimum basilicum having a SNP at position 61.

SEQ ID NO: 252 discloses a DNA sequence of Ocimum basilicum having a SNP at position 61.

SEQ ID NO: 253 discloses a DNA sequence of Ocimum basilicum having a SNP at position 61.

SEQ ID NO: 254 discloses a DNA sequence of Ocimum basilicum having a SNP at position 61.

SEQ ID NO: 255 discloses a DNA sequence of Ocimum basilicum having a SNP at position 61.

SEQ ID NO: 256 discloses a DNA sequence of Ocimum basilicum having a SNP at position 61.

SEQ ID NO: 257 discloses a DNA sequence of Ocimum basilicum having a SNP at position 61.

SEQ ID NO: 258 discloses a DNA sequence of Ocimum basilicum having a SNP at position 61.

SEQ ID NO: 259 discloses a DNA sequence of Ocimum basilicum having a SNP at position 61.

SEQ ID NO: 260 discloses a DNA sequence of Ocimum basilicum having a SNP at position 61.

SEQ ID NO: 261 discloses a DNA sequence of Ocimum basilicum having a SNP at position 61.

SEQ ID NO: 262 discloses a DNA sequence of Ocimum basilicum having a SNP at position 61.

SEQ ID NO: 263 discloses a DNA sequence of Ocimum basilicum having a SNP at position 61.

SEQ ID NO: 264 discloses a DNA sequence of Ocimum basilicum having a SNP at position 61.

SEQ ID NO: 265 discloses a DNA sequence of Ocimum basilicum having a SNP at position 61.

SEQ ID NO: 266 discloses a DNA sequence of Ocimum basilicum having a SNP at position 61.

SEQ ID NO: 267 discloses a DNA sequence of Ocimum basilicum having a SNP at position 61.

SEQ ID NO: 268 discloses a DNA sequence of Ocimum basilicum having a SNP at position 61.

SEQ ID NO: 269 discloses a DNA sequence of Ocimum basilicum having a SNP at position 61.

SEQ ID NO: 270 discloses a DNA sequence of Ocimum basilicum having a SNP at position 61.

SEQ ID NO: 271 discloses a DNA sequence of Ocimum basilicum having a SNP at position 61.

SEQ ID NO: 272 discloses a DNA sequence of Ocimum basilicum having a SNP at position 61.

SEQ ID NO: 273 discloses a DNA sequence of Ocimum basilicum having a SNP at position 61.

SEQ ID NO: 274 discloses a DNA sequence of Ocimum basilicum having a SNP at position 61.

SEQ ID NO: 275 discloses a DNA sequence of Ocimum basilicum having a SNP at position 61.

SEQ ID NO: 276 discloses a DNA sequence of Ocimum basilicum having a SNP at position 61.

SEQ ID NO: 277 discloses a DNA sequence of Ocimum basilicum having a SNP at position 61.

SEQ ID NO: 278 discloses a DNA sequence of Ocimum basilicum having a SNP at position 61.

SEQ ID NO: 279 discloses a DNA sequence of Ocimum basilicum having a SNP at position 61.

SEQ ID NO: 280 discloses a DNA sequence of Ocimum basilicum having a SNP at position 61.

SEQ ID NO: 281 discloses a DNA sequence of Ocimum basilicum having a SNP at position 61.

SEQ ID NO: 282 discloses a DNA sequence of Ocimum basilicum having a SNP at position 61.

SEQ ID NO: 283 discloses a DNA sequence of Ocimum basilicum having a SNP at position 61.

SEQ ID NO: 284 discloses a DNA sequence of Ocimum basilicum having a SNP at position 61.

SEQ ID NO: 285 discloses a DNA sequence of Ocimum basilicum having a SNP at position 61.

SEQ ID NO: 286 discloses a DNA sequence of Ocimum basilicum having a SNP at position 61.

SEQ ID NO: 287 discloses a DNA sequence of Ocimum basilicum having a SNP at position 61.

SEQ ID NO: 288 discloses a DNA sequence of Ocimum basilicum having a SNP at position 61.

SEQ ID NO: 289 discloses a DNA sequence of Ocimum basilicum having a SNP at position 61.

SEQ ID NO: 290 discloses a DNA sequence of Ocimum basilicum having a SNP at position 61.

SEQ ID NO: 291 discloses a DNA sequence of Ocimum basilicum having a SNP at position 61.

SEQ ID NO: 292 discloses a DNA sequence of Ocimum basilicum having a SNP at position 61.

SEQ ID NO: 293 discloses a DNA sequence of Ocimum basilicum having a SNP at position 61.

SEQ ID NO: 294 discloses a DNA sequence of Ocimum basilicum having a SNP at position 61.

SEQ ID NO: 295 discloses a DNA sequence of Ocimum basilicum having a SNP at position 61.

SEQ ID NO: 296 discloses a DNA sequence of Ocimum basilicum having a SNP at position 61.

SEQ ID NO: 297 discloses a DNA sequence of Ocimum basilicum having a SNP at position 61.

SEQ ID NO: 298 discloses a DNA sequence of Ocimum basilicum having a SNP at position 61.

SEQ ID NO: 299 discloses a DNA sequence of Ocimum basilicum having a SNP at position 61.

SEQ ID NO: 300 discloses a DNA sequence of Ocimum basilicum having a SNP at position 61.

SEQ ID NO: 301 discloses a DNA sequence of Ocimum basilicum having a SNP at position 61.

SEQ ID NO: 302 discloses a DNA sequence of Ocimum basilicum having a SNP at position 61.

SEQ ID NO: 303 discloses a DNA sequence of Ocimum basilicum having a SNP at position 61.

SEQ ID NO: 304 discloses a DNA sequence of Ocimum basilicum having a SNP at position 61.

SEQ ID NO: 305 discloses a DNA sequence of Ocimum basilicum having a SNP at position 61.

SEQ ID NO: 306 discloses a DNA sequence of Ocimum basilicum having a SNP at position 61.

SEQ ID NO: 307 discloses a DNA sequence of Ocimum basilicum having a SNP at position 61.

SEQ ID NO: 308 discloses a DNA sequence of Ocimum basilicum having a SNP at position 61.

SEQ ID NO: 309 discloses a DNA sequence of Ocimum basilicum having a SNP at position 61.

SEQ ID NO: 310 discloses a DNA sequence of Ocimum basilicum having a SNP at position 61.

SEQ ID NO: 311 discloses a DNA sequence of Ocimum basilicum having a SNP at position 61.

SEQ ID NO: 312 discloses a DNA sequence of Ocimum basilicum having a SNP at position 61.

SEQ ID NO: 313 discloses a DNA sequence of Ocimum basilicum having a SNP at position 61.

SEQ ID NO: 314 discloses a DNA sequence of Ocimum basilicum having a SNP at position 61.

SEQ ID NO: 315 discloses a DNA sequence of Ocimum basilicum having a SNP at position 61.

SEQ ID NO: 316 discloses a DNA sequence of Ocimum basilicum having a SNP at position 61.

SEQ ID NO: 317 discloses a DNA sequence of Ocimum basilicum having a SNP at position 61.

SEQ ID NO: 318 discloses a DNA sequence of Ocimum basilicum having a SNP at position 61.

SEQ ID NO: 319 discloses a DNA sequence of Ocimum basilicum having a SNP at position 61.

SEQ ID NO: 320 discloses a DNA sequence of Ocimum basilicum having a SNP at position 61.

SEQ ID NO: 321 discloses a DNA sequence of Ocimum basilicum having a SNP at position 61.

SEQ ID NO: 322 discloses a DNA sequence of Ocimum basilicum having a SNP at position 61.

SEQ ID NO: 323 discloses a DNA sequence of Ocimum basilicum having a SNP at position 61.

SEQ ID NO: 324 discloses a DNA sequence of Ocimum basilicum having a SNP at position 61.

SEQ ID NO: 325 discloses a DNA sequence of Ocimum basilicum having a SNP at position 61.

SEQ ID NO: 326 discloses a DNA sequence of Ocimum basilicum having a SNP at position 61.

SEQ ID NO: 327 discloses a DNA sequence of Ocimum basilicum having a SNP at position 61.

SEQ ID NO: 328 discloses a DNA sequence of Ocimum basilicum having a SNP at position 61.

SEQ ID NO: 329 discloses a DNA sequence of Ocimum basilicum having a SNP at position 61.

SEQ ID NO: 330 discloses a DNA sequence of Ocimum basilicum having a SNP at position 61.

SEQ ID NO: 331 discloses a DNA sequence of Ocimum basilicum having a SNP at position 61.

SEQ ID NO: 332 discloses a DNA sequence of Ocimum basilicum having a SNP at position 61.

SEQ ID NO: 333 discloses a DNA sequence of Ocimum basilicum having a SNP at position 61.

SEQ ID NO: 334 discloses a DNA sequence of Ocimum basilicum having a SNP at position 61.

SEQ ID NO: 335 discloses a DNA sequence of Ocimum basilicum having a SNP at position 61.

SEQ ID NO: 336 discloses a DNA sequence of Ocimum basilicum having a SNP at position 61.

SEQ ID NO: 337 discloses a DNA sequence of Ocimum basilicum having a SNP at position 61.

SEQ ID NO: 338 discloses a DNA sequence of Ocimum basilicum having a SNP at position 61.

SEQ ID NO: 339 discloses a DNA sequence of Ocimum basilicum having a SNP at position 61.

SEQ ID NO: 340 discloses a DNA sequence of Ocimum basilicum having a SNP at position 61.

SEQ ID NO: 341 discloses a DNA sequence of Ocimum basilicum having a SNP at position 61.

SEQ ID NO: 342 discloses a DNA sequence of Ocimum basilicum having a SNP at position 61.

SEQ ID NO: 343 discloses a DNA sequence of Ocimum basilicum having a SNP at position 61.

SEQ ID NO: 344 discloses a DNA sequence of Ocimum basilicum having a SNP at position 61.

SEQ ID NO: 345 discloses a DNA sequence of Ocimum basilicum having a SNP at position 61.

SEQ ID NO: 346 discloses a DNA sequence of Ocimum basilicum having a SNP at position 61.

SEQ ID NO: 347 discloses a DNA sequence of Ocimum basilicum having a SNP at position 61.

SEQ ID NO: 348 discloses a DNA sequence of Ocimum basilicum having a SNP at position 61.

SEQ ID NO: 349 discloses a DNA sequence of Ocimum basilicum having a SNP at position 61.

SEQ ID NO: 350 discloses a DNA sequence of Ocimum basilicum having a SNP at position 61.

SEQ ID NO: 351 discloses a DNA sequence of Ocimum basilicum having a SNP at position 61.

SEQ ID NO: 352 discloses a DNA sequence of Ocimum basilicum having a SNP at position 61.

SEQ ID NO: 353 discloses a DNA sequence of Ocimum basilicum having a SNP at position 61.

SEQ ID NO: 354 discloses a DNA sequence of Ocimum basilicum having a SNP at position 61.

SEQ ID NO: 355 discloses a DNA sequence of Ocimum basilicum having a SNP at position 61.

SEQ ID NO: 356 discloses a DNA sequence of Ocimum basilicum having a SNP at position 61.

SEQ ID NO: 357 discloses a DNA sequence of Ocimum basilicum having a SNP at position 61.

SEQ ID NO: 358 discloses a DNA sequence of Ocimum basilicum having a SNP at position 61.

SEQ ID NO: 359 discloses a DNA sequence of Ocimum basilicum having a SNP at position 61.

SEQ ID NO: 360 discloses a DNA sequence of Ocimum basilicum having a SNP at position 61.

SEQ ID NO: 361 discloses a DNA sequence of Ocimum basilicum having a SNP at position 61.

SEQ ID NO: 362 discloses a DNA sequence of Ocimum basilicum having a SNP at position 61.

SEQ ID NO: 363 discloses a DNA sequence of Ocimum basilicum having a SNP at position 61.

SEQ ID NO: 364 discloses a DNA sequence of Ocimum basilicum having a SNP at position 61.

SEQ ID NO: 365 discloses a DNA sequence of Ocimum basilicum having a SNP at position 61.

SEQ ID NO: 366 discloses a DNA sequence of Ocimum basilicum having a SNP at position 61.

SEQ ID NO: 367 discloses a DNA sequence of Ocimum basilicum having a SNP at position 61.

SEQ ID NO: 368 discloses a DNA sequence of Ocimum basilicum having a SNP at position 61.

SEQ ID NO: 369 discloses a DNA sequence of Ocimum basilicum having a SNP at position 61.

SEQ ID NO: 370 discloses a DNA sequence of Ocimum basilicum having a SNP at position 61.

SEQ ID NO: 371 discloses a DNA sequence of Ocimum basilicum having a SNP at position 61.

SEQ ID NO: 372 discloses a DNA sequence of Ocimum basilicum having a SNP at position 61.

SEQ ID NO: 373 discloses a DNA sequence of Ocimum basilicum having a SNP at position 61.

SEQ ID NO: 374 discloses a DNA sequence of Ocimum basilicum having a SNP at position 61.

SEQ ID NO: 375 discloses a DNA sequence of Ocimum basilicum having a SNP at position 61.

SEQ ID NO: 376 discloses a DNA sequence of Ocimum basilicum having a SNP at position 61.

SEQ ID NO: 377 discloses a DNA sequence of Ocimum basilicum having a SNP at position 61.

SEQ ID NO: 378 discloses a DNA sequence of Ocimum basilicum having a SNP at position 61.

SEQ ID NO: 379 discloses a DNA sequence of Ocimum basilicum having a SNP at position 61.

SEQ ID NO: 380 discloses a DNA sequence of Ocimum basilicum having a SNP at position 61.

SEQ ID NO: 381 discloses a DNA sequence of Ocimum basilicum having a SNP at position 61.

SEQ ID NO: 382 discloses a DNA sequence of Ocimum basilicum having a SNP at position 61.

SEQ ID NO: 383 discloses a DNA sequence of Ocimum basilicum having a SNP at position 61.

SEQ ID NO: 384 discloses a DNA sequence of Ocimum basilicum having a SNP at position 61.

SEQ ID NO: 385 discloses a DNA sequence of Ocimum basilicum having a SNP at position 61.

SEQ ID NO: 386 discloses a DNA sequence of Ocimum basilicum having a SNP at position 61.

SEQ ID NO: 387 discloses a DNA sequence of Ocimum basilicum having a SNP at position 61.

SEQ ID NO: 388 discloses a DNA sequence of Ocimum basilicum having a SNP at position 61.

SEQ ID NO: 389 discloses a DNA sequence of Ocimum basilicum having a SNP at position 61.

SEQ ID NO: 390 discloses a DNA sequence of Ocimum basilicum having a SNP at position 61.

SEQ ID NO: 391 discloses a DNA sequence of Ocimum basilicum having a SNP at position 61.

SEQ ID NO: 392 discloses a DNA sequence of Ocimum basilicum having a SNP at position 61.

SEQ ID NO: 393 discloses a DNA sequence of Ocimum basilicum having a SNP at position 61.

SEQ ID NO: 394 discloses a DNA sequence of Ocimum basilicum having a SNP at position 61.

SEQ ID NO: 395 discloses a DNA sequence of Ocimum basilicum having a SNP at position 61.

SEQ ID NO: 396 discloses a DNA sequence of Ocimum basilicum having a SNP at position 61.

SEQ ID NO: 397 discloses a DNA sequence of Ocimum basilicum having a SNP at position 61.

SEQ ID NO: 398 discloses a DNA sequence of Ocimum basilicum having a SNP at position 61.

SEQ ID NO: 399 discloses a DNA sequence of Ocimum basilicum having a SNP at position 61.

SEQ ID NO: 400 discloses a DNA sequence of Ocimum basilicum having a SNP at position 61.

SEQ ID NO: 401 discloses a DNA sequence of Ocimum basilicum having a SNP at position 61.

SEQ ID NO: 402 discloses a DNA sequence of Ocimum basilicum having a SNP at position 61.

SEQ ID NO: 403 discloses a DNA sequence of Ocimum basilicum having a SNP at position 61.

SEQ ID NO: 404 discloses a DNA sequence of Ocimum basilicum having a SNP at position 61.

SEQ ID NO: 405 discloses a DNA sequence of Ocimum basilicum having a SNP at position 61.

SEQ ID NO: 406 discloses a DNA sequence of Ocimum basilicum having a SNP at position 61.

SEQ ID NO: 407 discloses a DNA sequence of Ocimum basilicum having a SNP at position 61.

SEQ ID NO: 408 discloses a DNA sequence of Ocimum basilicum having a SNP at position 61.

SEQ ID NO: 409 discloses a DNA sequence of Ocimum basilicum having a SNP at position 61.

SEQ ID NO: 410 discloses a DNA sequence of Ocimum basilicum having a SNP at position 61.

SEQ ID NO: 411 discloses a DNA sequence of Ocimum basilicum having a SNP at position 61.

SEQ ID NO: 412 discloses a DNA sequence of Ocimum basilicum having a SNP at position 61.

SEQ ID NO: 413 discloses a DNA sequence of Ocimum basilicum having a SNP at position 61.

SEQ ID NO: 414 discloses a DNA sequence of Ocimum basilicum having a SNP at position 61.

SEQ ID NO: 415 discloses a DNA sequence of Ocimum basilicum having a SNP at position 61.

SEQ ID NO: 416 discloses a DNA sequence of Ocimum basilicum having a SNP at position 61.

SEQ ID NO: 417 discloses a DNA sequence of Ocimum basilicum having a SNP at position 61.

SEQ ID NO: 418 discloses a DNA sequence of Ocimum basilicum having a SNP at position 61.

SEQ ID NO: 419 discloses a DNA sequence of Ocimum basilicum having a SNP at position 61.

SEQ ID NO: 420 discloses a DNA sequence of Ocimum basilicum having a SNP at position 61.

SEQ ID NO: 421 discloses a DNA sequence of Ocimum basilicum having a SNP at position 61.

SEQ ID NO: 422 discloses a DNA sequence of Ocimum basilicum having a SNP at position 61.

SEQ ID NO: 423 discloses a DNA sequence of Ocimum basilicum having a SNP at position 61.

SEQ ID NO: 424 discloses a DNA sequence of Ocimum basilicum having a SNP at position 61.

SEQ ID NO: 425 discloses a DNA sequence of Ocimum basilicum having a SNP at position 61.

SEQ ID NO: 426 discloses a DNA sequence of Ocimum basilicum having a SNP at position 61.

SEQ ID NO: 427 discloses a DNA sequence of Ocimum basilicum having a SNP at position 61.

SEQ ID NO: 428 discloses a DNA sequence of Ocimum basilicum having a SNP at position 61.

SEQ ID NO: 429 discloses a DNA sequence of Ocimum basilicum having a SNP at position 61.

SEQ ID NO: 430 discloses a DNA sequence of Ocimum basilicum having a SNP at position 55.

SEQ ID NO: 431 discloses a DNA sequence of Ocimum basilicum having a SNP at position 61.

SEQ ID NO: 432 discloses a DNA sequence of Ocimum basilicum having a SNP at position 61.

SEQ ID NO: 433 discloses a DNA sequence of Ocimum basilicum having a SNP at position 61.

SEQ ID NO: 434 discloses a DNA sequence of Ocimum basilicum having a SNP at position 61

SEQ ID NO: 435 discloses a DNA sequence of Ocimum basilicum having a SNP at position 61.

SEQ ID NO: 436 discloses a DNA sequence of Ocimum basilicum having a SNP at position 61.

SEQ ID NO: 437 discloses a DNA sequence of Ocimum basilicum having a SNP at position 61.

SEQ ID NO: 438 discloses a DNA sequence of Ocimum basilicum having a SNP at position 61.

SEQ ID NO: 439 discloses a DNA sequence of Ocimum basilicum having a SNP at position 61.

SEQ ID NO: 440 discloses a DNA sequence of Ocimum basilicum having a SNP at position 61.

SEQ ID NO: 441 discloses a DNA sequence of Ocimum basilicum having a SNP at position 61.

SEQ ID NO: 442 discloses a DNA sequence of Ocimum basilicum having a SNP at position 61.

SEQ ID NO: 443 discloses a DNA sequence of Ocimum basilicum having a SNP at position 61.

SEQ ID NO: 444 discloses a DNA sequence of Ocimum basilicum having a SNP at position 61.

SEQ ID NO: 445 discloses a DNA sequence of Ocimum basilicum having a SNP at position 61.

SEQ ID NO: 446 discloses a DNA sequence of Ocimum basilicum having a SNP at position 61.

SEQ ID NO: 447 discloses a DNA sequence of Ocimum basilicum having a SNP at position 61.

SEQ ID NO: 448 discloses a DNA sequence of Ocimum basilicum having a SNP at position 61.

SEQ ID NO: 449 discloses a DNA sequence of Ocimum basilicum having a SNP at position 61.

SEQ ID NO: 450 discloses a DNA sequence of Ocimum basilicum having a SNP at position 61.

SEQ ID NO: 451 discloses a DNA sequence of Ocimum basilicum having a SNP at position 61.

SEQ ID NO: 452 discloses a DNA sequence of Ocimum basilicum having a SNP at position 61.

SEQ ID NO: 453 discloses a DNA sequence of Ocimum basilicum having a SNP at position 61.

SEQ ID NO: 454 discloses a DNA sequence of Ocimum basilicum having a SNP at position 61.

SEQ ID NO: 455 discloses a DNA sequence of Ocimum basilicum having a SNP at position 61.

SEQ ID NO: 456 discloses a DNA sequence of Ocimum basilicum having a SNP at position 61.

SEQ ID NO: 457 discloses a DNA sequence of Ocimum basilicum having a SNP at position 61.

SEQ ID NO: 458 discloses a DNA sequence of Ocimum basilicum having a SNP at position 61.

SEQ ID NO: 459 discloses a DNA sequence of Ocimum basilicum having a SNP at position 61.

SEQ ID NO: 460 discloses a DNA sequence of Ocimum basilicum having a SNP at position 61.

SEQ ID NO: 461 discloses a DNA sequence of Ocimum basilicum having a SNP at position 61.

SEQ ID NO: 462 discloses a DNA sequence of Ocimum basilicum having a SNP at position 61.

SEQ ID NO: 463 discloses a DNA sequence of Ocimum basilicum having a SNP at position 61.

SEQ ID NO: 464 discloses a DNA sequence of Ocimum basilicum having a SNP at position 61.

SEQ ID NO: 465 discloses a DNA sequence of Ocimum basilicum having a SNP at position 61.

SEQ ID NO: 466 discloses a DNA sequence of Ocimum basilicum having a SNP at position 61.

SEQ ID NO: 467 discloses a DNA sequence of Ocimum basilicum having a SNP at position 61.

SEQ ID NO: 468 discloses a DNA sequence of Ocimum basilicum having a SNP at position 61.

SEQ ID NO: 469 discloses a DNA sequence of Ocimum basilicum having a SNP at position 61.

SEQ ID NO: 470 discloses a DNA sequence of Ocimum basilicum having a SNP at position 61.

SEQ ID NO: 471 discloses a DNA sequence of Ocimum basilicum having a SNP at position 61.

SEQ ID NO: 472 discloses a DNA sequence of Ocimum basilicum having a SNP at position 61.

SEQ ID NO: 473 discloses a DNA sequence of Ocimum basilicum having a SNP at position 61.

SEQ ID NO: 474 discloses a DNA sequence of Ocimum basilicum having a SNP at position 61.

SEQ ID NO: 475 discloses a DNA sequence of Ocimum basilicum having a SNP at position 61.

SEQ ID NO: 476 discloses a DNA sequence of Ocimum basilicum having a SNP at position 61.

SEQ ID NO: 477 discloses a DNA sequence of Ocimum basilicum having a SNP at position 61.

SEQ ID NO: 478 discloses a DNA sequence of Ocimum basilicum having a SNP at position 61.

SEQ ID NO: 479 discloses a DNA sequence of Ocimum basilicum having a SNP at position 61.

SEQ ID NO: 480 discloses a DNA sequence of Ocimum basilicum having a SNP at position 61.

SEQ ID NO: 481 discloses a DNA sequence of Ocimum basilicum having a SNP at position 61.

SEQ ID NO: 482 discloses a DNA sequence of Ocimum basilicum having a SNP at position 61.

SEQ ID NO: 483 discloses a DNA sequence of Ocimum basilicum having a SNP at position 61.

SEQ ID NO: 484 discloses a DNA sequence of Ocimum basilicum having a SNP at position 61.

SEQ ID NO: 485 discloses a DNA sequence of Ocimum basilicum having a SNP at position 61.

SEQ ID NO: 486 discloses a DNA sequence of Ocimum basilicum having a SNP at position 61.

SEQ ID NO: 487 discloses a DNA sequence of Ocimum basilicum having a SNP at position 61.

SEQ ID NO: 488 discloses a DNA sequence of Ocimum basilicum having a SNP at position 61.

SEQ ID NO: 489 discloses a DNA sequence of Ocimum basilicum having a SNP at position 61.

SEQ ID NO: 490 discloses a DNA sequence of Ocimum basilicum having a SNP at position 61.

SEQ ID NO: 491 discloses a DNA sequence of Ocimum basilicum having a SNP at position 61.

SEQ ID NO: 492 discloses a DNA sequence of Ocimum basilicum having a SNP at position 61.

SEQ ID NO: 493 discloses a DNA sequence of Ocimum basilicum having a SNP at position 61.

SEQ ID NO: 494 discloses a DNA sequence of Ocimum basilicum having a SNP at position 61.

SEQ ID NO: 495 discloses a DNA sequence of Ocimum basilicum having a SNP at position 61.

SEQ ID NO: 496 discloses a DNA sequence of Ocimum basilicum having a SNP at position 61.

SEQ ID NO: 497 discloses a DNA sequence of Ocimum basilicum having a SNP at position 61.

SEQ ID NO: 498 discloses a DNA sequence of Ocimum basilicum having a SNP at position 61.

SEQ ID NO: 499 discloses a DNA sequence of Ocimum basilicum having a SNP at position 61.

SEQ ID NO: 500 discloses a DNA sequence of Ocimum basilicum having a SNP at position 61.

SEQ ID NO: 501 discloses a DNA sequence of O. basilicum having a SNP at position 61.

SEQ ID NO: 502 discloses a DNA sequence of O. basilicum having a SNP at position 61.

SEQ ID NO: 503 discloses a DNA sequence of O. basilicum having a SNP at position 61.

SEQ ID NO: 504 discloses a DNA sequence of O. basilicum having a SNP at position 61.

SEQ ID NO: 505 discloses a DNA sequence of O. basilicum having a SNP at position 61.

SEQ ID NO: 506 discloses a DNA sequence of O. basilicum having a SNP at position 61.

SEQ ID NO: 507 discloses a DNA sequence of O. basilicum having a SNP at position 61.

SEQ ID NO: 508 discloses a DNA sequence of O. basilicum having a SNP at position 61.

SEQ ID NO: 509 discloses a DNA sequence of O. basilicum having a SNP at position 61.

SEQ ID NO: 510 discloses a DNA sequence of O. basilicum having a SNP at position 61.

SEQ ID NO: 511 discloses a DNA sequence of O. basilicum having a SNP at position 61.

SEQ ID NO: 512 discloses a DNA sequence of O. basilicum having a SNP at position 61.

SEQ ID NO: 513 discloses a DNA sequence of O. basilicum having a SNP at position 61.

SEQ ID NO: 514 discloses a DNA sequence of O. basilicum having a SNP at position 61.

SEQ ID NO: 515 discloses a DNA sequence of O. basilicum having a SNP at position 61.

SEQ ID NO: 516 discloses a DNA sequence of O. basilicum having a SNP at position 61.

SEQ ID NO: 517 discloses a DNA sequence of O. basilicum having a SNP at position 61.

SEQ ID NO: 518 discloses a DNA sequence of O. basilicum having a SNP at position 61.

SEQ ID NO: 519 discloses a DNA sequence of O. basilicum having a SNP at position 61.

SEQ ID NO: 520 discloses a DNA sequence of O. basilicum having a SNP at position 61.

SEQ ID NO: 521 discloses a DNA sequence of O. basilicum having a SNP at position 61.

SEQ ID NO: 522 discloses a DNA sequence of O. basilicum having a SNP at position 61.

SEQ ID NO: 523 discloses a DNA sequence of O. basilicum having a SNP at position 61.

SEQ ID NO: 524 discloses a DNA sequence of O. basilicum having a SNP at position 61.

SEQ ID NO: 525 discloses a DNA sequence of O. basilicum having a SNP at position 61.

SEQ ID NO: 526 discloses a DNA sequence of O. basilicum having a SNP at position 61.

SEQ ID NO: 527 discloses a DNA sequence of O. basilicum having a SNP at position 61.

SEQ ID NO: 528 discloses a DNA sequence of O. basilicum having a SNP at position 61.

SEQ ID NO: 529 discloses a DNA sequence of O. basilicum having a SNP at position 61.

SEQ ID NO: 530 discloses a DNA sequence of O. basilicum having a SNP at position 61.

SEQ ID NO: 531 discloses a DNA sequence of O. basilicum having a SNP at position 61.

SEQ ID NO: 532 discloses a DNA sequence of O. basilicum having a SNP at position 61.

SEQ ID NO: 533 discloses a DNA sequence of O. basilicum having a SNP at position 61.

SEQ ID NO: 534 discloses a DNA sequence of O. basilicum having a SNP at position 61.

SEQ ID NO: 535 discloses a DNA sequence of O. basilicum having a SNP at position 61.

SEQ ID NO: 536 discloses a DNA sequence of O. basilicum having a SNP at position 61.

SEQ ID NO: 537 discloses a DNA sequence of O. basilicum having a SNP at position 61.

SEQ ID NO: 538 discloses a DNA sequence of O. basilicum having a SNP at position 61.

SEQ ID NO: 539 discloses a DNA sequence of O. basilicum having a SNP at position 61.

SEQ ID NO: 540 discloses a DNA sequence of O. basilicum having a SNP at position 61.

SEQ ID NO: 541 discloses a DNA sequence of O. basilicum having a SNP at position 61.

SEQ ID NO: 542 discloses a DNA sequence of O. basilicum having a SNP at position 61.

SEQ ID NO: 543 discloses a DNA sequence of O. basilicum having a SNP at position 61.

SEQ ID NO: 544 discloses a DNA sequence of O. basilicum having a SNP at position 61.

SEQ ID NO: 545 discloses a DNA sequence of O. basilicum having a SNP at position 61.

SEQ ID NO: 546 discloses a DNA sequence of O. basilicum having a SNP at position 61.

SEQ ID NO: 547 discloses a DNA sequence of O. basilicum having a SNP at position 61.

SEQ ID NO: 548 discloses a DNA sequence of O. basilicum having a SNP at position 61.

SEQ ID NO: 549 discloses a DNA sequence of O. basilicum having a SNP at position 61.

SEQ ID NO: 550 discloses a DNA sequence of O. basilicum having a SNP at position 61.

SEQ ID NO: 551 discloses a DNA sequence of O. basilicum having a SNP at position 61.

SEQ ID NO: 552 discloses a DNA sequence of O. basilicum having a SNP at position 61.

SEQ ID NO: 553 discloses a DNA sequence of O. basilicum having a SNP at position 61.

SEQ ID NO: 554 discloses a DNA sequence of O. basilicum having a SNP at position 61.

SEQ ID NO: 555 discloses a DNA sequence of O. basilicum having a SNP at position 61.

SEQ ID NO: 556 discloses a DNA sequence of O. basilicum having a SNP at position 61.

SEQ ID NO: 557 discloses a DNA sequence of O. basilicum having a SNP at position 61.

SEQ ID NO: 558 discloses a DNA sequence of O. basilicum having a SNP at position 61.

SEQ ID NO: 559 discloses a DNA sequence of O. basilicum having a SNP at position 61.

SEQ ID NO: 560 discloses a DNA sequence of O. basilicum having a SNP at position 61.

SEQ ID NO: 561 discloses a DNA sequence of O. basilicum having a SNP at position 61.

SEQ ID NO: 562 discloses a DNA sequence of O. basilicum having a SNP at position 61.

SEQ ID NO: 563 discloses a DNA sequence of O. basilicum having a SNP at position 61.

SEQ ID NO: 564 discloses a DNA sequence of O. basilicum having a SNP at position 61.

SEQ ID NO: 565 discloses a DNA sequence of O. basilicum having a SNP at position 61.

SEQ ID NO: 566 discloses a DNA sequence of O. basilicum having a SNP at position 61.

SEQ ID NO: 567 discloses a DNA sequence of O. basilicum having a SNP at position 61.

SEQ ID NO: 568 discloses a DNA sequence of O. basilicum having a SNP at position 61.

SEQ ID NO: 569 discloses a DNA sequence of O. basilicum having a SNP at position 61.

SEQ ID NO: 570 discloses a DNA sequence of O. basilicum having a SNP at position 61.

SEQ ID NO: 571 discloses a DNA sequence of O. basilicum having a SNP at position 61.

SEQ ID NO: 572 discloses a DNA sequence of O. basilicum having a SNP at position 61.

SEQ ID NO: 573 discloses a DNA sequence of O. basilicum having a SNP at position 61.

SEQ ID NO: 574 discloses a DNA sequence of O. basilicum having a SNP at position 61.

SEQ ID NO: 575 discloses a DNA sequence of O. basilicum having a SNP at position 61.

SEQ ID NO: 576 discloses a DNA sequence of O. basilicum having a SNP at position 61.

SEQ ID NO: 577 discloses a DNA sequence of O. basilicum having a SNP at position 61.

SEQ ID NO: 578 discloses a DNA sequence of O. basilicum having a SNP at position 61.

SEQ ID NO: 579 discloses a DNA sequence of O. basilicum having a SNP at position 61.

SEQ ID NO: 580 discloses a DNA sequence of O. basilicum having a SNP at position 61.

SEQ ID NO: 581 discloses a DNA sequence of O. basilicum having a SNP at position 61.

SEQ ID NO: 582 discloses a DNA sequence of O. basilicum having a SNP at position 61.

SEQ ID NO: 583 discloses a DNA sequence of O. basilicum having a SNP at position 61.

SEQ ID NO: 584 discloses a DNA sequence of O. basilicum having a SNP at position 61.

SEQ ID NO: 585 discloses a DNA sequence of O. basilicum having a SNP at position 61.

SEQ ID NO: 586 discloses a DNA sequence of O. basilicum having a SNP at position 61.

SEQ ID NO: 587 discloses a DNA sequence of O. basilicum having a SNP at position 61.

SEQ ID NO: 588 discloses a DNA sequence of O. basilicum having a SNP at position 61.

SEQ ID NO: 589 discloses a DNA sequence of O. basilicum having a SNP at position 61.

SEQ ID NO: 590 discloses a DNA sequence of O. basilicum having a SNP at position 61.

SEQ ID NO: 591 discloses a DNA sequence of O. basilicum having a SNP at position 61.

SEQ ID NO: 592 discloses a DNA sequence of O. basilicum having a SNP at position 61.

SEQ ID NO: 593 discloses a DNA sequence of O. basilicum having a SNP at position 61.

SEQ ID NO: 594 discloses a DNA sequence of O. basilicum having a SNP at position 61.

SEQ ID NO: 595 discloses a DNA sequence of O. basilicum having a SNP at position 61.

SEQ ID NO: 596 discloses a DNA sequence of O. basilicum having a SNP at position 61.

SEQ ID NO: 597 discloses a DNA sequence of O. basilicum having a SNP at position 61.

SEQ ID NO: 598 discloses a DNA sequence of O. basilicum having a SNP at position 61.

SEQ ID NO: 599 discloses a DNA sequence of O. basilicum having a SNP at position 61.

SEQ ID NO: 600 discloses a DNA sequence of O. basilicum having a SNP at position 61.

SEQ ID NO: 601 discloses a DNA sequence of O. basilicum having a SNP at position 61.

SEQ ID NO: 602 discloses a DNA sequence of O. basilicum having a SNP at position 61.

SEQ ID NO: 603 discloses a DNA sequence of O. basilicum having a SNP at position 61.

SEQ ID NO: 604 discloses a DNA sequence of O. basilicum having a SNP at position 61.

SEQ ID NO: 605 discloses a DNA sequence of O. basilicum having a SNP at position 61.

SEQ ID NO: 606 discloses a DNA sequence of O. basilicum having a SNP at position 61.

SEQ ID NO: 607 discloses a DNA sequence of O. basilicum having a SNP at position 61.

SEQ ID NO: 608 discloses a DNA sequence of O. basilicum having a SNP at position 61.

SEQ ID NO: 609 discloses a DNA sequence of O. basilicum having a SNP at position 61.

SEQ ID NO: 610 discloses a DNA sequence of O. basilicum having a SNP at position 61.

SEQ ID NO: 611 discloses a DNA sequence of O. basilicum having a SNP at position 61.

SEQ ID NO: 612 discloses a DNA sequence of O. basilicum having a SNP at position 61.

SEQ ID NO: 613 discloses a DNA sequence of O. basilicum having a SNP at position 61.

SEQ ID NO: 614 discloses a DNA sequence of O. basilicum having a SNP at position 61.

SEQ ID NO: 615 discloses a DNA sequence of O. basilicum having a SNP at position 61.

SEQ ID NO: 616 discloses a DNA sequence of O. basilicum having a SNP at position 61.

SEQ ID NO: 617 discloses a DNA sequence of O. basilicum having a SNP at position 61.

SEQ ID NO: 618 discloses a DNA sequence of O. basilicum having a SNP at position 61.

SEQ ID NO: 619 discloses a DNA sequence of O. basilicum having a SNP at position 61.

SEQ ID NO: 620 discloses a DNA sequence of O. basilicum having a SNP at position 61.

SEQ ID NO: 621 discloses a DNA sequence of O. basilicum having a SNP at position 61.

SEQ ID NO: 622 discloses a DNA sequence of O. basilicum having a SNP at position 61.

SEQ ID NO: 623 discloses a DNA sequence of O. basilicum having a SNP at position 61.

SEQ ID NO: 624 discloses a DNA sequence of O. basilicum having a SNP at position 61.

SEQ ID NO: 625 discloses a DNA sequence of O. basilicum having a SNP at position 61.

SEQ ID NO: 626 discloses a DNA sequence of O. basilicum having a SNP at position 61.

SEQ ID NO: 627 discloses a DNA sequence of O. basilicum having a SNP at position 61.

SEQ ID NO: 628 discloses a DNA sequence of O. basilicum having a SNP at position 61.

SEQ ID NO: 629 discloses a DNA sequence of O. basilicum having a SNP at position 61.

SEQ ID NO: 630 discloses a DNA sequence of O. basilicum having a SNP at position 61.

### BRIEF DESCRIPTION OF THE FIGURES

FIG. 1 is a photo showing the female/seed B1020 basil parent as grown in a research field in San Jose del Cabo, Mexico.

FIG. 2 is a photo showing the female B1020 basil plants having Fusarium wilt symptoms.

FIG. 3 is a photo showing the flowering structure of the female B1020 basil parent.

FIG. 4 is a close-up under magnification showing the reaction of the female B1020 basil plants to high pressure of Basil Downy Mildew.

FIG. 5 is a photo of the B1020 OP1.

FIG. 6 is a photo of B1020 OP1 flowering inside a shade house.

FIGS. 7 A-D are photos of the selection BHT-1 showing the overall plant habit, the length of the mature leaf, the top view of the plant, and the internodes and stem.

FIGS. 8 A-D are photos of the selection BHT-2 showing the overall plant habit, the length of the mature leaf, the top view of the plant, and the internodes and stem.

FIG. 9 is a photo showing a comparison between the leaf appearance from left to right, the commercial variety ‘Nufar’, BHT-1, and BHT-2.

### DEFINITIONS

In the description and tables herein, a number of terms are used. In order to provide a clear and consistent understanding of the specification and claims, including the scope to be given such terms, the following definitions are provided:

Allele. Allele is any of one or more alternative forms for a gene.

Basil Downy Mildew. As used herein, “Basil Downy Mildew” refers to a disease caused by the fungal pathogen Peronospora belbahrii. Infected plants initially show chlorotic leaves, irregular necrotic black areas on the leaves, and fungal spores are typically found on leaves which previously remained wet greater than four hours providing for leaf infection.

Gene. As used herein, “gene” refers to a segment of nucleic acid.

Locus. A locus is the position or location of a gene on a chromosome.

Marker: As used herein, a “marker” is an indicator for the presence of at least one polymorphism, thus a marker can be the nucleotide sequence itself, or a probe, for example.

Plant. As used herein, the term “plant” includes plant cells, plant protoplasts, plant cell tissue cultures from which basil plants can be regenerated, plant calli, plant clumps, and plant cells that are intact in plants or parts of plants, such as pollen, ovules, embryos, protoplasts, meristematic cells, callus, leaves, anthers, cotyledons, hypocotyl, pistils, roots, root tips, seeds, flowers, petiole, shoot, or stems and the like.

Progeny. As used herein, the descendants of one or more of the parental lines and includes an F₁ basil plant produced from the cross of two basil plants where at least one plant includes a basil plant disclosed herein and progeny further includes, but is not limited to, subsequent F₂, F₃, F₄, F₅, F₆, F₇, F₈, F₉, and F₁₀ generational crosses with the recurrent parental line.

Single Gene Converted (Conversion). Single gene converted (conversion) plants refers to plants which are developed by a plant breeding technique called backcrossing wherein essentially all of the desired morphological and physiological characteristics of a variety are recovered via the backcrossing technique in addition to the single gene(s) transferred into the variety via the initial cross or via genetic engineering.

SNP. As used herein, the term “SNP” shall refer to a single nucleotide polymorphism.

### DETAILED DESCRIPTION

To date, there have been no known Ocimum basilicum sweet basil type varieties that are resistant to Peronospora belbahrii. Peronospora belbahrii is carried on seed and the only current way to treat infected plants is through the use of fungicides. Thus, tolerance to Peronospora belbahrii for sweet basil is a highly valuable commercial trait.

### Examples

Protocol for Evaluating Plants for Degrees of Responses to Peronospora belbahrii

Basil seedling response to Peronospora belbahrii was screened under environmental conditions which allow disease development. This is a method that is used in identifying genetic variation for resistance or susceptibility to the pathogen. Seedlings were screened using a scoring system to evaluate the level of disease development. First, seeds were sown into plug trays and placed in conditions to promote germination. Plants remained in the plug trays throughout the evaluation. ‘Nufar’ was used as a susceptible control and ‘Blue Spice’ was used as a resistant control. A 192-plug tray was used and in each plug tray, there were 16 plugs each of the above control. When the plants developed two true leaves, they were inoculated with Peronospora belbahrii spores harvested from leaves on which sporulation had recently taken place. To make the inoculation solution, the harvested leaves were placed in a container with distilled water and agitated. The inoculum solution was adjusted to a concentration of 50,000 spores per milliliter of water using a hemocytometer. The inoculum was applied to the upper surface of the leaves using a hand sprayer. The inoculated plants in the plug trays were placed in a closed plastic chamber for the next 16 hours. The conditions in the chamber were total darkness, a temperature range of 19 to 21 degrees Celsius, relative humidity ranges between 85% to 90% for the first 4 hours, and for hours 4 through 16, the relative humidity was greater than 90%. Relative humidity was recorded with a data logger inside the plastic chamber. After 16 hours in the chamber, the plants were removed and placed in full sunlight. The plants remained outside the nursery under natural day length and night length. ‘Nufar’ control plants consistently developed areas of chlorosis on the leaves by 6 days, indicating progression of disease development within the leaf after infection of Peronospora belbahrii. The plant material, still in the original plug trays, was placed into a plastic chamber for about 16 hours under the protocol mentioned above. After the 16 hours, the plants were removed and rated for disease development using the scoring system discussed below.

Scoring of Responses to Basil Downy Mildew, Peronospora belbahrii

Tolerance to Peronospora belbahrii was scored using the following system, where “resistant” means that the seedling leaves do not express chlorotic symptoms or sporulation of the pathogen. “High tolerance/no sporulation” means that seedlings develop the disease symptoms of chlorosis, with no sporulation observed. “High tolerance” means seedling leaves still develop symptoms of chlorosis, but there is low sporulation on no more than 10% of the leaf area. The sporangiophores are not well-branched, and the color of the sporangia are light-gray. High tolerance is more clearly observed under a stereoscope. “Tolerance” means seedling leaves which show symptoms of chlorosis, with sporulation areas ranging up to 20% of the area of the leaves, but not more than 30% of the area of the leaves. Sporangiophores are well-developed, branched, and the color of the sporangia are dark gray-black. “Susceptible” means seedling leaves express chlorosis and necrotic symptoms. Sporangiophores are well-developed and branched. Sporulation is present in more than 30% of the leaf area. Sporangia are a dark color and can be observed in mass with the naked eye. Sporangiophore groups are very visible without the use of a magnifying glass and spread very uniformly over the chlorotic areas of the leaves.

Origin of Highly Tolerant Ocimum basilicum to Peronospora belbahrii, Basil Downy Mildew

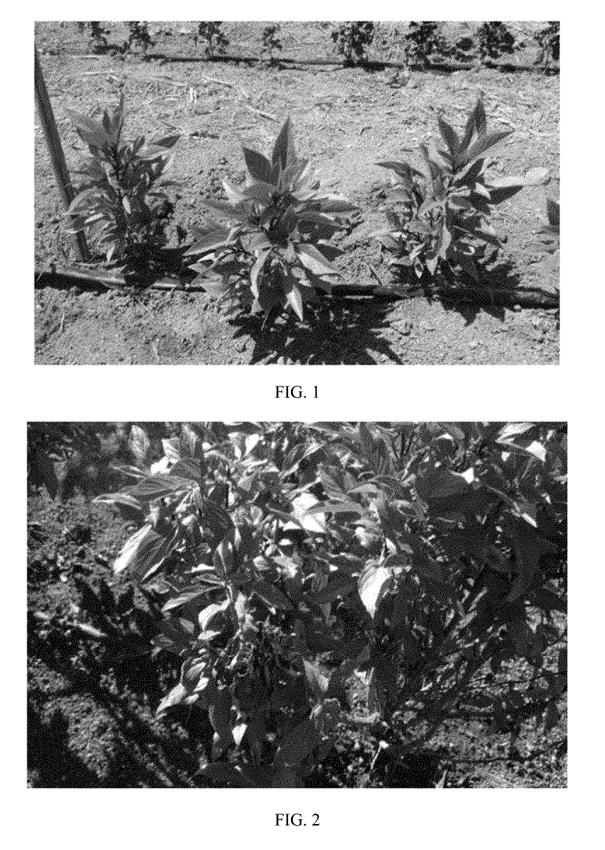

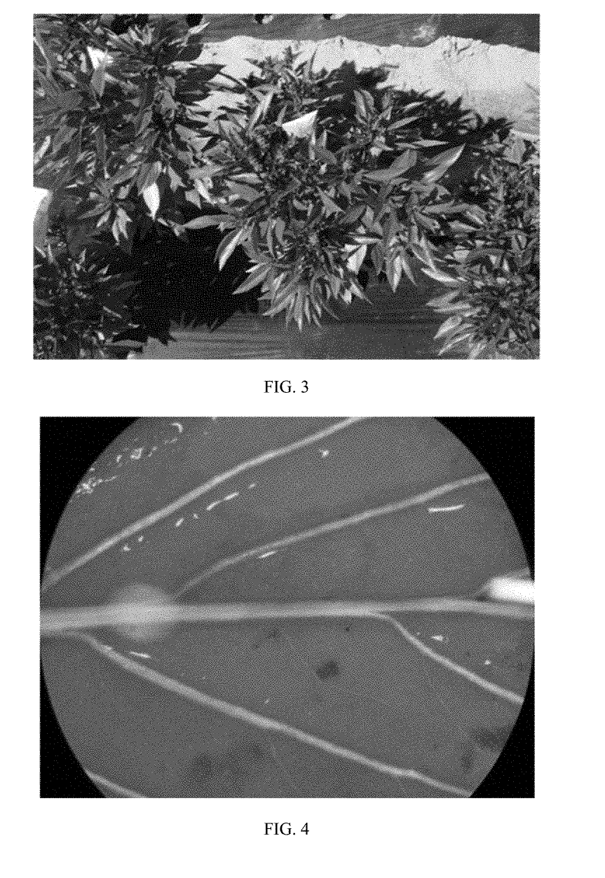

Twenty-four seeds of the female/seed parent, a basil line B1020 that was Peronospora belbahrii tolerant, were sown on Jan. 19, 2015 into plug flats in San Jose Del Cabo, Mexico. Young plants were transplanted on Feb. 18, 2015 into a research field for the purpose of continued observation. The characteristics of the seed parent included plants having purple stems and elongated green leaves with a strong anise flavor (please see FIG. 1). Stem diameter was less than that of the widely known commercial cultivar ‘Nufar’. Seeds were black in color and similar to length when compared to ‘Nufar’, but were more narrow. The flowering structure of the plants is shown in FIG. 3. The plants were susceptible to Fusarium oxysporum f. basilicum, a fungal disease which causes Fusarium wilt (please see FIG. 2). The female B1020 plants were tolerant to Basil Downy Mildew with small areas of leaves with brown discoloration (please see FIG. 4). Areas of brown discoloration on leaves are observed on this plant under environmental conditions of severe Basil Downy Mildew disease pressure. The brown discoloration possibly indicates a hypersensitive response by the plant, restricting the development of the disease from the Basil Downy Mildew pathogen within the leaf (please see FIG. 4).

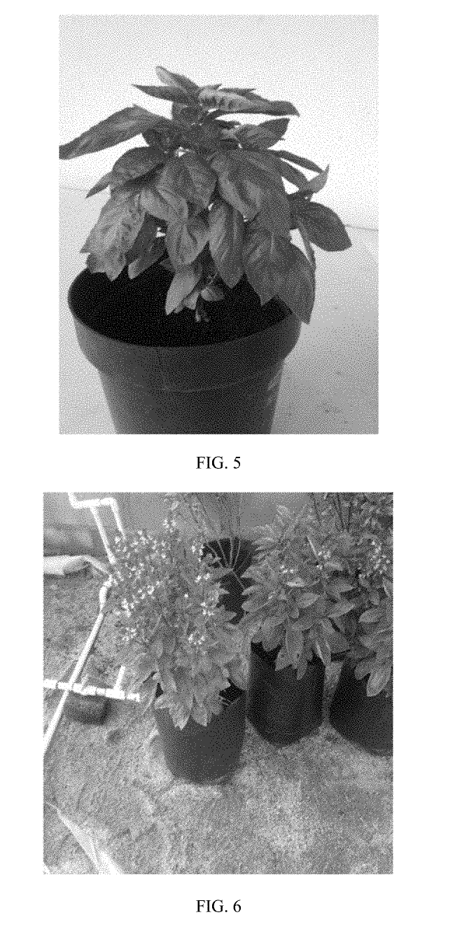

Seed derived from open-pollination of O. basilicum line B1020, in a field that included a wide range of O. basilicum germplasm conducted in San Jose Del Cabo, Mexico in May 2015, were harvested from the open-pollination. The F₁ seed from the open-pollination were sown on Jun. 8, 2015 and the resulting population varied for level of susceptibility and tolerance to Peronospora belbahrii, leaf shape and color, degree of leaf glossiness, and plant vigor. From this F₁ population, plants were selected as seedlings and transplanted into pots. Selection was for level of tolerance to Peronospora belbahrii, Genovese basil leaf type, glossy leaf, and high plant vigor. A single plant was selected and designated as B1020 OP1 (please see FIG. 5). This plant was scored as tolerant to Peronospora belbharii. Additional plant characteristics of the F₁ selection were purple stem and flower, less concentrated anise concentration flavor of the leaves, and erect, single spiked flower structures. The original female parent, in contrast, had a multiple branching flower structure. Thus, it is hypothesized that, due to the characteristics of B1020 OP1, the pollen parent must have been a sweet basil type which was included in the open-pollination of the mixture.

B1020 OP1 F₁ was subsequently moved to a closed shade house in June 2015 for continued growing. The closed shade structure prevented entrance of pollinating insects (FIG. 6). The plant remained in the closed shade structure through flowering and self-pollination until seed maturity. Harvest of the F₂ seed started in October 2015 and was completed in January 2016. The F₂ seed was sown in February 2016 and plants were grown in plug trays until susceptible plants were identified through screening described herein, and discarded before transplanting the remaining plants to the field. These remaining plants varied in tolerance classes, which included tolerant, highly tolerant and highly tolerant/no sporulation. F₂ segregants in the HT and HT no sporulation class exceeded the level of tolerance expressed by female parent B1020 which was classified as tolerant as well as B1020 OP1.

In March 2016, seedlings were inoculated and then rated using the protocol below and scoring system above. As shown below in Table 1, the F₂ population consisted of 384 plants, where 43 plants expressed some level of tolerance. Of the F₂ population, 341 plants were susceptible, 5 plants expressed high tolerance/no sporulation, 9 plants exhibited high tolerance, and 29 plants exhibited tolerance. 9 F₂ plants were selected from this population and were named 165303-1 (no sporulation), 165303-2 (no sporulation), 165303-3 (no sporulation), 165303-4 (no sporulation), 165303-5 (no sporulation), 165303-6 or BHT-1 (highly tolerant), 165303-7 or BHT-2 (tolerant), 165303-9 (highly tolerant), and 165303-10 (highly tolerant). Selections BHT-1 and BHT-2 were subsequently chosen from the F₂ population based on their response to Peronospora belbahrii as well as desirable market characteristics, such as aroma, flavor, completely green stem and leaves, absence of pubescence, and plant vigor.

TABLE 1
F₂ Segregants of B1020 OP1

| Tolerance score | | | |
|---|---|---|---|
| to Peronospora | No. of | % of total | |
| belbahrii | seedlings | offspring | Plant selections |
| Susceptible | 341 | 88.8% | None |
| High tolerance/ | 5 | 1.3% | 165303-1, 165303-2, 165303-3, |
| no sporulation | | | 165303-4, 165303-5 |
| High tolerance | 9 | 2.3% | 165303-6 or BHT-1, 165303-9, |
| | | | 165303-10 |
| Tolerant | 29 | 7.5% | 165303-7 or BHT-2 |
| Some | 43/384 | 11.2% | |
| tolerance/Total | | | |
| F₂ offspring | | | |

Determination of Genetic Mechanism and Inheritance of Higher Tolerance to Peronospora belbahrii

All of the 43 plants from the previously mentioned F₂ population which were shown to express some level of tolerance to Peronospora belbahrii were screened again using the protocol outlined above and the responses were verified as being the same as the initial screening.

BHT-1, a selection that was observed as highly tolerant to Peronospora belbahrii, was self-pollinated. F₃ seeds were collected and sown in a plug tray and screened for response to Peronospora belbahrii. Out of the 367 plants that were sown in the field, 26 plants (7.08%) expressed tolerance to Peronospora belbahrii, 55 (14.99%) expressed no sporulation, 108 (29.43%) expressed high tolerance to Peronospora belbahrii, and 178 (48.50%) expressed susceptibility to Peronospora belbahrii.

TABLE 2
F₃ Segregants of BHT-1

| Tolerance score to Peronospora | No. of | % of total |
|---|---|---|
| belbahrii | seedlings | offspring |
| Susceptible | 178 | 48.50% |
| High tolerance/no sporulation | 55 | 14.99% |
| High tolerance | 108 | 29.43% |
| Tolerant | 26 | 7.08% |
| Some tolerance/Total F₃ offspring | 189/367 | 51.50% |

Looking at the F₂ and F₃ population, the data shows that tolerance to Peronospora belbahrii is heritable, and most likely polygenic and quantitative. It is possible that there is a dominant gene and at least one homozygous recessive gene that affects tolerance to Peronospora belbahrii. A recent publication by Pyne R. et al., compiled a linkage map of 42 expressed sequence tag (EST) simple sequence repeats (SSR) and 1,847 SNPs. One major quantitative trait loci (QTL) and two minor QTL were associated with response to downy mildew in their Mill X SB22 mapping population. (Pyne R, Honig J, Vaiciunas J, Koroch A, Wyenandt C, Bonos S, et al. (2017) A first linkage map and downy mildew resistance QTL discovery for sweet basil (Ocimum basilicum) facilitated by double digestion restriction site associated DNA sequencing (ddRADseq). PLoS ONE 12(9):e0184319).

The basic chromosome number of O. basilicum is considered to be X=12. Total chromosome number is 2n=48. Please see Carovic-Stanko, K., Liber Z., Besendorfer V., Javornik B., Bohanec B., Kolak I. Satovic Z., 2009. Genetic relations among basil taxa (Ocimum L.) based on molecular markers, nuclear DNA content, and chromosome number. Plant Syst Evol. and R. Omidbaigi, M. Mirzaee, M. E. Hassani, M. Sedghi Moghadam 2009, Induction and identification of polyploidy in basil (Ocimum basilicum L.) medicinal plant by colchicine treatment. International Journal of Plant Production.

Through breeding and laboratory techniques well-known in the art, sweet basil plants can be produced as a tetrapoloid or diploid. Additionally, the high tolerance to Peronospora belbahrii trait can be transferred stably and predictably across different basil species, and different genetic backgrounds.

### 2. The Characteristics of BHT-1

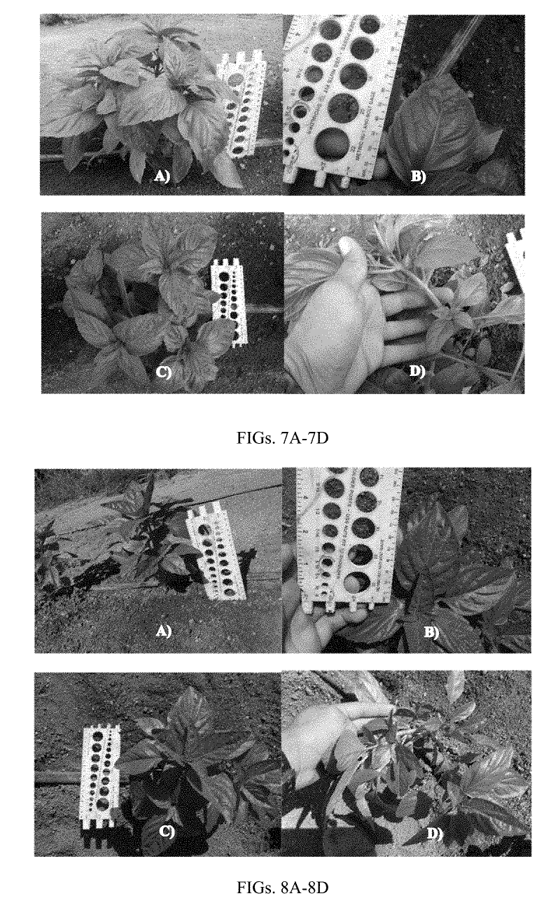

Table 3 shows the characteristics of the selection BHT-1. FIGS. 7A-7D show the overall plant habit, the length of the mature leaf, the view from above the plant, and the internodes and stems of the selection.

TABLE 3
Characteristics of BHT-1

| Characteristic | Expression |
|---|---|
| Plant habit | Erect |
| Pubescence | Minor |
| Leaf shape | Broad ovate |
| Leaf length | Medium; 80.mm |
| Leaf width | Broad; 60 mm |
| Serration of leaf margin | Medium |
| Flowering description | Single flowering structure; green calyx which |
| | develops white flowers |
| Flavor | Sweet basil with minor note of clove |
| Aroma | Sweet basil with minor note of clove |

### 3. The Characteristics of BHT-2

Table 2 shows the characteristics of the selection BHT-2. FIGS. 8A-8D show the overall plant habit, the length of the mature leaf, the view from above the plant, and the internodes and stems of the selection.

TABLE 1
Characteristics of BHT-2

| Characteristic | Expression |
|---|---|
| Plant habit | Erect |
| Pubescence | Minor |
| Leaf shape | Ovate |
| Leaf length | Medium; 80.mm |
| Leaf width | Broad; 60 mm |
| Serration of leaf margin | Low and shallow |
| Flowering description | Single flowering structure; green calyx which |
| | develops white flowers |
| Flavor | Sweet basil with minor note of citrus |
| Aroma | Sweet basil with minor note of citrus |

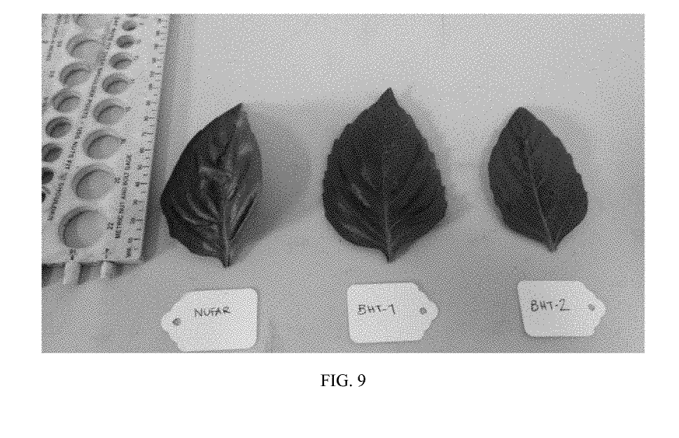

FIG. 9 shows a comparison between the leaf appearance from left to right, the commercial variety ‘Nufar’, BHT-1, and BHT-2. Note the differences in leaf appearance where the BHT-1 and BHT-2 selections have a serration pattern and a flatter leaf appearance than ‘Nufar’.

4. Discovery of SNPs Markers for Tolerance to Peronospora belbahrii

SNP data were obtained using whole genome sequencing of sweet basil. The first phase involved collecting leaf samples from about 300 F₃ plants and DNA was bulked from 24 plants in 4 groups: a) resistant F₃ lines, b) susceptible F₃ lines, c) resistant parent and d) susceptible parent. DNA was extracted and bulked whole genome sequencing was performed in order to find SNP markers segregating in the pools, as well as markers associated with the pools (present in one pool and absent in another) and markers were further to those where having fixed alleles.

SNP marker development for Downy Mildew tolerance was obtained using a modified Bulk Segregant Analysis (BSA) approach and developing a linked marker for the Downy Mildew gene(s) in order to apply the results in a marker-assisted selection scheme. BSA is well-known in the art and followed the protocol as outlined in Michelmore, R. W. et al., “Identification of markers linked to disease-resistance genes by bulked segregant analysis: A rapid method to detect markers in specific genomic regions by using segregating populations” Proc. Natl. Acad. Sci. USA 88:9828-9832 (November 1991) Genetics. To obtain the SNP sequences, which is well-known in the art, DNA samples were processed to sequencing compatible libraries. Samples were sequenced on the Illumina HiSeq X PE150 and data was transferred as raw FASTQ as well as SNP data in Excel files.

5. Crosses of the Sweet Basil of the Present Application with Other Basil Types—Interspecific Crosses of Basil

As shown by the breeding history and data, the higher tolerance to Peronospora belbahrii characteristic is a heritable trait and can be bred into other Ocimum plants. Interspecific crosses in the genus Ocimum are well-known in the art. Please see Vieira, Roberto F., “Genetic Diversity of Basil (Ocimum spp.) based on RAPD Markers”, J. Amer. Soc. Hort. Sci., 128(1): 94-99 (2003).

For example, a representative sample of seed of sweet basil plant 165303-6-B (also known as BHT-1, obtained in the F₂) was deposited under NCIMB No. 42726, may be bred with at least one other Ocimum species such as O. americanum, O. citriodorum, O. gratissimum, and O. tenuiflorum to produce at least one interspecific hybrid seed having some level of tolerance to Peronospora belbahrii.

### Further Embodiments

Characterization of the sweet basil plant complemention of alleles at different loci is indicated by data that some F₂ progeny express a level of high tolerance to Peronospora belbharii which is beyond that expressed by any other known O. basilicum using complementation assays

In addition to the sequences provided herein, the recessive alleles responsible for higher tolerance levels of sweet basil to Peronospora belbharii characteristic of the present application can be identified using complementation assays, which are well-known in the art. See for example, Griffiths et al. “An Introduction to Genetic Analysis” 7^{th} Edition. W.H. Freeman (2000), explaining how a mutant condition that is determined by a Z and w alleles can be determined.

Breeding with the Sweet Basil Plants of the Present Application

The goal of plant breeding is to develop new, unique and superior varieties and hybrids. The breeder initially selects and crosses two or more parental lines, followed by repeated selfing and selection, producing many new genetic combinations. The breeder can theoretically generate billions of different genetic combinations via crossing, selection, selfing and mutations. Therefore, a breeder will never develop the same variety genetically and having the same traits from the exact same parents.

Each year, the plant breeder selects the germplasm to advance to the next generation. This germplasm is grown under unique and different geographical, climatic and soil conditions and further selections are then made during and at the end of the growing season. The varieties that are developed are unpredictable because the breeder's selection occurs in unique environments with no control at the DNA level, and with millions of different possible genetic combinations being generated. A breeder of ordinary skill in the art cannot predict the final resulting lines he develops, except possibly in a very gross and general fashion. The same breeder cannot produce the same variety twice by using the same original parents and the same selection techniques. This unpredictability results in the expenditure of large amounts of research monies to develop superior new basil varieties.

Breeding programs combine desirable traits from two or more varieties or various broad-based sources into breeding pools from which varieties are developed by selfing and selection of desired phenotypes. Pedigree breeding is used commonly for the improvement of self-pollinating plants. Two parents that possess favorable, complementary traits are crossed to produce an F₁. An F₂ population is produced by selfing one or several F₁s. Selection of the best individuals may begin in the F₂ population; then, beginning in the F₃, the best individuals in the best families are selected. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as new varieties.

### Using the Sweet Basil Plants of the Present Application to Develop Other Basil Plants

Basil, such as the sweet basil plants of the present application are developed for sales in the food, medicinal, and consumer market. However, said basil plants can also provide a source of breeding material that may be used to develop new basil plants and varieties. Plant breeding techniques known in the art and used in a basil plant breeding program include, but are not limited to, recurrent selection, mass selection, bulk selection, hybridization, mass selection, backcrossing, pedigree breeding, open-pollination breeding, restriction fragment length polymorphism enhanced selection, genetic marker enhanced selection, making double haploids, mutagenesis and transformation. Often combinations of these techniques are used. There are many analytical methods available to evaluate a new variety. The oldest and most traditional method of analysis is the observation of phenotypic traits, but genotypic analysis may also be used.

### Additional Breeding Methods

Any plants produced using the basil plants disclosed in the present application as at least one parent are also an embodiment. These methods are well-known in the art and some of the more commonly used breeding methods are described herein. Descriptions of breeding methods can be found in one of several reference books (e.g., Allard, “Principles of Plant Breeding” (1999); Ribeiro, P. G. “Breeding for Chilling Tolerance in Basil (Ocimum basilicum L.)” Rutgers, the State University of New Jersey. 154 pages. (2007).

Breeding steps that may be used in the basil plant breeding program can include for example, pedigree breeding, backcrossing, mutation breeding, and recurrent selection. In conjunction with these steps, techniques such as RFLP-enhanced selection, genetic marker enhanced selection (for example, SSR markers, SNP markers), gene editing, and the making of double haploids may be utilized.

One method for detection of SNPs in DNA samples is by use of PCR in combination with fluorescent probes for the polymorphism, as described in Livak et al., 1995 and U.S. Pat. No. 5,604,099, incorporated herein by reference. Briefly, two probe oligonucleotides, one of which anneals to the SNP site and the other which anneals to the wild type sequence, are synthesized. It is preferable that the site of the SNP be near the 5′ terminus of the probe oligonucleotides. Each probe is then labeled on the 3′ end with a non-fluorescent quencher and a minor groove binding moiety which lower background fluorescence and lower the Tₘ of the oligonucleotide, respectively. The 5′ ends of each probe are labeled with a different fluorescent dye wherein fluorescence is dependent upon the dye being cleaved from the probe. Some non-limiting examples of such dyes include VIC and 6-FAM DNA suspected of comprising a given SNP is then subjected to PCR using a polymerase with 5′-3′ exonuclease activity and flanking primers. PCR is performed in the presence of both probe oligonucleotides. If the probe is bound to a complimentary sequence in the test DNA then exonuclease activity of the polymerase releases a fluorescent label activating its fluorescent activity. Therefore, test DNA that contains only wild type sequence will exhibit fluorescence associated with the label on the wild type probe. On the other hand, DNA containing only the SNP sequence will have fluorescent activity from the label on the SNP probe. However, in the case that the DNA is from heterogeneous sources, significant fluorescence of both labels will be observed. This type of indirect genotyping at known SNP sites enables high throughput, inexpensive screening of DNA samples.

Restriction fragment length polymorphisms (RFLPs) are genetic differences detectable by DNA fragment lengths, typically revealed by agarose gel electrophoresis, after restriction endonuclease digestion of DNA. There are large numbers of restriction endonucleases available, characterized by their nucleotide cleavage sites and their source, e.g., EcoRI. RFLPs result from both single base pair polymorphisms within restriction site sequences and measurable insertions or deletions within a given restriction fragment. RFLPs are easy and relatively inexpensive to generate (require a cloned DNA, but no sequence) and are co-dominant. RFLPs have the disadvantage of being labor-intensive in the typing stage, although this can be alleviated to some extent by multiplexing many of the tasks and reutilization of blots.

One of skill in the art would recognize that many types of molecular markers are useful as tools to monitor genetic inheritance and are not limited to RFLPs, SSRs and SNPs, and one of skill would also understand that a variety of detection methods may be employed to track the various molecular markers. One skilled in the art would also recognize that markers of different types may be used for mapping, especially as technology evolves and new types of markers and means for identification are identified.

### Pedigree Breeding

Pedigree breeding starts with the crossing of two genotypes, such as the basil plant of the instant application and another different basil plant having one or more desirable characteristics that is lacking or which complements the phenotype of the basil plant of the present application. If the two original parents do not provide all the desired characteristics, other sources can be included in the breeding population. In the pedigree method, superior plants are selfed and selected in successive filial generations. In the succeeding filial generations, the heterozygous condition gives way to homogeneous varieties as a result of self-pollination and selection. Typically, in the pedigree method of breeding, five or more successive filial generations of selfing and selection is practiced: F₁ to F₂; F₂ to F₃; F₃ to F₄; F₄ to F₅; etc. After a sufficient amount of inbreeding, successive filial generations will serve to increase seed of the developed variety. Preferably, the developed variety comprises homozygous alleles at about 95% or more of its loci.

### Backcross Breeding

Backcross breeding has been used to transfer genes for a simply inherited, highly heritable trait into a desirable homozygous variety or inbred line which is the recurrent parent. The source of the trait to be transferred is called the donor parent. After the initial cross, individuals possessing the phenotype of the donor parent are selected and repeatedly crossed (backcrossed) to the recurrent parent. The resulting plant is expected to have the attributes of the recurrent parent and the desirable trait transferred from the donor parent.

In addition to being used to create a backcross conversion, backcrossing can also be used in combination with pedigree breeding. As discussed previously, backcrossing can be used to transfer one or more specifically desirable traits from one variety, the donor parent, to a developed variety called the recurrent parent, which has overall good commercial characteristics yet lacks that desirable trait or traits. However, the same procedure can be used to move the progeny toward the genotype of the recurrent parent, but at the same time retain many components of the nonrecurrent parent by stopping the backcrossing at an early stage and proceeding with selfing and selection. For example, a basil plant may be crossed with another variety to produce a first-generation progeny plant. The first-generation progeny plant may then be backcrossed to one of its parent varieties to create a BC₁ or BC₂. Progeny are selfed and selected so that the newly developed variety has many of the attributes of the recurrent parent and yet several of the desired attributes of the nonrecurrent parent. This approach leverages the value and strengths of the recurrent parent for use in new basil varieties.

Therefore, another embodiment is a method of making a backcross conversion of the basil plant of the present application, comprising the steps of crossing a plant of the basil plant of the present application with a donor plant comprising a desired trait, selecting an F₁ progeny plant comprising the desired trait, and backcrossing the selected F₁ progeny plant to a plant of the basil plant of the present application. This method may further comprise the step of obtaining a molecular marker profile of the basil plants of the present application and using the molecular marker profile to select for a progeny plant with the desired trait and the molecular marker profile of the basil plant of the present application.

### Recurrent Selection and Mass Selection

Recurrent selection is a method used in a plant breeding program to improve a population of plants. The basil plants of the present application are suitable for use in a recurrent selection program. The method entails individual plants cross-pollinating with each other to form progeny. The progeny are grown and the superior progeny selected by any number of selection methods, which include individual plant, half-sib progeny, full-sib progeny, and selfed progeny. The selected progeny are cross-pollinated with each other to form progeny for another population. This population is planted and again superior plants are selected to cross-pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use.

Mass selection is a useful technique when used in conjunction with molecular marker enhanced selection. In mass selection, seeds from individuals are selected based on phenotype or genotype. These selected seeds are then bulked and used to grow the next generation. Bulk selection requires growing a population of plants in a bulk plot, allowing the plants to self-pollinate, harvesting the seed in bulk, and then using a sample of the seed harvested in bulk to plant the next generation. Also, instead of self-pollination, directed pollination could be used as part of the breeding program.

Mass and recurrent selections can be used to improve populations of either self- or cross-pollinating plants. A genetically variable population of heterozygous individuals is either identified, or created, by intercrossing several different parents. The best plants are selected based on individual superiority, outstanding progeny, or excellent combining ability. The selected plants are intercrossed to produce a new population in which further cycles of selection are continued.

### Mutation Breeding

Mutation breeding is another method of introducing new traits into the basil plants of the present application. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, ionizing radiation, such as X-rays, Gamma rays (e.g., cobalt 60 or cesium 137), neutrons, (product of nuclear fission by uranium 235 in an atomic reactor), Beta radiation (emitted from radioisotopes such as phosphorus 32 or carbon 14), or ultraviolet radiation (preferably from 2500 to 2900 nm); chemical mutagens (such as base analogues (5-bromo-uracil)), related compounds (8-ethoxy caffeine), antibiotics (streptonigrin), alkylating agents (sulfur mustards, nitrogen mustards, epoxides, ethylenamines, sulfates, sulfonates such as ethyl methanesulfonate, sulfones, lactones), sodium azide, hydroxylamine, nitrous acid, methylnitrilsourea, or acridines; TILLING (targeting induced local lesions in genomes), where mutation is induced by chemical mutagens and mutagenesis is accompanied by the isolation of chromosomal DNA from every mutated plant line or seed and screening of the population of the seed or plants is performed at the DNA level using advanced molecular techniques; zinc finger nucleases. Once a desired trait is observed through mutagenesis the trait may then be incorporated into existing germplasm by traditional breeding techniques. Details of mutation breeding can be found in Sikora, Per, et al., “Mutagenesis as a Tool in Plant Genetics, Functional Genomics, and Breeding” International Journal of Plant Genomics. 2011 (2011); 13 pages; Petilino, Joseph F. “Genome editing in plants via designed zinc finger nucleases” In Vitro Cell Dev Biol Plant. 51(1): pp. 1-8 (2015); and Daboussi, Fayza, et al. “Engineering Meganuclease for Precise Plant Genome Modification” in Advances in New Technology for Targeted Modification of Plant Genomes. Springer Science+Business. pp 21-38 (2015). In addition, mutations created in other basil plants may be used to produce a backcross conversion of the basil plants of the present application that comprises such mutation.

### Gene Editing Using CRISPR

Targeted gene editing can be done using CRISPR/Cas9 technology (Saunders & Joung, Nature Biotechnology, 32, 347-355, 2014). CRISPR is a type of genome editing system that stands for Clustered Regularly Interspaced Short Palindromic Repeats. This system and CRISPR-associated (Cas) genes enable organisms, such as select bacteria and archaea, to respond to and eliminate invading genetic material. Ishino, Y., et al. J. Bacteriol. 169, 5429-5433 (1987). These repeats were known as early as the 1980s in E. coli, but Barrangou and colleagues demonstrated that S. thermophilus can acquire resistance against a bacteriophage by integrating a fragment of a genome of an infectious virus into its CRISPR locus. Barrangou, R., et al. Science 315, 1709-1712 (2007). Many plants have already been modified using the CRISPR system. See for example, U.S. Application Publication No. WO2014068346 (György et al., Identification of a Xanthomonas euvesicatoria resistance gene from pepper (Capsicum annuum) and method for generating plants with resistance); Martinelli, F. et al., “Proposal of a Genome Editing System for Genetic Resistance to Tomato Spotted Wilt Virus” American Journal of Applied Sciences 2014; and Noman, A. et al., “CRISPR-Cas9: Tool for Qualitative and Quantitative Plant Genome Editing” Frontiers in Plant Science Vol. 7 Nov. 2016.

Gene editing can also be done using crRNA-guided surveillance systems for gene editing. Additional information about crRNA-guided surveillance complex systems for gene editing can be found in the following documents, which are incorporated by reference in their entirety: U.S. Application Publication No. 2010/0076057 (Sontheimer et al., Target DNA Interference with crRNA); U.S. Application Publication No. 2014/0179006 (Feng, CRISPR-CAS Component Systems, Methods, and Compositions for Sequence Manipulation); U.S. Application Publication No. 2014/0294773 (Brouns et al., Modified Cascade Ribonucleoproteins and Uses Thereof); Sorek et al., Annu. Rev. Biochem. 82:273-266, 2013; and Wang, S. et al., Plant Cell Rep (2015) 34: 1473-1476. Therefore, it is another embodiment to use the CRISPR system on the basil plants of the present application to modify traits and resistances or tolerances to pests, herbicides, diseases, and viruses.

### Additional Methods for Detecting SNPs for Marker Assisted Breeding

In addition to the direct or indirect sequencing of the site and the probes described above, the SNPs disclosed herein may also be detected by a variety of effective methods well known in the art including those disclosed in U.S. Pat. Nos. 5,468,613 and 5,217,863; 5,210,015; 5,876,930; 6,030,787 6,004,744; 6,013,431; 5,595,890; 5,762,876; 5,945,283; 5,468,613; 6,090,558; 5,800,944 and 5,616,464. In particular, polymorphisms in DNA sequences can be detected by hybridization to allele-specific oligonucleotide (ASO) probes as disclosed in U.S. Pat. Nos. 5,468,613 and 5,217,863. The nucleotide sequence of an ASO probe is designed to form either a perfectly matched hybrid or to contain a mismatched base pair at the site of the variable nucleotide residues. The distinction between a matched and a mismatched hybrid is based on differences in the thermal stability of the hybrids in the conditions used during hybridization or washing, differences in the stability of the hybrids analyzed by denaturing gradient electrophoresis or chemical cleavage at the site of the mismatch.

If a SNP creates or destroys a restriction endonuclease cleavage site, it will alter the size or profile of the DNA fragments that are generated by digestion with that restriction endonuclease. As such, plants that possess a variant sequence can be distinguished from those having the original sequence by restriction fragment analysis. SNPs that can be identified in this manner are termed “restriction fragment length polymorphisms” (“RFLPs”). RFLPs have been widely used in human and plant genetic analyses (Glassberg, UK Patent Application 2135774; Skolnick et al., Cytogen. Cell Genet. 32:58-67 (1982); Botstein et al., Ann. J. Hum. Genet. 32:314-331 (1980); Fischer et al., PCT Application WO 90/13668; Uhlen, PCT Application WO 90/11369.

An alternative method of determining SNPs is based on cleaved amplified polymorphic sequences (CAPS) (Konieczny, A. and F. M. Ausubel, Plant J. 4:403-410 (1993); Lyamichev et al., Science 260:778-783 (1993). A modified version of CAPs, known as dCAPs, is a technique for detection of Single Nucleotide Polymorphisms (SNPs). The dCAPS technique introduces or destroys a restriction enzyme recognition sites by using primers that containing one or more mismatches to the template DNA. The PCR product modified in this manner is then subjected to restriction enzyme digestion and the presence or absence of the SNP is determined by the resulting restriction pattern. This technique is useful for genotyping known mutations and genetic mapping of isolated DNAs (Neff M M, Neff J D, Chory J, Pepper A E. dCAPS, a simple technique for the genetic analysis of single nucleotide polymorphisms: experimental applications in Arabidopsis thaliana genetics. Plant J. 1998 May; 14(3):387-92).

SNPs can also be identified by single strand conformation polymorphism (SSCP) analysis. The SSCP technique is a method capable of identifying most sequence variations in a single strand of DNA, typically between 150 and 250 nucleotides in length (Elles, Methods in Molecular Medicine: Molecular Diagnosis of Genetic Diseases, Humana Press (1996); Orita et al., Genomics 5:874-879 (1989). Under denaturing conditions a single strand of DNA will adopt a conformation that is uniquely dependent on its sequence. This conformation usually will be different even if only a single base is changed. Most conformations have been reported to alter the physical configuration or size sufficiently to be detectable by electrophoresis. A number of protocols have been described for SSCP including, but not limited to Lee et al., Anal. Biochem. 205:289-293 (1992); Suzuki et al., Anal. Biochem. 192:82-84 (1991); Lo et al., Nucleic Acids Research 20:1005-1009 (1992); Sarkar et al., Genomics 13:441-443 (1992).

SNPs may also be detected using a DNA fingerprinting technique called amplified fragment length polymorphism (AFLP), which is based on the selective PCR amplification of restriction fragments from a total digest of genomic DNA to profile that DNA. Vos et al., Nucleic Acids Res. 23:4407-4414 (1995). This method allows for the specific co-amplification of many restriction fragments, which can be analyzed without knowledge of the nucleic acid sequence. AFLP employs basically three steps. Initially, a sample of genomic DNA is cut with restriction enzymes and oligonucleotide adapters are ligated to the restriction fragments of the DNA. The restriction fragments are then amplified using PCR by using the adapter and restriction sequence as target sites for primer annealing. The selective amplification is achieved by the use of primers that extend into the restriction fragments, amplifying only those fragments in which the primer extensions match the nucleotide flanking the restriction sites. These amplified fragments are then visualized on a denaturing polyacrylamide gel (Beismann et al., Mol. Ecol. 6:989-993 (1997); Janssen et al., Int. J. Syst. Bacteriol 47:1179-1187 (1997); Huys et al., Int. J. Syst. Bacteriol. 47:1165-1171 (1997); McCouch et al., Plant Mol. Biol. 35:89-99 (1997); Nandi et al., Mol. Gen. Genet. 255:1-8 (1997); Cho et al. Genome 39:373-378 (1996); Simons et al., Genomics 44:61-70 (1997); Cnops et al., Mol. Gen. Genet. 253:32-41 (1996); Thomas et al., Plant J. 8:785-794 (1995).

SNPs may also be detected using random amplified polymorphic DNA (RAPD) (Williams et al., Nucl. Acids Res. 18:6531-6535 (1990).

SNPs can be detected by methods as disclosed in U.S. Pat. Nos. 5,210,015; 5,876,930 and 6,030,787 in which an oligonucleotide probe having reporter and quencher molecules is hybridized to a target polynucleotide. The probe is degraded by 5′→3′ exonuclease activity of a nucleic acid polymerase.

SNPs can also be detected by labelled base extension methods as disclosed in U.S. Pat. Nos. 6,004,744; 6,013,431; 5,595,890; 5,762,876; and 5,945,283. These methods are based on primer extension and incorporation of detectable nucleoside triphosphates. The primer is designed to anneal to the sequence immediately adjacent to the variable nucleotide which can be can be detected after incorporation of as few as one labelled nucleoside triphosphate. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labelled sequence-specific oligonucleotide probe

Other methods for identifying and detecting SNPs in addition to those described above include the use of restriction enzymes (Botstein et al., Am. J. Hum. Genet. 32:314-331 (1980); and Konieczny and Ausubel, Plant J. 4:403-410 (1993)), enzymatic and chemical mismatch assays (Myers et al., Nature 313:495-498 (1985)), allele-specific PCR (Newton et al., Nucl. Acids Res. 17:2503-2516 (1989); and Wu et al., Proc. Natl. Acad. Sci. USA 86:2757-2760 (1989)), ligase chain reaction (Barany, Proc. Natl. Acad. Sci. USA 88:189-193 (1991)), single-strand conformation polymorphismanalysis (Labrune et al., Am. J. Hum. Genet. 48: 1115-1120 (1991)), single base primer extension (Kuppuswamy et al., Proc. Natl. Acad. Sci. USA 88:1143-1147 (1991); and Goelet, U.S. Pat. Nos. 6,004,744 and 5,888,819, solid-phase ELISA-based oligonucleotide ligation assays (Nikiforov et al., Nucl. Acids Res. 22:4167-4175 (1994)), dideoxy fingerprinting (Sarkar et al., Genomics 13:441-443 (1992)), oligonucleotide fluorescence-quenching assays (Livak et al., PCR Methods Appl. 4:357-362 (1995)), 5′-nuclease allele-specific hybridization TaqMan™ assay (Livak et al., Nature Genet. 9:341-342 (1995)), template-directed dye-terminator incorporation (TDI) assay (Chen and Kwok, Nucl. Acids Res. 25:347-353 (1997)), allele-specific molecular beacon assay (Tyagi et al., Nature Biotech. 16: 49-53 (1998)), PinPoint assay (Haff and Smirnov, Genome Res. 7: 378-388 (1997)), dCAPS analysis (Neffetal., Plant J. 14:387-392 (1998)), pyrosequencing (Ronaghi et al, Analytical Biochemistry 267:65-71 (1999); Ronaghi et al PCT application WO 98/13523; and Nyren et al PCT application WO 98/28440), using mass spectrometry e.g., the Masscode™ system (Howbert et al WO 99/05319; Howber et al WO 97/27331), mass spectroscopy (U.S. Pat. No. 5,965,363, invasive cleavage of oligonucleotide probes (Lyamichev et al Nature Biotechnology 17:292-296), and using high density oligonucleotide arrays (Hacia et al Nature Genetics 22:164-167).

While certain methods for detecting SNPs are described herein, other detection methodologies may be utilized. For example, additional methodologies are known and set forth, in Birren et. al., Genome Analysis, 4:135-186, A Laboratory Manual. Mapping Genomes, Cold Spring Harbor Laboratory Press, Cold Spring Harbor, N.Y. (1999); Maliga et al., Methods in Plant Molecular Biology. A Laboratory Course Manual, Cold Spring Harbor Laboratory Press, Cold Spring Harbor, N.Y. (1995); Paterson, Biotechnology Intelligence Unit: Genome Mapping in Plants, R. G. Landes Co., Georgetown, Tex., and Academic Press, San Diego, Calif. (1996); The Maize Handbook, Freeling and Walbot, eds., Springer-Verlag, New York, N.Y. (1994); Methods in Molecular Medicine: Molecular Diagnosis of Genetic Diseases, Elles, ed., Humana Press, Totowa, N.J. (1996); Clark, ed., Plant Molecular Biology: A Laboratory Manual, Clark, ed., Springer-Verlag, Berlin, Germany (1997).

### Additional Methods of Transformation

Additional methods include, but are not limited to, expression vectors introduced into plant tissues using a direct gene transfer method, such as microprojectile-mediated delivery, DNA injection, electroporation, and the like. More preferably, expression vectors are introduced into plant tissues by using either microprojectile-mediated delivery with a biolistic device or by using Agrobacterium-mediated transformation. Transformant plants obtained with the protoplasm of the subject basil plants of the present application are intended to be within the scope of the embodiments of the application.

### Single-Gene Conversions

When the term basil plant is used in the context of an embodiment of the present application, this also includes any single gene conversions of the subject basil plants of the present application. The term single gene converted plant as used herein refers to those basil plants which are developed by a plant breeding technique called backcrossing wherein essentially all of the desired morphological and physiological characteristics of a variety are recovered in addition to the single gene transferred into the variety via the backcrossing technique. Backcrossing methods can be used with one embodiment of the present application to improve or introduce a characteristic into the variety. The term “backcrossing” as used herein refers to the repeated crossing of a hybrid progeny back to the recurrent parent, i.e., backcrossing 1, 2, 3, 4, 5, 6, 7, 8, or more times to the recurrent parent. The parental basil plant that contributes the gene for the desired characteristic is termed the nonrecurrent or donor parent. This terminology refers to the fact that the nonrecurrent parent is used one time in the backcross protocol and therefore does not recur. The parental basil plant to which the gene or genes from the nonrecurrent parent are transferred is known as the recurrent parent as it is used for several rounds in the backcrossing protocol (Poehlman & Sleper (1994). A backcross conversion of basil occurs when DNA sequences are introduced through backcrossing (Allard, “Principles of Plant Breeding” (1999) with the basil plants of the present application utilized as the recurrent parent. Both naturally occurring and transgenic DNA sequences may be introduced through backcrossing techniques. A backcross conversion may produce a plant with a trait or locus conversion in at least two or more backcrosses, including at least 2 crosses, at least 3 crosses, at least 4 crosses, at least 5 crosses, and the like. Molecular marker assisted breeding or selection may be utilized to reduce the number of backcrosses necessary to achieve the backcross conversion. For example, see, Openshaw, S. J., et al., Marker-assisted Selection in Backcross Breeding, Proceedings Symposium of the Analysis of Molecular Data, Crop Science Society of America, Corvallis, Oreg. (August 1994), where it is demonstrated that a backcross conversion can be made in as few as two backcrosses.

The complexity of the backcross conversion method depends on the type of trait being transferred (single genes or closely linked genes as compared to unlinked genes), the level of expression of the trait, the type of inheritance (cytoplasmic or nuclear), and the types of parents included in the cross. It is understood by those of ordinary skill in the art that for single gene traits that are relatively easy to classify, the backcross method is effective and relatively easy to manage. See, Allard, “Principles of Plant Breeding” (1999). Desired traits that may be transferred through backcross conversion include, but are not limited to, sterility (nuclear and cytoplasmic), fertility restoration, drought tolerance, nitrogen utilization, taste, smell, disease resistance (bacterial, fungal, or viral), insect resistance, and herbicide resistance. In addition, an introgression site itself, such as an FRT site, Lox site, or other site specific integration site, may be inserted by backcrossing and utilized for direct insertion of one or more genes of interest into a specific plant variety. In some embodiments, the number of loci that may be backcrossed into the basil plants of the present application is at least 1, 2, 3, 4, or 5, and/or no more than 6, 5, 4, 3, or 2. A single locus may contain several transgenes, such as a transgene for disease resistance that, in the same expression vector, may also contain a transgene for herbicide resistance. The gene for herbicide resistance may be used as a selectable marker and/or as a phenotypic trait. A single locus conversion of site specific integration system allows for the integration of multiple genes at the converted loci.

The backcross conversion may result from either the transfer of a dominant allele or a recessive allele. Selection of progeny containing the trait of interest is accomplished by direct selection for a trait associated with a dominant allele. Transgenes or genes transferred via backcrossing typically function as a dominant single gene trait and are relatively easy to classify. Selection of progeny for a trait that is transferred via a recessive allele requires growing and selfing the first backcross generation to determine which plants carry the recessive alleles. Recessive traits may require additional progeny testing in successive backcross generations to determine the presence of the locus of interest. The last backcross generation is usually selfed to give pure breeding progeny for the gene(s) being transferred, although a backcross conversion with a stably introgressed trait may also be maintained by further backcrossing to the recurrent parent with selection for the converted trait. As used herein, “crossing” can refer to a simple x by y cross or the process of backcrossing depending on the context.

In addition, the above process and other similar processes described herein may be used to produce first generation progeny basil seed by adding a step at the end of the process that comprises crossing the basil plants of the present application with the introgressed trait or locus with a different basil plant and harvesting the resultant first generation progeny basil seed

Many single gene traits have been identified that are not regularly selected for in the development of a new variety but that can be improved by backcrossing techniques. These traits are well-known in the art.

### Expression Vectors for Basil: Marker Genes

Plant transformation involves the construction of an expression vector which will function in plant cells. Such a vector comprises DNA comprising a gene under control of, or operatively linked to, a regulatory element (for example, a promoter). Expression vectors include at least one genetic marker operably linked to a regulatory element (for example, a promoter) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well-known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin. Another commonly used selectable marker gene is the hygromycin phosphotransferase gene which confers resistance to the antibiotic hygromycin.

Selectable marker genes for plant transformation not of bacterial origin include, for example, mouse dihydrofolate reductase, plant 5-enolpyruvylshikimate-3-phosphate synthase, and plant acetolactate synthase (Eichholtz, et al., Somatic Cell Mol. Genet., 13:67 (1987); Shah, et al., Science, 233:478 (1986); Charest, et al., Plant Cell Rep., 8:643 (1990)).

Another class of marker genes for plant transformation requires screening of presumptively transformed plant cells, rather than direct genetic selection of transformed cells, for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used marker genes for screening presumptively transformed cells include β-glucuronidase (GUS), β-galactosidase, luciferase, and chloramphenicol acetyltransferase (Jefferson, R. A., Plant Mol. Biol. Rep., 5:387 (1987); Teeri, et al., EMBO J., 8:343 (1989); Koncz, et al., Proc. Natl. Acad. Sci. USA, 84:131 (1987); DeBlock, et al., EMBO J., 3:1681 (1984)).

### Expression Vectors for Basil Transformation: Promoters

Genes included in expression vectors must be driven by a nucleotide sequence comprising a regulatory element (for example, a promoter). Several types of promoters are well known in the transformation arts as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters that initiate transcription only in a certain tissue are referred to as “tissue-specific.” A “cell-type” specific promoter primarily drives expression in certain cell types in one or more organs, for example, vascular cells in roots or leaves. An “inducible” promoter is a promoter which is under environmental control. Examples of environmental conditions that may affect transcription by inducible promoters include anaerobic conditions or the presence of light. Tissue-specific, tissue-preferred, cell-type specific, and inducible promoters constitute the class of “non-constitutive” promoters. A “constitutive” promoter is a promoter that is active under most environmental conditions. Many types of promoters are well known in the art.

### Signal Sequences for Targeting Proteins to Subcellular Compartments

Transport of a protein produced by transgenes to a subcellular compartment, such as the chloroplast, vacuole, peroxisome, glyoxysome, cell wall, or mitochondrion, or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine during protein synthesis and processing where the encoded protein is ultimately compartmentalized. Many signal sequences are well-known in the art. See, for example, Becker, et al., Plant Mol. Biol., 20:49 (1992); Knox, C., et al., Plant Mol. Biol., 9:3-17 (1987); Lerner, et al., Plant Physiol., 91:124-129 (1989); Frontes, et al., Plant Cell, 3:483-496 (1991); Matsuoka, et al., Proc. Natl. Acad. Sci., 88:834 (1991); Gould, et al., J. Cell. Biol., 108:1657 (1989); Creissen, et al., Plant 1, 2:129 (1991); Kalderon, et al., Cell, 39:499-509 (1984); Steifel, et al., Plant Cell, 2:785-793 (1990).

### Gene Silencing

Many techniques for gene silencing are well-known to one of skill in the art, including, but not limited to, knock-outs (such as by insertion of a transposable element such as Mu (Vicki Chandler, The Maize Handbook, Ch. 118 (Springer-Verlag 1994)) or other genetic elements such as a FRT, Lox, or other site specific integration sites; antisense technology (see, e.g., Sheehy, et al., PNAS USA, 85:8805-8809 (1988) and U.S. Pat. Nos. 5,107,065, 5,453,566, and 5,759,829); co-suppression (e.g., Taylor, Plant Cell, 9:1245 (1997); Jorgensen, Trends Biotech., 8(12):340-344 (1990); Flavell, PNAS USA, 91:3490-3496 (1994); Finnegan, et al., Bio/Technology, 12:883-888 (1994); Neuhuber, et al., Mol. Gen. Genet., 244:230-241 (1994)); RNA interference (Napoli, et al., Plant Cell, 2:279-289 (1990); U.S. Pat. No. 5,034,323; Sharp, Genes Dev., 13:139-141 (1999); Zamore, et al., Cell, 101:25-33 (2000); Montgomery, et al., PNAS USA, 95:15502-15507 (1998)), virus-induced gene silencing (Burton, et al., Plant Cell, 12:691-705 (2000); Baulcombe, Curr. Op. Plant Bio., 2:109-113 (1999)); target-RNA-specific ribozymes (Haseloff, et al., Nature, 334:585-591 (1988)); hairpin structures (Smith, et al., Nature, 407:319-320 (2000); U.S. Pat. Nos. 6,423,885, 7,138,565, 6,753,139, and 7,713,715); MicroRNA (Aukerman & Sakai, Plant Cell, 15:2730-2741 (2003)); ribozymes (Steinecke, et al., EMBO J., 11:1525 (1992); Perriman, et al., Antisense Res. Dev., 3:253 (1993)); oligonucleotide mediated targeted modification (e.g., U.S. Pat. Nos. 6,528,700 and 6,911,575); Zn-finger targeted molecules (e.g., U.S. Pat. Nos. 7,151,201, 6,453,242, 6,785,613, 7,177,766 and 7,788,044); and other methods or combinations of the above methods known to those of skill in the art.

### Additional Transformation Embodiments

The foregoing methods for transformation may be used for producing a transgenic variety. The transgenic variety could then be crossed with another (non-transformed or transformed) variety in order to produce a new transgenic variety. Alternatively, a genetic trait that has been engineered into a particular basil line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties that do not contain that gene. As used herein, “crossing” can refer to a simplex by y cross or the process of backcrossing depending on the context.

Likewise, by means of one embodiment, commercially important genes can be expressed in transformed plants. More particularly, plants can be genetically engineered to express various phenotypes of commercial interest, including, but not limited to, genes that confer resistance to pests or disease, genes that confer resistance to an herbicide, genes that confer or contribute to a value-added or desired trait, genes that control male sterility, genes that create a site for site specific DNA integration, and genes that affect abiotic stress resistance. Many hundreds if not thousands of different genes are known and could potentially be introduced into a basil plant according to the invention. Non-limiting examples of particular genes and corresponding phenotypes one may choose to introduce into a basil plant include one or more genes for insect tolerance, such as a Bacillus thuringiensis (Bt.) gene, pest tolerance such as genes for fungal disease control, herbicide tolerance such as genes conferring glyphosate tolerance, and genes for quality improvements such as environmental or stress tolerances, or any desirable changes in plant physiology, growth, development, morphology or plant product(s). For example, structural genes would include any gene that confers insect tolerance including but not limited to a Bacillus insect control protein gene as described in WO 99/31248, herein incorporated by reference in its entirety, U.S. Pat. No. 5,689,052, herein incorporated by reference in its entirety, U.S. Pat. Nos. 5,500,365 and 5,880,275, herein incorporated by reference in their entirety. In another embodiment, the structural gene can confer tolerance to the herbicide glyphosate as conferred by genes including, but not limited to Agrobacterium strain CP4 glyphosate resistant EPSPS gene (aroA:CP4) as described in U.S. Pat. No. 5,633,435, herein incorporated by reference in its entirety, or glyphosate oxidoreductase gene (GOX) as described in U.S. Pat. No. 5,463,175, herein incorporated by reference in its entirety. Alternatively, the DNA coding sequences can affect these phenotypes by encoding a non-translatable RNA molecule that causes the targeted inhibition of expression of an endogenous gene, for example via antisense- or cosuppression-mediated mechanisms (see, for example, Bird et al., Biotech. Gen. Engin. Rev., 9:207, 1991). The RNA could also be a catalytic RNA molecule (i.e., a ribozyme) engineered to cleave a desired endogenous mRNA product (see for example, Gibson and Shillito, Mol. Biotech., 7:125, 1997). Thus, any gene which produces a protein or mRNA which expresses a phenotype or morphology change of interest is useful for the practice of one or more embodiments.

### Tissue Culture

Further reproduction of the variety can occur by tissue culture and regeneration. Tissue culture of various tissues of plants and regeneration of plants therefrom is well-known and widely published. For example, reference may be had to do Valla Rego, Luciana et al., Crop Breeding and Applied Technology. 1(3): 283-300 (2001); Komatsuda, T., et al., Crop Sci., 31:333-337 (1991); Stephens, P. A., et al., Theor. Appl. Genet., 82:633-635 (1991); Komatsuda, T., et al., Plant Cell, Tissue and Organ Culture, 28:103-113 (1992); Dhir, S., et al., Plant Cell Reports, 11:285-289 (1992); Pandey, P., et al., Japan J. Breed., 42:1-5 (1992); and Shetty, K., et al., Plant Science, 81:245-251 (1992). Thus, another embodiment is to provide cells which upon growth and differentiation produce basil plants having the physiological and morphological characteristics of the basil plants described in the present application.

Regeneration refers to the development of a plant from tissue culture. The term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as pollen, ovules, embryos, protoplasts, meristematic cells, callus, leaves, anthers, cotyledons, hypocotyl, pistils, roots, root tips, flowers, seeds, petiole, shoot, or stems, and the like. Means for preparing and maintaining plant tissue culture are well-known in the art.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions, and sub-combinations as are within their true spirit and scope.

One or more aspects may be embodied in other specific forms without departing from its spirit or essential characteristics. The described embodiments are to be considered in all respects only as illustrative and not restrictive. The scope of the embodiments is, therefore, indicated by the appended claims rather than by the foregoing description. All changes which come within the meaning and range of equivalency of the claims are to be embraced within their scope.

The foregoing discussion of the embodiments has been presented for purposes of illustration and description. The foregoing is not intended to limit the embodiments to the form or forms disclosed herein. In the foregoing Detailed Description for example, various features of the embodiments are grouped together in one or more embodiments for the purpose of streamlining the disclosure. This method of disclosure is not to be interpreted as reflecting an intention that the claimed embodiments require more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment.

Moreover, though the description of the embodiments has included description of one or more embodiments and certain variations and modifications, other variations and modifications are within the scope of the embodiments (e.g., as may be within the skill and knowledge of those in the art, after understanding the present disclosure). It is intended to obtain rights which include alternative embodiments to the extent permitted, including alternate, interchangeable and/or equivalent structures, functions, ranges or acts to those claimed, whether or not such alternate, interchangeable and/or equivalent structures, functions, ranges or acts are disclosed herein, and without intending to publicly dedicate any patentable subject matter.

The use of the terms “a,” “an,” and “the,” and similar referents in the context of describing the embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice one or more embodiments.

### Deposit Information

A representative sample of proprietary sweet basil seed of the Jacobs Farm Del Cabo and Agroproductos Del Cabo S.A. de C.V., wherein said deposit was designated 165303-6-B and wherein said sweet basil plants grown from said seed express a higher tolerance to Peronospora belbahrii were made with the National Collections of Industrial, Food and Marine Bacteria (NCIMB), Ferguson Building, Craibstone Estate, Bucksburn, Aberdeen, Scotland, AB21 9YA, United Kingdom. The deposit was of at least 2500 seeds and the date of deposit was Feb. 27, 2017. The NCIMB No. is 42726. Upon issuance of a patent, all restrictions on the availability to the public of the deposit will be irrevocably removed consistent with all of the requirements of 37 C.F.R. §§ 1.801-1.809. The deposit will be maintained in the depository for a period of thirty years, or five years after the last request, or for the enforceable life of the patent, whichever is longer, and will be replaced as necessary during the period.

What is claimed is:

1. An Ocimum basilicum plant or germplasm that exhibits tolerance or improved tolerance to Peronospora belbahrii infection, comprising in its genome the single nucleotide polymorphisms (SNPs) of SEQ ID NOs:1-630.

2. The Ocimum basilicum plant or germplasm of claim 1, wherein said plant or germplasm comprises approximately 90% of any combination of the single nucleotide polymorphisms (SNPs) of SEQ ID NOs:1-630.

3. The Ocimum basilicum plant or germplasm of claim 1, wherein said plant or germplasm comprises approximately 80% of any combination of the single nucleotide polymorphisms (SNPs) of SEQ ID NOs:1-630.

4. The Ocimum basilicum plant or germplasm of claim 1, wherein said plant or germplasm comprises approximately 70% of any combination of any combination of the single nucleotide polymorphisms (SNPs) of SEQ ID NOs:1-630.

5. The Ocimum basilicum plant or germplasm of claim 1, wherein said plant or germplasm comprises approximately 60% of any combination of the single nucleotide polymorphisms (SNPs) of SEQ ID NOs:1-630.

6. The Ocimum basilicum plant or germplasm of claim 1, wherein said plant or germplasm comprises approximately 50% of any combination of the single nucleotide polymorphisms (SNPs) of SEQ ID NOs:1-630.

7. The Ocimum basilicum plant or germplasm of claim 1, wherein said plant or germplasm comprises approximately 40% of any combination of the single nucleotide polymorphisms (SNPs) of SEQ ID NOs:1-630.

8. The Ocimum basilicum plant or germplasm of claim 1, wherein said plant or germplasm comprises approximately 30% of any combination of the single nucleotide polymorphisms (SNPs) of SEQ ID NOs:1-630.

9. The Ocimum basilicum plant or germplasm of claim 1, wherein said plant or germplasm comprises approximately 20% of any combination of the single nucleotide polymorphisms (SNPs) of SEQ ID NOs:1-630.

10. The Ocimum basilicum plant or germplasm of claim 1, wherein said plant or germplasm comprises approximately 10% of any combination of the single nucleotide polymorphisms (SNPs) of SEQ ID NOs:1-630.

11. The Ocimum basilicum plant or germplasm of claim 1, wherein said plant or germplasm comprises approximately 5% of any combination of the single nucleotide polymorphisms (SNPs) of SEQ ID NOs:1-630.

12. The Ocimum basilicum plant or germplasm of claim 1, wherein said plant or germplasm comprises 0.5% of any combination of the single nucleotide polymorphisms (SNPs) of SEQ ID NOs:1-630.

13. A method of producing an Ocimum basilicum plant or germplasm that exhibits tolerance or improved tolerance to Peronospora belbahrii infection, the method comprising: isolating nucleic acids from a genome of a first Ocimum basilicum plant or germplasm; detecting in the first Ocimum basilicum plant or germplasm at least one or more single nucleotide polymorphisms (SNPs) of SEQ ID NOs:1-630 that is associated with the tolerance or improved tolerance to Peronospora belbahrii infection; selecting said first Ocimum basilicum plant or germplasm, or selecting a progeny of said first Ocimum basilicum plant or germplasm wherein the plant, germplasm or progeny comprises at least one or more single nucleotide polymorphisms (SNPs) of SEQ ID NOs:1-630 associated with tolerance or improved tolerance to Peronospora belbahrii infection; and introgressing the at least one or more single nucleotide polymorphisms (SNPs) of SEQ ID NOs:1-630 associated with tolerance or improved tolerance to Peronospora belbahrii infection of the first Ocimum basilicum plant or germplasm into a second Ocimum basilicum plant or germplasm to produce an introgressed Ocimum basilicum plant or germplasm.

14. An Ocimum basilicum plant or germplasm that exhibits tolerance or improved tolerance to Peronospora belbahrii produced by the method of claim 13.

15. A method of selecting an Ocimum basilicum plant or germplasm that exhibits tolerance or improved tolerance to Peronospora belbahrii infection, the method comprising: detecting in an Ocimum basilicum plant one or more loci associated with a polynucleotide, wherein the polynucleotide discriminates between an Ocimum basilicum that exhibits tolerance or improved tolerance and an Ocimum basilicum that exhibits susceptibility to Peronospora belbahrii infection, and wherein the polynucleotide detects at least one or more SNP within a genomic sequence set forth in SEQ ID NOs:1-630; and selecting the Ocimum basilicum plant or germplasm comprising the detected one or more loci, thereby selecting an Ocimum basilicum plant with tolerance or improved tolerance to Peronospora belbahrii infection.

16. A sweet basil plant, wherein said plant comprises at least one dominant allele and at least a recessive allele in the homozygous state, wherein when both said alleles are present together in said states in the genome of said plant, are responsible for higher levels of tolerance or improved tolerance to Peronospora belbahrii, and wherein a sample of representative seed of said sweet basil plants comprised of said genes responsible for higher levels of tolerance or improved tolerance to Peronospora belbahrii has been deposited under NCIMB No. 42726.

17. The sweet basil plant of claim 16, wherein said sweet basil plant is a tetraploid.

18. The sweet basil plant of claim 16, wherein said sweet basil plant is a diploid.

19. A sweet basil seed produced by growing the plant of claim 16.

20. A sweet basil plant, or a plant part thereof, produced by growing the seed of claim 19.

21. The plant part of claim 20, wherein the plant part comprises a cell, seed, protoplast, tissue culture, or vegetative cutting.

22. A tissue culture produced from protoplasts or cells from the plant of claim 16, wherein said cells or protoplasts are produced from a plant part selected from the group consisting of pollen, ovules, embryos, protoplasts, meristematic cells, callus, leaves, anthers, cotyledons, hypocotyl, pistils, roots, root tips, flowers, seeds, petiole, and stems.

23. A sweet basil plant regenerated from the tissue culture of claim 22.

24. A method of vegetatively propagating the plant of claim 16, comprising the steps of:
collecting tissue or cells capable of being propagated from a plant according to claim 16;
cultivating said tissue or cells to obtain proliferated shoots; and
rooting said proliferated shoots to obtain rooted plantlets; or
cultivating said tissue or cells to obtain proliferated shoots, or to obtain plantlets.

25. A sweet basil plant produced by growing the plantlets or proliferated shoots of claim 24.

26. A method for producing basil seed, said method comprising crossing two basil plants and harvesting the resultant sweet basil seed, wherein at least one basil plant is the basil plant of claim 16.

27. A method of determining the genotype of the sweet basil plant of claim 16, wherein said method comprises obtaining a sample of nucleic acids from said plant and detecting in said nucleic acids a plurality of polymorphisms.

28. A method for developing a sweet basil plant in a basil plant breeding program, comprising applying plant breeding techniques comprising recurrent selection, backcrossing, pedigree breeding, marker enhanced selection, haploid/double haploid production, or transformation to the sweet basil plant of claim 16, or its parts, wherein application of said techniques results in development of a new basil plant.

29. A method of inducing a mutation into the genome of the sweet basil plant of claim 16, said method comprising inducing a mutation to the plant, or plant part thereof of said plant and wherein said mutation is selected from the group consisting of ionizing radiation, chemical mutagens, targeting induced local lesions in genomes, zinc finger nuclease mediated mutagenesis, meganucleases, and gene editing, and wherein the resulting plant comprises at least one genome mutation.

30. A mutagenized plant produced by the method of claim 29.

31. A method of evaluating the response of an O. basilicum plant to Peronospora belbahrii, comprising the steps of:
sowing O. basilicum seeds into one or more plug trays and placed in conditions to promote germination, wherein said seeds are comprised of one or more lines for testing, a susceptible control, and a resistant control;
growing said plants in said plug trays to the stage where each plant has developed two true leaves, and inoculating said plants with Peronospora belbahrii inoculum solution, wherein the inoculum solution consists of fresh harvested leaves where sporulation has recently taken place that are placed in a container with water and agitated, and wherein the inoculum solution is adjusted to a concentration of 50,000 spores per milliliter of water using a hemocytometer, and wherein the inoculum solution is applied to the upper surface of the leaves using a hand sprayer;
transferring the inoculated plants in the plug trays to a closed plastic chamber for 16 hours, wherein the conditions in the chamber are total darkness, a temperature range of 19 degrees Celsius to 21 degrees Celsius, a relative humidity range between 85% to 90% for the first 4 hours, and for hours 4 through 16, the relative humidity is greater than 90%;
recording the relative humidity inside the plastic chamber;
removing the plants in the plug trays from the plastic chamber and transferring said plants to a different location with full sunlight under natural day length and night length for 6 days;
transferring the plants in the plug trays back into a plastic chamber for about 16 hours, wherein the conditions in the chamber are total darkness, a temperature range of 19 degrees Celsius to 21 degrees Celsius, a relative humidity range between 85% to 90% for the first 4 hours, and for hours 4 through 16, the relative humidity is greater than 90%;
removing said plants and plug trays from the plastic chamber;
observing and rating the degree of infection of Peronospora belbahrii disease development by evaluating the level of sporulation on the lower surface of each leaf of the plant.


SEQUENCE LISTING (630 sequences)

SEQ ID NO: 1 - DNA, 121 bp, Ocimum basilicum
  variation (61)..(61): at position 61, “n” is either A or G
tttataatataaaatcttctctgtgagcttatgaattttgcttgtatctaacttgatcttntatctatcagtttatgatgagagagccagaatcatgtaaattagcctgccgagtggaact

SEQ ID NO: 2 - DNA, 121 bp, Ocimum basilicum
  variation (61)..(61): at position 61, “n” is either G or A
gggtataccagtactctctaaagtatccatgtatccatcacgaacatctaatgtttcgtanccgtgagatcaaatacattctctaagaatgtgatgcagaccacatgccaacctatcacat

SEQ ID NO: 3 - DNA, 121 bp, Ocimum basilicum
  variation (61)..(61): at position 61, “n” is either G or C
cccatacaaatctatagctttttcaagactctgttcactcaaactaagccgattcactttntcattcccgctcatcatcagcgcttgcctcaacccttttcgggcattacccgaaagaatg

SEQ ID NO: 4 - DNA, 121 bp, Ocimum basilicum; identical to SEQ ID NO: 3
  variation (61)..(61): at position 61, “n” is either G or C

SEQ ID NO: 5 - DNA, 121 bp, Ocimum basilicum
  variation (61)..(61): at position 61, “n” is either T or C
gcgatttgacatgatatatgtggactcagatcaagagagaaagcgaccaatcatgatacanagagcagttcttggatcattggagcggttcttcggggttcttatagaacattatgctgga

SEQ ID NO: 6 - DNA, 121 bp, Ocimum basilicum
  variation (61)..(61): at position 61, “n” is either C or T
tcgaaggtgctctctctctctcgtgtgatttctttacacttgcattgcatactgagattgntctcttgcaggaattggctacactctagaccccgatttttcctttgcaacaattgctgca

SEQ ID NO: 7 - DNA, 121 bp, Ocimum basilicum
  variation (61)..(61): at position 61, “n” is either A or T
gacaagcatgtattgcaagggtaggctatcaacttatactactatataccactgtataaangaaaaacctaaagaaaattaaatttataatgatgcaacaaaggacttgaaagataaataa

SEQ ID NO: 8 - DNA, 121 bp, Ocimum basilicum
  variation (61)..(61): at position 61, “n” is either be an A or G
gcagggacttgatccaaaatgtaaacaacctgaaaatatattcaagaaaaagacgatttgnataacgaaaaataatccatctgcatatatataccaacttgtgtattgtcatacctggcct

SEQ ID NO: 9 - DNA, 121 bp, Ocimum basilicum
  variation (61)..(61): at position 61, “n” is either be a T or C
agcgttatcatttatggatggatcatctggatacaatcaaatgcgcatggcgccagaggangaagaaataacggtgtttcgcacaccgaagggaatatattgctacaaagtgatgccattt

SEQ ID NO: 10 - DNA, 121 bp, Ocimum basilicum
  variation (61)..(61): at position 61, “n” is either T or C
ctgtctggcagcccacctactcaaaatcctagagtgacgcctcctggcggcagaagattcngccaggtcatcaccatcgagatctgacctccttctccttctgatgagttgatccccttca

SEQ ID NO: 11 - DNA, 121 bp, Ocimum basilicum
  variation (61)..(61): at position 61, “n” can be either A or G
gggaagaagctggtgtatttggacaacgccgccacgtcgcagaagccgattgctgtgctgnacgctctgaagagttattatgaaggatataattcgaacgttcatcgtgggattcattatt

SEQ ID NO: 12 - DNA, 121 bp, Ocimum basilicum
  variation (61)..(61): at position 61, “n” is either A or T
tcaaaatggttaacccaagttgttagagagtccattaaacccaccttataaaccatagaanattcatgtacccaactaatgtggtatattacatacttcccccgtttagagacacaacgtc

SEQ ID NO: 13 - DNA, 121 bp, Ocimum basilicum
  variation (61)..(61): at position 61, “n” is either G or C
tattgtctacttttatcaattactatagtctttatttgatatccctaaccaatgttttgantttgcaggaaaatgacttctttattccggacgacattttccggcatcatgataagagagc

SEQ ID NO: 14 - DNA, 121 bp, Ocimum basilicum
  variation (61)..(61): at position 61, “n” is either A or T
atgccgggttcaatttgaccgtcatgagctgggggttgaatttgtcatggtgggatatttntgctctttaagcttttgatattgtcatgtataaattttgatattgcattattcagatatg

SEQ ID NO: 15 - DNA, 121 bp, Ocimum basilicum
  variation (61)..(61): at position 61, “n” is either A or G
agaggtgcatctcggctcattcatgagttcccaagccataattgtgggatcattcttgtanaggagtccagtaattgtattatatctgttcaggacagtctgcaaacacatatactcacac

SEQ ID NO: 16 - DNA, 121 bp, Ocimum basilicum
  variation (61)..(61): at position 61, “n” is either G or A
agccataattgtgggatcattcttgtaaaggagtccagtaattgtattatatctgttcagnacagtctgcaaacacatatactcacaccaaattataaataatgaagcagataatcttgat

SEQ ID NO: 17 - DNA, 121 bp, Ocimum basilicum
  variation (61)..(61): at position 61, “n” is either A or T
ggtttttcttttctttccttttcttttgtttttgaaagtgggcacatatataatatttttnattggagcacacgtggatgtatatcctcatttgattaattagttagctgctcgatggttt

SEQ ID NO: 18 - DNA, 121 bp, Ocimum basilicum
  variation (61)..(61): at position 61, “n” is either T or C
agttgaggtcggacaggttttccaacttcaatgtcctgcagaagagtagctagcatcatgntcattcactgaacatatgttgaagatgaaaggactaaatattccaaattaaaatttgaga

SEQ ID NO: 19 - DNA, 121 bp, Ocimum basilicum
  variation (61)..(61): at position 61, “n” is either C or T
tcggacaggttttccaacttcaatgtcctgcagaagagtagctagcatcatgttcattcantgaacatatgttgaagatgaaaggactaaatattccaaattaaaatttgagaagaacaga

SEQ ID NO: 20 - DNA, 121 bp, Ocimum basilicum
  variation (61)..(61): at position 61, “n” is either A or G
aatttcttttcacttgcaaagatatgctgcgcaactgaaatgaatttccgtggatagatcnccggtgttgatggaggaattgtttcagtacatgaggacgttccgttctcagatcaatggt

SEQ ID NO: 21 - DNA, 121 bp, Ocimum basilicum
  variation (61)..(61): at position 61, “n” is either T or C
ctgtaaaacattctcagcttcccaggttttattagaagttccaattgatgaggtgagaagnaagtcagtgtcgcttaaatatgttccaacgacccatgcatcctcgattgcctcctcgctc

SEQ ID NO: 22 - DNA, 121 bp, Ocimum basilicum
  variation (61)..(61): at position 61, “n” is either A or G
gaatacatttttggaaaacaaaactcaacaaccatcatcttgcacctcggatctaatatgncagctatggacatcaacaaattacactcaccccaatacttatcaaatttttacttcatag

SEQ ID NO: 23 - DNA, 121 bp, Ocimum basilicum
  variation (61)..(61): at position 61, “n” is either A or G
tatagttgttcaatctcttacgagtgcagtttcaagagggcggattgctccggtttatctnttccaaggtgctcgtggaattggaaaaacttcagctgctagaatttttggtgctgctttg

SEQ ID NO: 24 - DNA, 121 bp, Ocimum basilicum
  variation (61)..(61): at position 61, “n” is either C or T
ggctgcatcacgaattcttgcttcggactccattttagcttcctcaaacttggaatccaanctcgagcataaaagctcctgttctgacaatttaggcaagcccacttgcttgagaaatgcc

SEQ ID NO: 25 - DNA, 121 bp, Ocimum basilicum
  variation (61)..(61): at position 61, “n” is either G or C
gatgatcccctttggaggagggcggaggggatgcccaggaggggtgatgggtttggtgtcngcagagctggctttggccaatcttctctactcatttgactgggtgttgccctccctcact

SEQ ID NO: 26 - DNA, 121 bp, Ocimum basilicum
  variation (61)..(61): at position 61, “n” is either C or G
gcagtagctgggagaatatgttgccattggtagaattcgcatacaataacagttatcaagnaaccattggaatggcaccatatgaagcattgtatgggaagagaggcagatctccgctcta

SEQ ID NO: 27 - DNA, 121 bp, Ocimum basilicum
  variation (61)..(61): at position 61, “n” is either A or G
ggagaatatgttgccattggtagaattcgcatacaataacagttatcaagcaaccattggnatggcaccatatgaagcattgtatgggaagagaggcagatctccgctctattgggatgaa

SEQ ID NO: 28 - DNA, 121 bp, Ocimum basilicum
  variation (61)..(61): at position 61, “n” is either A or G
tatgttgccattggtagaattcgcatacaataacagttatcaagcaaccattggaatggcnccatatgaagcattgtatgggaagagaggcagatctccgctctattgggatgaagtaggc

SEQ ID NO: 29 - DNA, 121 bp, Ocimum basilicum
  variation (61)..(61): at position 61, “n” is either A or G
ggtagaattcgcatacaataacagttatcaagcaaccattggaatggcaccatatgaagcnttgtatgggaagagaggcagatctccgctctattgggatgaagtaggcgaaaggaaaata

SEQ ID NO: 30 - DNA, 121 bp, Ocimum basilicum
  variation (61)..(61): at position 61, “n” is either T or C
attcgcatacaataacagttatcaagcaaccattggaatggcaccatatgaagcattgtangggaagagaggcagatctccgctctattgggatgaagtaggcgaaaggaaaatacttaga

SEQ ID NO: 31 - DNA, 121 bp, Ocimum basilicum
  variation (61)..(61): at position 61, “n” is either A or C
aacagttatcaagcaaccattggaatggcaccatatgaagcattgtatgggaagagaggcngatctccgctctattgggatgaagtaggcgaaaggaaaatacttagacctaatgcaatta

SEQ ID NO: 32 - DNA, 121 bp, Ocimum basilicum
  variation (61)..(61): at position 61, “n” is either G or T
tcaagcaaccattggaatggcaccatatgaagcattgtatgggaagagaggcagatctccnctctattgggatgaagtaggcgaaaggaaaatacttagacctaatgcaattaatgagatg

SEQ ID NO: 33 - DNA, 121 bp, Ocimum basilicum
  variation (61)..(61): at position 61, “n” is either G or A
tttaaaagtaaagaatatgttgtaaaattcagcatcttagtttgaattttagttggggagncactttcttcaattcaaatgaggtttaacaggctaattgaaagttgggatatgacatctg

SEQ ID NO: 34 - DNA, 121 bp, Ocimum basilicum
  variation (61)..(61): at position 61, “n” is either T or C
atgtcacaaacttacttcttaatttaaaataaaagtaatggaatggatcactttcaatccntagcaaccatagttggatcaacagtgatttagggtgtgacatgtcatttcacattgacaa

SEQ ID NO: 35 - DNA, 121 bp, Ocimum basilicum
  variation (61)..(61): at position 61, “n” is either G or A
taagacaaatgactcatatttattatacctacgcttatgatttatggaaatcaatttatantcagaatgaacaaatgttcccaatgctgatgagtcgaaccacccacatttgaacaatgtc

SEQ ID NO: 36 - DNA, 121 bp, Ocimum basilicum
  variation (61)..(61): at position 61, “n” is either C or A
cttctgacatgctaatctcctcgaggcccttgcataaattggtctccagaatggaacatcnaaatgtggagtgtctggatatttgaaggccaattcccctctcattgccctactccctcag

SEQ ID NO: 37 - DNA, 121 bp, Ocimum basilicum
  variation (61)..(61): at position 61, “n” is either T or C
aagagttctcatcaatttccaaacttacaatgaactatcaaggatatctaattcgaatgtntggggccagagataaaccttcgcaacttgtcttgccacaacttcctgccaaaaaccaaag

SEQ ID NO: 38 - DNA, 121 bp, Ocimum basilicum
  variation (61)..(61): at position 61, “n” is either A or G
aactctttgcgggcaggcgtatggggcgaagcagtatcacatgatgggaatctacctgcancgatcatggatcattgatctcatcacgctgcttgttttgcttcctgtgttcatctttggg

SEQ ID NO: 39 - DNA, 121 bp, Ocimum basilicum
  variation (61)..(61): at position 61, “n” is either C or T
gctccagttccactcgatcctcgtcataaacaggagcggcctcctgcaagtgacggacctntggtacgactggcccaacggccgcaatttcaacatcatccaacatcagctcggtaaaaag

SEQ ID NO: 40 - DNA, 121 bp, Ocimum basilicum
  variation (61)..(61): at position 61, “n” is either C or T
tatcggattcaacagatgcaatgccgatcactgctgctacttcaagaggtttgagggtggntatattattctattgctctacattgatgacatgttgcttgctccagtgctaggtaaatag

SEQ ID NO: 41 - DNA, 121 bp, Ocimum basilicum
  variation (61)..(61): at position 61, “n” is either A or T
agaaggaacttgtgtgaacaatcactacactgtgatgttcaagaaggtttttacttattcncatgccattagatataaattaattttgctttctgctaatcttaaatccattatctctttc

SEQ ID NO: 42 - DNA, 121 bp, Ocimum basilicum
  variation (61)..(61): at position 61, “n” is either T or C
acttgaagttcgaaagattataaacttgcaatatttagcaagtaacttgtcggatgcattnaatgatcataaaggagtgactaaatcacatatacctgctataaatgcacaggaaagaatt

SEQ ID NO: 43 - DNA, 121 bp, Ocimum basilicum
  variation (61)..(61): at position 61, “n” is either A or T
cttcaagagtgtgtgaacttgcaagtgtgctctagtttggccagaatctttctccagtatnttatttatttgagccttagctatttgatgggcaacaggatcgacgtcgaacccaacaaag

SEQ ID NO: 44 - DNA, 121 bp, Ocimum basilicum
  variation (61)..(61): at position 61, “n” is either T or C
agagtgtgtgaacttgcaagtgtgctctagtttggccagaatctttctccagtatattatntatttgagccttagctatttgatgggcaacaggatcgacgtcgaacccaacaaagaactc

SEQ ID NO: 45 - DNA, 121 bp, Ocimum basilicum
  variation (61)..(61): at position 61, “n” is either T or G
tgtgtgaacttgcaagtgtgctctagtttggccagaatctttctccagtatattatttatntgagccttagctatttgatgggcaacaggatcgacgtcgaacccaacaaagaactccatt

SEQ ID NO: 46 - DNA, 121 bp, Ocimum basilicum
  variation (61)..(61): at position 61, “n” is either G or C
tctagtttggccagaatctttctccagtatattatttatttgagccttagctatttgatgngcaacaggatcgacgtcgaacccaacaaagaactccatttcaggatgagcctgaataatc

SEQ ID NO: 47 - DNA, 121 bp, Ocimum basilicum
  variation (61)..(61): at position 61, “n” is either G or T
ttctacttttctttctttttggtgggtttatgttgagttttgagcaacctagtgttttgantgcagccgcgtcagaaggcaggattcatgcaagggtctcagttccatccaggtaattctc

SEQ ID NO: 48 - DNA, 121 bp, Ocimum basilicum
  variation (61)..(61): at position 61, “n” is either A or T
tcttaagaagatggaattgaaattaaccatagttaagaagaatctataaaggaaatacaanaaatgttcgtaataacactaacaaaattctaaattttgacagcgtaatgttctattccaa

SEQ ID NO: 49 - DNA, 121 bp, Ocimum basilicum
  variation (61)..(61): at position 61, “n” is either A or C
atatatagtagcggataagcgagcagcaacaattattttatcactccaacggatagctttntccctacgtttgattatacggtttttgttctttcaagagacgtaattacccttacttaca

SEQ ID NO: 50 - DNA, 121 bp, Ocimum basilicum
  variation (61)..(61): at position 61, “n” is either G or A
agcagcaacaattattttatcactccaacggatagctttatccctacgtttgattatacgntttttgttctttcaagagacgtaattacccttacttacaagctaaacaaatttacaatta

SEQ ID NO: 51 - DNA, 121 bp, Ocimum basilicum
  variation (61)..(61): at position 61, “n” is either G or A
cttacataggtgatctcgtattcaaagtattttgctataattcacctttacaaggttcagnaatcattaagaatcatatgattcagccttttaccacatgcaggtcttgacacttttttgc

SEQ ID NO: 52 - DNA, 121 bp, Ocimum basilicum
  variation (61)..(61): at position 61, “n” is either T or A
tattcaaagtattttgctataattcacctttacaaggttcaggaatcattaagaatcatangattcagccttttaccacatgcaggtcttgacacttttttgcttttttagggaagggtgc

SEQ ID NO: 53 - DNA, 121 bp, Ocimum basilicum
  variation (61)..(61): at position 61, “n” is either A or T
ttcaaagtattttgctataattcacctttacaaggttcaggaatcattaagaatcatatgnttcagccttttaccacatgcaggtcttgacacttttttgcttttttagggaagggtgcaa

SEQ ID NO: 54 - DNA, 121 bp, Ocimum basilicum
  variation (61)..(61): at position 61, “n” is either A or G
ttcaatttgatgaaaaccgacttcaccttattattttctagagaattgtgaaaccaaaatnttatatctacttcatttaatgagtccagcagttcctgagttgcttccctccactaaatcc

SEQ ID NO: 55 - DNA, 121 bp, Ocimum basilicum
  variation (61)..(61): at position 61, “n” is either C or T
actgagaagaatgagtgtggccctgatgtgaattagagttcttgttcacatcaataactanatggccatcgccatttgtttgatgagaatggcttacaccattcgtgacgtttgcacttcg

SEQ ID NO: 56 - DNA, 121 bp, Ocimum basilicum
  variation (61)..(61): at position 61, “n” is either T or A
tcatgttttacgatttacctgcagactcaaaaatgaccgtggtaggattgctggagcgatnagagcttgtacctcttcattaatttttatgttctgaacagctatctgtcagagagcaaac

SEQ ID NO: 57 - DNA, 121 bp, Ocimum basilicum
  variation (61)..(61): at position 61, “n” is either G or A
gagcactgtcttggtgagattgcatttttctgcaatgatgttaagcactttcatggcatcngagtgtggcagataatacaggattccctccaaaatccagactgtgctcttgttaggctcg

SEQ ID NO: 58 - DNA, 121 bp, Ocimum basilicum
  variation (61)..(61): at position 61, “n” is either A or G
ttttgtgactgtccatttaccggaactcacttttctaactaaaatcatggccctgcaaccnaccctagtttcaaccctttgcctgactattttttcacgtttgtcaggcattctgaatccc

SEQ ID NO: 59 - DNA, 121 bp, Ocimum basilicum
  variation (61)..(61): at position 61, “n” is either C or G
ttgtgattttcgagctcttcctgaaccgggtttggacgacggccaattgacacccgactcngttttagaaatgtccggttcagactcggccggtttgggtcagtgctctagtcttctcctc

SEQ ID NO: 60 - DNA, 121 bp, Ocimum basilicum
  variation (61)..(61): at position 61, “n” is either G or C
ttcctgaaccgggtttggacgacggccaattgacacccgactccgttttagaaatgtccgnttcagactcggccggtttgggtcagtgctctagtcttctcctcaacgccactgcaaaaat

SEQ ID NO: 61 - DNA, 121 bp, Ocimum basilicum
  variation (61)..(61): at position 61, “n” is either T or C
tgtaagcacgaaacattcttcattatcctgttatcgtttctgcttttcaggattttctgcnttgctgtattggctgtacttatagcattagtagcttctagcccaactaaaggatggagtg

SEQ ID NO: 62 - DNA, 121 bp, Ocimum basilicum
  variation (61)..(61): at position 61, “n” is either C or T
cctacgtcttgtggttcttggcttattttttcactataggattctccatccagttcctgangcatatggcttgtggatgacatcaattatctgtgaaatatggtttggtgtatcatggtta

SEQ ID NO: 63 - DNA, 121 bp, Ocimum basilicum
  variation (61)..(61): at position 61, “n” is either a G or A
ataattatgagttataatacaaaatctgtaaaagtataatgcatgtatttgtgtgataatnaaatgcattaatgaaggtaaaaatctacattatatcacatagattacgcattataactat

SEQ ID NO: 64 - DNA, 121 bp, Ocimum basilicum
  variation (61)..(61): at position 61, “n” is either G or C
aggacgagtctttattttcatcctctcgaatcgtccctccatttgaagcaagttcttttgncactttagtcttcttctttggataaatatgtactgaaggaggtaagagggtctcttgaat

SEQ ID NO: 65 - DNA, 121 bp, Ocimum basilicum
  variation (61)..(61): at position 61, “n” is either T or A
aaaacaaaagcatcagaaaattcatacaaatggaagctcaacagtagaaacactcacatantcataaatgagcatacaaaattctgctgaaaaagaacaatcaaattattacaactcaaat

SEQ ID NO: 66 - DNA, 121 bp, Ocimum basilicum
  variation (61)..(61): at position 61, “n” is either G or A
catcgtgaaacaaattctaccattgttgtataacgtcgtcaattcttgtatctatgctttnaatgttagtaaatctgaacccatacagagttgggggttttcggccttgatagattgtctg

SEQ ID NO: 67 - DNA, 121 bp, Ocimum basilicum
  variation (61)..(61): at position 61, “n” is either C or T
acgatgcgctgagatgctcccgtccgaggcattgatcacgatgtcggtgtggatactttcngataacattcgaccgagcgatgtgagagctgtttcatgcaacagtgtttgcggaaaccct

SEQ ID NO: 68 - DNA, 121 bp, Ocimum basilicum
  variation (61)..(61): at position 61, “n” is either G or T
tctcttaactgcatcaatagctaaatttgctacttctgcccccttgatttcagccgagttnccgtatgaccagaaaaggaagattcctgtgtctgagtaaaacctctgcattgctacaggg

SEQ ID NO: 69 - DNA, 121 bp, Ocimum basilicum
  variation (61)..(61): at position 61, “n” is either G or A
ctcctccacaacgtagatcgagttaacaatattgaagcaataaaacttcaactttcatctnttctcacgtttgtttccatttttgccctggaaaatccctttggattcataagtatttcca

SEQ ID NO: 70 - DNA, 121 bp, Ocimum basilicum
  variation (61)..(61): at position 61, “n” is either G or A
agtaattgtcaaagataccctccatttgagggtcaataggttcgccagtgtgaagatttantccaaccacattacccttgtcatgttctccctgaaaagagcaaatcaatgatgaactgcg

SEQ ID NO: 71 - DNA, 121 bp, Ocimum basilicum
  variation (61)..(61): at position 61, “n” is either A or G
catgaacaacaaaaacacaaacagatccgtatggatgggaatcaagaaaaggagacccatntgcaaaaaaggcggtaaaaatgcaacctttctgtgggaatccatggcgtttgaaagtggg

SEQ ID NO: 72 - DNA, 121 bp, Ocimum basilicum
  variation (61)..(61): at position 61, “n” is either A or C
cacccaagtctggagttatctgtttcgacgttttagaagacgtgttagcaaccacagctgnatttattttactaagagatgacatcaatacttgagaggacctccatccatgcaccccagg

SEQ ID NO: 73 - DNA, 121 bp, Ocimum basilicum
  variation (61)..(61): at position 61, “n” is either A or G
ttaaatttattaaatctgaaaatataagcaaattgatatttaaatatttaaattcacatgncgtgatttgattcaccgtgaatattccagtgtctacagggtccactatgaataaaaactt

SEQ ID NO: 74 - DNA, 121 bp, Ocimum basilicum
  variation (61)..(61): at position 61, “n” is either A or T
acacctcttgtattccttgaatagcttatgtattacacaacttgtagagagagtatgttgngatggtcctacagttctctcctgagttgtgaacatacattctcaccttcttcccatcaaa

SEQ ID NO: 75 - DNA, 121 bp, Ocimum basilicum
  variation (61)..(61): at position 61, “n” is either T or A
cttgtattccttgaatagcttatgtattacacaacttgtagagagagtatgttgagatggncctacagttctctcctgagttgtgaacatacattctcaccttcttcccatcaaacttggc

SEQ ID NO: 76 - DNA, 121 bp, Ocimum basilicum
  variation (61)..(61): at position 61, “n” is either C or A
ttgaatagcttatgtattacacaacttgtagagagagtatgttgagatggtcctacagttntctcctgagttgtgaacatacattctcaccttcttcccatcaaacttggcagtaactgat

SEQ ID NO: 77 - DNA, 121 bp, Ocimum basilicum
  variation (61)..(61): at position 61, “n” is either A or G
attacacaacttgtagagagagtatgttgagatggtcctacagttctctcctgagttgtgnacatacattctcaccttcttcccatcaaacttggcagtaactgatctcttcactaaatca

SEQ ID NO: 78 - DNA, 121 bp, Ocimum basilicum
  variation (61)..(61): at position 61, “n” is either C or G
agagagagtatgttgagatggtcctacagttctctcctgagttgtgaacatacattctcancttcttcccatcaaacttggcagtaactgatctcttcactaaatcaaacttcaccgggtt

SEQ ID NO: 79 - DNA, 121 bp, Ocimum basilicum
  variation (61)..(61): at position 61, “n” is either T or C
cagattttcaaagttttgatccaaaagcaagataactaggaacctacagaagaaaaacctnagcaataagaagttcaaccaaactgataaagaatcaaaccacttcttttatccatgtaat

SEQ ID NO: 80 - DNA, 121 bp, Ocimum basilicum
  variation (61)..(61): at position 61, “n” is either T or G
ttaaaaccatattccgacatcacaacagcaggttccccttttccagtggcaacgatgaatncattttcatacctacaaaggaaggcaacaaaagaaatgaaagttcattagtaattagtac

SEQ ID NO: 81 - DNA, 121 bp, Ocimum basilicum
  variation (61)..(61): at position 61, “n” is either C or T
acccaaaagttcggaaaattcttgattatcgaacctccatacttgacggtgttaataagcnatccaacgaaagatgatgtgatcattatataatgtgaagaaacgactgcaattgaattaa

SEQ ID NO: 82 - DNA, 121 bp, Ocimum basilicum
  variation (61)..(61): at position 61, “n” is either T or G
cattaccggcatgaatggcgatgagacgtttatattccggtgataccaaattgcccgattntttaccgcaggtgtcaaactcttcataatcactgtcactctgagaaaatagtatattaaa

SEQ ID NO: 83 - DNA, 121 bp, Ocimum basilicum
  variation (61)..(61): at position 61, “n” is either A or T
agttcctcgtgttgaacacaataaactccgcctagtttgcaaaaggtggttcaagctcctngctggaaatttcttctactctctcaggaagagtctcggtatggcagaagaatgggtatac

SEQ ID NO: 84 - DNA, 121 bp, Ocimum basilicum
  variation (61)..(61): at position 61, “n” is either C or T
gacatcgcttccgagctcggaattgagagagagaagcatttgggtgagttgagggttgaanagtccggcagctctttggagctcagcagcgcactcgccgtttcggctgtgctgagccaat

SEQ ID NO: 85 - DNA, 121 bp, Ocimum basilicum
  variation (61)..(61): at position 61, “n” is either T or G
tcaatcagaagttacgctgtttcaacttttgtcttttagttgatgagaattaatacagtgnctttttataaattattgcagaattatatgtgttttgttcgataaggttaaagaagccagc

SEQ ID NO: 86 - DNA, 121 bp, Ocimum basilicum
  variation (61)..(61): at position 61, “n” is either C or T
tgcttccataatcttcagccaaccaaatccaattccaaattatctcagagcttcaataggntcttgccatgacttgtgcaagtatggcggcagacgtgaactcccaccggaatccgaacaa

SEQ ID NO: 87 - DNA, 121 bp, Ocimum basilicum
  variation (61)..(61): at position 61, “n” is either G or A
ctaattctgctcgtattcctggtagtgtagcagcttctgctcgtaacttttcgcactgcantgtggaacccatagataaaacaaaactgttacataagcggaaagcacttctaaagcgtta

SEQ ID NO: 88 - DNA, 121 bp, Ocimum basilicum
  variation (61)..(61): at position 61, “n” is either T or C
tctaaaaacagcctgcacaagagtgaaaggtaatcatcactattgtattagagaacaaatntgaaaccacaatttgcctatcggaagcaaacctgctgttcctttctggtcagagtttgcc

SEQ ID NO: 89 - DNA, 121 bp, Ocimum basilicum
  variation (61)..(61): at position 61, “n” is either C or A
atgctaaagccacctgcaaaaccaccatccatggatcatgcaagtaccatccaaattcacntatccgctcagcatcaaccgacatgaaatttatgatttcccccgttgtttgtccttgctt

SEQ ID NO: 90 - DNA, 121 bp, Ocimum basilicum
  variation (61)..(61): at position 61, “n” is either C or G
aaaaatttggatcgaaacattaaatgtggattctatctctattcttcctgtgattcttctnttctgacttgatctttgaatgttcttgccgttctttgaatcttcaaaggaatttggctcc

SEQ ID NO: 91 - DNA, 121 bp, Ocimum basilicum
  variation (61)..(61): at position 61, “n” is either A or G
tgtgtttggtcacactcatttttgctgggatgcagttgttgagggcatcaggtgattttantaatgaaaataatttcagtaatgaattatgttactttatcgtatccagagttggtattcc

SEQ ID NO: 92 - DNA, 121 bp, Ocimum basilicum
  variation (61)..(61): at position 61, “n” is either T or C
aggtgcatcaaaatcaataaatgaagcctccactttaatctcagttggattcgcatccgangatgccaagctactggaggatgctgttctctgcaaaaagaaatatttcactatttatatt

SEQ ID NO: 93 - DNA, 121 bp, Ocimum basilicum
  variation (61)..(61): at position 61, “n” is either C or G
agcttggtaagtctcatatgtttatatttaaatgattgtcgtttgctggttgagttaccanatgaaataagaacccttaagaagcttgaactcctcgatattcgtgggacctcaattcgct

SEQ ID NO: 94 - DNA, 121 bp, Ocimum basilicum
  variation (61)..(61): at position 61, “n” is either A or G
gtaagtctcatatgtttatatttaaatgattgtcgtttgctggttgagttaccacatgaantaagaacccttaagaagcttgaactcctcgatattcgtgggacctcaattcgctcattgc

SEQ ID NO: 95 - DNA, 121 bp, Ocimum basilicum
  variation (61)..(61): at position 61, “n” is either G or A
tatagcttttacacccaagaatgttatccctattggatataggtgggtgtttgtatgaaanagaaatgaaaataataaggtggtaagatataaagcgaaattagtagctcaaggattttct

SEQ ID NO: 96 - DNA, 121 bp, Ocimum basilicum
  variation (61)..(61): at position 61, “n” is either G or A
ataataattaataactaaaaataaaaaaaaattgtgtgaaattaagaaaaataaatacttntaaaaaaatatccacacaaaaatggtgaagataagtcctcttttgaccatcgacagtctt

SEQ ID NO: 97 - DNA, 121 bp, Ocimum basilicum
  variation (61)..(61): at position 61, “n” is either T or G
gaaagatctaagatgatactgctatacataattgtgcaaaaaaaagggcattacagacaancatagacgcggtatgataatgttacactgtgtatgaagtaacaaagggttatagccaaga

SEQ ID NO: 98 - DNA, 121 bp, Ocimum basilicum
  variation (61)..(61): at position 61, “n” is either C or T
ctaagatgatactgctatacataattgtgcaaaaaaaagggcattacagacaatcatagangcggtatgataatgttacactgtgtatgaagtaacaaagggttatagccaagaaaaacca

SEQ ID NO: 99 - DNA, 121 bp, Ocimum basilicum
  variation (61)..(61): at position 61, “n” is either G or T
aaaaaaagggcattacagacaatcatagacgcggtatgataatgttacactgtgtatgaantaacaaagggttatagccaagaaaaaccagtataattctgctgaaaaacctatcaagaaa

SEQ ID NO: 100 - DNA, 121 bp, Ocimum basilicum
  variation (61)..(61): at position 61, “n” is either C or G
ctctttttgtgttatattccaattgaatgattgtttgcctctcttacaggccatcatgagnaccaatcaacagagtgcacggctgctttaacagagttgcttgagcaggcagttttaattt

SEQ ID NO: 101 - DNA, 121 bp, Ocimum basilicum
  variation (61)..(61): at position 61, “n” is either T or C
gaggttgtgggcgatgtgatccctagcatgcaatgcattaacagacctcacattattctcngggatgatctcatcatcgaatatctcaaacctcaactcctcactcaatccctcattcctc

SEQ ID NO: 102 - DNA, 121 bp, Ocimum basilicum
  variation (61)..(61): at position 61, “n” is either T or C
ttatgtcctttcataattaggaagaatcctactattctatctcccttttcttatgtattcntatcctatcgattcaggaagaaaagttggaacctatcaaaaacaccatggatgatccaag

SEQ ID NO: 103 - DNA, 121 bp, Ocimum basilicum
  variation (61)..(61): at position 61, “n” is either A or G
atgtcctttcataattaggaagaatcctactattctatctcccttttcttatgtattcttntcctatcgattcaggaagaaaagttggaacctatcaaaaacaccatggatgatccaagga

SEQ ID NO: 104 - DNA, 121 bp, Ocimum basilicum
  variation (61)..(61): at position 61, “n” is either C or T
gaggcttatactagaaggcttctccactcgaataaaaagattcaatcttggccagaaatanaagtatcaccaagtttgattggatctacaaaatattatacatcatcctatcaattttaaa

SEQ ID NO: 105 - DNA, 121 bp, Ocimum basilicum
  variation (61)..(61): at position 61, “n” is either G or A
aacaatttctgagcgactccaggcttctttcatcacccattacccttgagttatctgatcnagcttcatgatacatctccgagatacgtttccagaatgtcgcactattttggttagtgca

SEQ ID NO: 106 - DNA, 121 bp, Ocimum basilicum
  variation (61)..(61): at position 61, “n” is either T or A
tgcttgaagcaacgtatcaacaaattttctggtgggaccaataccatcttatggtgttctngcaaatttccgaaatgattcgtaagtcacgaggctttgtgagttgtgctatgttgtgctc

SEQ ID NO: 107 - DNA, 121 bp, Ocimum basilicum
  variation (61)..(61): at position 61, “n” is either A or G
ctctccctttttctgttatgggtgatatcttgatgtcacagttctttctatttgatttttnatttcactatatactattaaaaattaattgcaaaatgagattcaagaattagcgataata

SEQ ID NO: 108 - DNA, 121 bp, Ocimum basilicum
  variation (61)..(61): at position 61, “n” is either C or T
ttcatacgagctggatagtttggcagtttatttttggttcaaggaattcagagacaactanattgaactttttggattgaaagccggtttttcaacttccaatggcgaaggatttaatatc

SEQ ID NO: 109 - DNA, 121 bp, Ocimum basilicum
  variation (61)..(61): at position 61, “n” is either T or C
atacgagctggatagtttggcagtttatttttggttcaaggaattcagagacaactacatngaactttttggattgaaagccggtttttcaacttccaatggcgaaggatttaatatccac

SEQ ID NO: 110 - DNA, 121 bp, Ocimum basilicum
  variation (61)..(61): at position 61, “n” is either T or C
attttaatccctacatgctacagagtaggaaagggagaaaatatataaaaaatcgtggggntgacacgtggaagcatgggagccgtcgattggtaaaatccgagctgtaaccgtcgtgcct

SEQ ID NO: 111 - DNA, 121 bp, Ocimum basilicum
  variation (61)..(61): at position 61, “n” is either A or C
agttgttctaggagatgtgaaagaaaaggtgctgaactttagatttccacacacttatcanctgaatgttccttttgttagcaaagttatcacattttaacatcgtatacttttttcttta

SEQ ID NO: 112 - DNA, 121 bp, Ocimum basilicum
  variation (61)..(61): at position 61, “n” is either A or C
gttctaggagatgtgaaagaaaaggtgctgaactttagatttccacacacttatcaactgnatgttccttttgttagcaaagttatcacattttaacatcgtatacttttttctttaatat

SEQ ID NO: 113 - DNA, 121 bp, Ocimum basilicum
  variation (61)..(61): at position 61, “n” is either A or T
ttctatgcactcttttgcagattcgtaggttgttttcataacgtcttgaatcatgttcacngccctccctgctagctgcattgtcgataagatgaaccagttttccatcataggtttatca

SEQ ID NO: 114 - DNA, 121 bp, Ocimum basilicum
  variation (61)..(61): at position 61, “n” is either T or G
tactgagttgctctatcaatgggggcgatacagcttcggcaactgctcgtcgcctgcctcnaccacccccatttctggcgccgtttcttcttgtgggccccgaattccagctatggcctgt

SEQ ID NO: 115 - DNA, 121 bp, Ocimum basilicum
  variation (61)..(61): at position 61, “n” is either T or C
ccacaaattgaccatgttgatgagtgattctctgggtggtaatgccaaaacattgatgttngtcaacgtgtctccagcagagtcaaatctggacgagacctacaactctttaacgtaagct

SEQ ID NO: 116 - DNA, 121 bp, Ocimum basilicum
  variation (61)..(61): at position 61, “n” is either T or G
ttgttttagtctaacaaaaaagatgcagcatcaataagcctgtcattttcaggcgctggtnggggcaactgcagctactagaaatatagaacaagcacttcaaatggaaaattgcatgaag

SEQ ID NO: 117 - DNA, 121 bp, Ocimum basilicum
  variation (61)..(61): at position 61, “n” is either A or C
gccaagtaagaggcttaatgtccagaaatttaatcttggtggcaccccaaggaaagtactntatcataatgaaagcaggttattgcttgtcatgaggacagagttggataatgagatcttc

SEQ ID NO: 118 - DNA, 121 bp, Ocimum basilicum
  variation (61)..(61): at position 61, “n” is either G or C
atgtccagaaatttaatcttggtggcaccccaaggaaagtactatatcataatgaaagcangttattgcttgtcatgaggacagagttggataatgagatcttcccatctgatgtttgttg

SEQ ID NO: 119 - DNA, 121 bp, Ocimum basilicum
  variation (61)..(61): at position 61, “n” is either G or A
cactgagcaatcctgcaatagatccatgtagcaatcagagagcatagtaagatatgaaatnacgaaaaaactctccaatcttaactccctcatcaagttctaagaatgcctagaagaatga

SEQ ID NO: 120 - DNA, 121 bp, Ocimum basilicum
  variation (61)..(61): at position 61, “n” is either C or T
ctatttctttcttctatctgcagaagctttgccattcttagatatgtggtttgtcaactcntaaatgttgtgctttcttcgtcaggttttcgatgggcatggaggaaaacatgctgccgac

SEQ ID NO: 121 - DNA, 121 bp, Ocimum basilicum
  variation (61)..(61): at position 61, “n” is either T or C
ttatctgtttcaagaaaatactgaaccaacaccacctagttaccgaatccagtggaaaaantatactgatgagttagctctgatgctacaggtgcaacatcatttcttttcacggagtata

SEQ ID NO: 122 - DNA, 121 bp, Ocimum basilicum
  variation (61)..(61): at position 61, “n” is either C or T
ataataattctcatgcgccgtgtccaagaaaattggcatccttctccatctgcaagtaatnataaaggttatgttccatctcgtgaaacgttgaaagctatgaaagatgtagagagaatta

SEQ ID NO: 123 - DNA, 121 bp, Ocimum basilicum
  variation (61)..(61): at position 61, “n” is either T or C
agctgttcgtattggaccgccaccggctcggctgagctacttgagagctgactccatcatngaagccgcttctagaaccggcgcacaggttgcagattatgctgatttctacgttttaatg

SEQ ID NO: 124 - DNA, 121 bp, Ocimum basilicum
  variation (61)..(61): at position 61, “n” is either G or T
attgactatgatgagactttttcaccggttaccataatcaaatccattcaaattcttttanccattgctactcacatggacctagaagtttgacaaatggatgtaaaaaccgtgtttctac

SEQ ID NO: 125 - DNA, 121 bp, Ocimum basilicum
  variation (61)..(61): at position 61, “n” is either T or C
agtaaacatatcaggtaaagtagttctgccagctaaatattctctggcttatcctttgacnataatctcataattgtcactttctcaggatcttttactgcgatgattctgatggatagat

SEQ ID NO: 126 - DNA, 121 bp, Ocimum basilicum
  variation (61)..(61): at position 61, “n” is either T or C
gaaactttggaaaaacttgatttagatgattttattaatgatttttctttccaaaatgctngaagatctaaattatttcaataatttattttatattcatttctttacatttttagtagta

SEQ ID NO: 127 - DNA, 121 bp, Ocimum basilicum
  variation (61)..(61): at position 61, “n” is either A or G
gttggaaatgatacacaaaggaaaaagcataatataattattatgactgtcgtagttacangagaagaaggcaaaccaacatttccttggaaggaataagagtttttgagaaaccacacag

SEQ ID NO: 128 - DNA, 121 bp, Ocimum basilicum
  variation (61)..(61): at position 61, “n” is either G or C
gtgcaaccacgtgcttctgacctcctacttttcttgcttgccctgcgagggtaaaacctcntaccgcctctgctgcaactgctacagtcgacgtaagttcgatcaccatcacccctcccac

SEQ ID NO: 129 - DNA, 121 bp, Ocimum basilicum
  variation (61)..(61): at position 61, “n” is either A or G
gcggaacggtttgattcacttctactgcgagagtggcgaactgagctgcgcgaatcgagtntttcgcgagaatgagatgagggatgtggtttcttggactagtatcattgatgggaatgtg

SEQ ID NO: 130 - DNA, 121 bp, Ocimum basilicum
  variation (61)..(61): at position 61, “n” is either T or C
tggattctcttccatgctgtgatgcacatagtagtatgtgttcatcggcggataatattgncggtagggcattgctgtcccaagcacgagctccggttctcccttcttctcctcatctttc

SEQ ID NO: 131 - DNA, 121 bp, Ocimum basilicum
  variation (61)..(61): at position 61, “n” is either G or C
agctccaaaaccacgggtgtaagatgtttcgcagctgccattaatactattcgacccaccntgctacctcctgcaaaagttaatcatttacttctggtgtttaaacacatgcataactgag

SEQ ID NO: 132 - DNA, 121 bp, Ocimum basilicum
  variation (61)..(61): at position 61, “n” is either C or T
atattaagaaaaatgatcaagtcaatggaataaaataaattattaattgtgggttgcaacnttatgaggtgcaagaaatgaaagtaaaatgtccaaaaaaatatgaagattacacaaaaat

SEQ ID NO: 133 - DNA, 121 bp, Ocimum basilicum
  variation (61)..(61): at position 61, “n” is either C or A
cacctgctccgacagcctcgacaatgagctatcatttatgtcgaaaaccacctcctggcanaggcggttgaacatctccgacacctcgccgtcgctccccagccagttctctattatcccg

SEQ ID NO: 134 - DNA, 121 bp, Ocimum basilicum
  variation (61)..(61): at position 61, “n” is either G or A
cctgctccgacagcctcgacaatgagctatcatttatgtcgaaaaccacctcctggcacangcggttgaacatctccgacacctcgccgtcgctccccagccagttctctattatcccgct

SEQ ID NO: 135 - DNA, 121 bp, Ocimum basilicum
  variation (61)..(61): at position 61, “n” is either A or G
tttaatataaaaccaaactcttaatctcaaaaacatcattaagacagtagttaatttcttntagttatttgtagtgagctgaacctcataaacagtaccagattcgataatttaatttgtg

SEQ ID NO: 136 - DNA, 121 bp, Ocimum basilicum
  variation (61)..(61): at position 61, “n” is either G or A
caaaaacttgatttcaagaagataaagattctgtgtttctggtagtaaaatgagaggtganataagaaagaagctgcatttgagcaagatctactcattcaaacgtggtagaggaggattc

SEQ ID NO: 137 - DNA, 121 bp, Ocimum basilicum
  variation (61)..(61): at position 61, “n” is either T or C
tttatgaccggttgatttccttgcttcaccctcttcttggctggaaagtcgctcgggcttnggcgattttgcttcggatgcctttaaatcagcctccatgttgtcgtggggattgatttgg

SEQ ID NO: 138 - DNA, 121 bp, Ocimum basilicum
  variation (61)..(61): at position 61, “n” is either G or A
ttatgaccggttgatttccttgcttcaccctcttcttggctggaaagtcgctcgggctttngcgattttgcttcggatgcctttaaatcagcctccatgttgtcgtggggattgatttggt

SEQ ID NO: 139 - DNA, 121 bp, Ocimum basilicum
  variation (61)..(61): at position 61, “n” is either C or G
tttgccttcccgctctctcacgacattgttagcgaactcttcgagtatccaatcatcttcntcgtcaccatcaaaatctccttcgagtactacgatttctacctttgcagaggaaccaggg

SEQ ID NO: 140 - DNA, 121 bp, Ocimum basilicum
  variation (61)..(61): at position 61, “n” is either T or G
aatccgcgtacaaagaagaagaaagaaaaaaggttgttgctggtgcttatgtttgattaangatggattgactgaacttttcattttcaattgattttaattaaataaatattcatctctt

SEQ ID NO: 141 - DNA, 121 bp, Ocimum basilicum
  variation (61)..(61): at position 61, “n” is either A or C
tatgacatgactaatatcagccaaatcgttgttaccctgccaagaaggaacacagaaagangatgatctacaccttgttaattttattttgattgtcttgataaaaactacaagatattaa

SEQ ID NO: 142 - DNA, 121 bp, Ocimum basilicum
  variation (61)..(61): at position 61, “n” is either C or A
ggcgccaatattgacgtcatctaccataagcaataaagtttgggcttttgttacttgacancaccacctggcattgttgctgctatcttctgtccttggatctaccattgtcgtatctgcc

SEQ ID NO: 143 - DNA, 121 bp, Ocimum basilicum
  variation (61)..(61): at position 61, “n” is either A or G
cgccaatattgacgtcatctaccataagcaataaagtttgggcttttgttacttgacaccnccacctggcattgttgctgctatcttctgtccttggatctaccattgtcgtatctgcctt

SEQ ID NO: 144 - DNA, 121 bp, Ocimum basilicum
  variation (61)..(61): at position 61, “n” is either T or C
tttacatctgtcttctgacacagtggatcctccttcgccgacttcaatgcatgctgttttnctgttgcagaaatctccaacttccaggcctgaaacgtctccagcagcggatttctgctgc

SEQ ID NO: 145 - DNA, 121 bp, Ocimum basilicum
  variation (61)..(61): at position 61, “n” is either T or C
atcacacccgtgggttctagcagtacttccaagtggagaggcaataggagtccttgcaganacaactagacactgtgaggttgtgatctttgagttatttaatgagctggtttgttttgca

SEQ ID NO: 146 - DNA, 121 bp, Ocimum basilicum
  variation (61)..(61): at position 61, “n” is either T or C
tactgaaatgggttttctcgtgttaacttattcatatcaacttccgaaactaatttttttnaatctaagcaggttatttatggtgatacagattcaatcatgatttacagtggacttgatg

SEQ ID NO: 147 - DNA, 121 bp, Ocimum basilicum
  variation (61)..(61): at position 61, “n” is either A or T
ctctttgtccatgcctcgtgcaccggcgttcactcccttcttctgctcggggcctgacaanacatgttctaaaagtatcaaagtttagagtagtagtagagctttgttaatcaagtaacac

SEQ ID NO: 148 - DNA, 121 bp, Ocimum basilicum
  variation (61)..(61): at position 61, “n” is either C or T
accttgcaactactccaaagatatgatgaattaatcgttgcatgcacaatgtcttgccgtntcccttctgctattactggcaaaaatttgtcgtaaatcacctcccaatactatagtttta

SEQ ID NO: 149 - DNA, 121 bp, Ocimum basilicum
  variation (61)..(61): at position 61, “n” is either T or A
ttgagggatggtgaggtgaaatgatttaatcatgtgaattggtagaaaactagttacctanaaatagaccaaaacaatcacaagtatgagtgatggttgatatgaataattattctaaatc

SEQ ID NO: 150 - DNA, 121 bp, Ocimum basilicum
  variation (61)..(61): at position 61, “n” is either C or T
gattatgttgtcgataatcatatcggaaaaacagatgaatatagcaggcatttggattacncctcaggggcccttgatgatcaggctactatttcacatgtacaccaggaaggaaattcaa

SEQ ID NO: 151 - DNA, 121 bp, Ocimum basilicum
  variation (61)..(61): at position 61, “n” is either A or C
cagaactccccagggtttacatacagaaaaaaaaaaccacaaaaaactcttgtaagtgaanaataatgcatcaagccactaagcagcagaaccaaaaagactacaaactgttgggtgttta

SEQ ID NO: 152 - DNA, 121 bp, Ocimum basilicum
  variation (61)..(61): at position 61, “n” is either A or G
cctctgcgaagcctccatccttcaacagctatccaccaatctcacttcctcgacttattantcgatcaataaatcctctagtttatcctcctgttctccgctaaaagagataacgatctca

SEQ ID NO: 153 - DNA, 121 bp, Ocimum basilicum
  variation (61)..(61): at position 61, “n” is either C or G
atagaaagagagagaatttctgaagctgtctgagcgtgaaatgtgcccattgtattgtgtnttgctttcttaaaacacacactgcttttagcctcagaaaacaacaatttttggaagctca

SEQ ID NO: 154 - DNA, 121 bp, Ocimum basilicum
  variation (61)..(61): at position 61, “n” is either T or C
tcatcattaggacttttgaagttcgtgaatgccaattaatgtaccacaatttactcgaagntcctatataattaataaataaaatcttcagactcattaactcctaaaagtcaccgatgca

SEQ ID NO: 155 - DNA, 121 bp, Ocimum basilicum
  variation (61)..(61): at position 61, “n” is either C or T
atttgttgaaaaaaatggtatttcattggacaatagatatgttgttccatacaattgatancttctgatgaaatatggtggacatatcaatgtcgaacggtgcaatcaatcaagatcgatc

SEQ ID NO: 156 - DNA, 121 bp, Ocimum basilicum
  variation (61)..(61): at position 61, “n” is either G or A
agaagtatcttttctttctcctcctcttcaacatcatggtaaacaaactcagattcagccntgtagcctaatcttttcatttcagccattagttcctccagtttcttgtatattttggatg

SEQ ID NO: 157 - DNA, 121 bp, Ocimum basilicum
  variation (61)..(61): at position 61, “n” is either A or C
aatgtattcattgtacaacttgcgtattgcctcacaaactgtggtaaccaacataagtccntcagatccagtgcaaacattggaagcacaccactcaatcaattttatcttattactagga

SEQ ID NO: 158 - DNA, 121 bp, Ocimum basilicum
  variation (61)..(61): at position 61, “n” is either G or A
gaagcccctttttggcggcggtagtggtagtgcttctggttttcgcgagcatcagcacgtncacccctctcccgtcatgtcatccataaatggtgggccgtttcaccaccatcgcgttggt

SEQ ID NO: 159 - DNA, 121 bp, Ocimum basilicum
  variation (61)..(61): at position 61, “n” is either T or C
aaatctgggcagaggtacaaagatcactctctttctgaaggaggatcagctcgagtatctngaggagcgtaggcttaaggatttaatcaagaagcactctgaattcatcagttatcccatc

SEQ ID NO: 160 - DNA, 121 bp, Ocimum basilicum
  variation (61)..(61): at position 61, “n” is either T or G
gcttaaggatttaatcaagaagcactctgaattcatcagttatcccatctctcagtgggtngagaaaaccacggaaaaggaaatctctgatgatgaggatgaggatgaggatgaggacgac

SEQ ID NO: 161 - DNA, 121 bp, Ocimum basilicum
  variation (61)..(61): at position 61, “n” is either G or T
tttaaatgatggtggaaatgatgaaccttcagtccacaacacttcaaattctaatggtgangagcaaatctagagaaggtcaacaagggagaaatgacagccgagttacctcgaagactat

SEQ ID NO: 162 - DNA, 121 bp, Ocimum basilicum
  variation (61)..(61): at position 61, “n” is either T or C
cgggccaactatatatgctagaagaggagatctgctcatagtcgatgttatcaatcgtgcnaatcataatataaccatccactggtaaaactagttatttaaaactgaacatcttttgatg

SEQ ID NO: 163 - DNA, 121 bp, Ocimum basilicum
  variation (61)..(61): at position 61, “n” is either A or G
gggccaactatatatgctagaagaggagatctgctcatagtcgatgttatcaatcgtgctnatcataatataaccatccactggtaaaactagttatttaaaactgaacatcttttgatga

SEQ ID NO: 164 - DNA, 121 bp, Ocimum basilicum
  variation (61)..(61): at position 61, “n” is either A or G
ctatatatgctagaagaggagatctgctcatagtcgatgttatcaatcgtgctaatcatantataaccatccactggtaaaactagttatttaaaactgaacatcttttgatgatattatt

SEQ ID NO: 165 - DNA, 121 bp, Ocimum basilicum
  variation (61)..(61): at position 61, “n” is either C or T
aagaggagatctgctcatagtcgatgttatcaatcgtgctaatcataatataaccatccantggtaaaactagttatttaaaactgaacatcttttgatgatattattaaatcactagaca

SEQ ID NO: 166 - DNA, 121 bp, Ocimum basilicum
  variation (61)..(61): at position 61, “n” is either G or A
ggtcattatcaacagtctacaaaatttaatagaaatcaaatttgtgtcgatacggaaaaangaaaacaaaaattcaaggcagtgatatgttaaatattttgaaaattagaccttgacgaga

SEQ ID NO: 167 - DNA, 121 bp, Ocimum basilicum
  variation (61)..(61): at position 61, “n” is either T or G
accttccaaaaattgcatgtgccttctcatttatactgtatctttatttttcttggcagantaaaaggtaaattatacaaatgaacttcccacaatgatcaaatatacagttatttttcac

SEQ ID NO: 168 - DNA, 121 bp, Ocimum basilicum
  variation (61)..(61): at position 61, “n” is either A or G
gaagatggtgcaacggagaaactcaaagaggtcaacatcagacgctgttgcaagctaaaanactttcccagaagactgttgcagcagacaactttcaaggaattgatcttaaacgacatgc

SEQ ID NO: 169 - DNA, 121 bp, Ocimum basilicum
  variation (61)..(61): at position 61, “n” is either A or G
atcacattgtgatgaacaagaatgaaccaaaaaataaaaagaaaactagcattgaggtctntacattggattatcttctccaatcaaaagagaccttaagatggcttcacaaacagcagaa

SEQ ID NO: 170 - DNA, 121 bp, Ocimum basilicum
  variation (61)..(61): at position 61, “n” is either A or G
tgaacaagaatgaaccaaaaaataaaaagaaaactagcattgaggtctatacattggattntcttctccaatcaaaagagaccttaagatggcttcacaaacagcagaatcataaaccagc

SEQ ID NO: 171 - DNA, 121 bp, Ocimum basilicum
  variation (61)..(61): at position 61, “n” is either G or A
aaaaaggggaagataggtgatcttactggagcggatgagaagattgatgaatgggagaatntcgacagtgcaattaaggagttcgccaaacagtttgaagggatgactggaaacaagtttg

SEQ ID NO: 172 - DNA, 121 bp, Ocimum basilicum
  variation (61)..(61): at position 61, “n” is either C or T
atcaatgaagtgatcgacatcaaagatttcctcaaacgtgctgcaagaaaagaaaacgggntttccttgttcagtttaaacgatccaaaacgaaaggaattaagaactagaactctgaacc

SEQ ID NO: 173 - DNA, 121 bp, Ocimum basilicum
  variation (61)..(61): at position 61, “n” is either G or A
atagcacctcgtcatacaaccttatccgtgtatttctgtcgagtcatcacctcacctggtnctgacacgtggatcacaatatattccgtatctttcagttcatctagaaaaggggtgatag

SEQ ID NO: 174 - DNA, 121 bp, Ocimum basilicum
  variation (61)..(61): at position 61, “n” is either T or C
acaaatccatcatataactactattattattatcataatatattttatttactaatctatntactatctatattttttaatttattttatttactaatatatttgtattttatatatgtgt

SEQ ID NO: 175 - DNA, 121 bp, Ocimum basilicum
  variation (61)..(61): at position 61, “n” is either T or C
ttagtggctgaggaagaatggctatctctcagccaaagtcttgatattacagcacacgtgntgcgtcttggaggtatatatggccctgggagaaggtttgtattatttaaaatataaacac

SEQ ID NO: 176 - DNA, 121 bp, Ocimum basilicum
  variation (61)..(61): at position 61, “n” is either T or C
aataaaatttagtatatcacgaaaaataatatatgtttgcatcttatttaggtatttgggngaacttaaatcctatgttcgaaacaaaaataaacctgaaggttccataacccacggatat

SEQ ID NO: 177 - DNA, 121 bp, Ocimum basilicum
  variation (61)..(61): at position 61, “n” is either G or A
gaccgctgccatggaaactctagaccccacaacttttcaaggtggattcattcttgaaaanattagaggccctctctctcaaggccacctccaactcataaacacaaaccctaacgaaaat

SEQ ID NO: 178 - DNA, 121 bp, Ocimum basilicum
  variation (61)..(61): at position 61, “n” is either G or A
cttttaattgttcagtttgtgtactttcaggaatattcttcacatagagggccttgacctngataaaaattaaaaaagttataaaaataatgccacgacaagcactaaacaactgagattc

SEQ ID NO: 179 - DNA, 121 bp, Ocimum basilicum
  variation (61)..(61): at position 61, “n” is either A or T
gaccaagtatcaaaatgtaatgacgtcaatattttcaataagttccttgaagcatgtgatnttgtggtaagtgtacccaatactgcgtttgtatcataacaagatcattattagatcgcac

SEQ ID NO: 180 - DNA, 121 bp, Ocimum basilicum
  variation (61)..(61): at position 61, “n” is either A or T
ttctaaaccaaaaaataatgttttttttccttccaatcatacaccgaacacaatcctaagncaaaaaaaaaaaagtagattttacactaaaaataaacctaaatttgatgttacactattg

SEQ ID NO: 181 - DNA, 121 bp, Ocimum basilicum
  variation (61)..(61): at position 61, “n” is either T or C
gatgcacgcgccgaatcctgttaataggtactttaggcatgtggtccattgcaaggagtgnacgagtgtgatgagatattgtggtgtatggagaaaggctttctttgcggctgcggttgtt

SEQ ID NO: 182 - DNA, 121 bp, Ocimum basilicum
  variation (61)..(61): at position 61, “n” is either A or T
tgatatttattattatcatataaaaattagaagataaaaagtgttaggataacatctaaantgcattattgataagtacaaatcttatttattatatcaaaacaaataaaattaagttaat

SEQ ID NO: 183 - DNA, 121 bp, Ocimum basilicum
  variation (61)..(61): at position 61, “n” is either T or C
gtgctatcagttttgtaagcgtatcttgatactttatatctgcaccagtaaccattaaatnttttttagcctgtttatttcaattagaaatagtttctacacttaggcaagttccatcaat

SEQ ID NO: 184 - DNA, 121 bp, Ocimum basilicum
  variation (61)..(61): at position 61, “n” is either T or C
aatttcttttataaaaagagtccacctaactccattagtatgaagccttttgggagatgtncaaaaacaaatccgtgcgaacttgtgttcggcccaaagcggacaaatcatactaatgtcg

SEQ ID NO: 185 - DNA, 121 bp, Ocimum basilicum
  variation (61)..(61): at position 61, “n” is either G or T
cttgggtgcacaaggaagagttgattgctttggagcaaacatcagctatcaaatcaccttnacaacaccataagaatgtgaataaaagaacgggtaatggagagagggaagccatcctttt

SEQ ID NO: 186 - DNA, 121 bp, Ocimum basilicum
  variation (61)..(61): at position 61, “n” is either A or G
tagtcgagagaaagttgaaaacttgaagaaaagatgcacttactctatgaggagtacaganaatgaggcattgcttcatcttttggagttcctacttcgcaagttattgcctatgatattt

SEQ ID NO: 187 - DNA, 121 bp, Ocimum basilicum
  variation (61)..(61): at position 61, “n” is either T or C
acactgatctttagaaagaggaacaccgtgtctgaaaggtagcaaacatttctgggtgttntgcatggtgaaacgagcaattacagtatcaatataggatgtctgtcttaagctcaacatc

SEQ ID NO: 188 - DNA, 121 bp, Ocimum basilicum
  variation (61)..(61): at position 61, “n” is either G or A
atatgaagaaataaactttgagacatttgaaaacgcctaagaaatatttcttcgttatagntgggattttctgaaaaataatcataaaaaagtctttaggcagctgcttctcgaccacgat

SEQ ID NO: 189 - DNA, 121 bp, Ocimum basilicum
  variation (61)..(61): at position 61, “n” is either T or A
ttgcactcttcactcgaggtggataaaattattattatttttgcaaagaaagaatgttttnataaatgtttttatgtccaatatgtgatattgcgtggttgatatatagttgaagaaatta

SEQ ID NO: 190 - DNA, 121 bp, Ocimum basilicum
  variation (61)..(61): at position 61, “n” is either A or G
acaaggaagctgacaagttggagtcaatgacagaagtaatgttatttgtaggatatccaantgaaacaaaaggtggttatttctatagttctaaggatcagaaggtcgttgttagcaccaa

SEQ ID NO: 191 - DNA, 121 bp, Ocimum basilicum
  variation (61)..(61): at position 61, “n” is either T or G
tcgcctgctagtcttatgatagaagctctctttggatgccgttctagccatacctgcatgnaaaaaaatgtgtgattttgttaagaataaccaccatagcagaagataatttactcaagtg

SEQ ID NO: 192 - DNA, 121 bp, Ocimum basilicum
  variation (61)..(61): at position 61, “n” is either C or A
ttcgaagaatgtggggccggtttcgttttgtgaacggcctggaggagacattcatacatanaatgaagatttccatgttgcgaggaaaagtctatatagttgtaagaggagatatgtacct

SEQ ID NO: 193 - DNA, 121 bp, Ocimum basilicum
  variation (61)..(61): at position 61, “n” is either A or G
cggagggtattggatgttgatggggttttgattgtttatgtttcaggaagtacaagattanagatgatatttattcacttatgtggtcttcaaggaacctggggcaggtttgtgattgtct

SEQ ID NO: 194 - DNA, 121 bp, Ocimum basilicum
  variation (61)..(61): at position 61, “n” is either G or A
agccccagagacttttgatctttttgatgatctcatattactctctgaaggtcacctcgtntatcaagggccccatgatgaagttattgagttttttcagtcgcttggtttcttgttacct

SEQ ID NO: 195 - DNA, 121 bp, Ocimum basilicum
  variation (61)..(61): at position 61, “n” is either G or A
gatctttttgatgatctcatattactctctgaaggtcacctcgtgtatcaagggccccatnatgaagttattgagttttttcagtcgcttggtttcttgttacctcctcgtaagagtgtcg

SEQ ID NO: 196 - DNA, 121 bp, Ocimum basilicum
  variation (61)..(61): at position 61, “n” is either G or A
gccagggtcaccaagcaggagtacatttatatctcctcgtagacggtgttttccttcaacnttcttctcttggccaccaaacatggcaagagctattgcagtctttatgtcctcatgacca

SEQ ID NO: 197 - DNA, 121 bp, Ocimum basilicum
  variation (61)..(61): at position 61, “n” is either G or T
tgccctgaagaaatcatttgaagggtggtctgcgcagacgacgatttttcccattgcaagngcctcggctgtagcagtgcaaagcacgtcgcttacactgggatttacaaagattttgtaa

SEQ ID NO: 198 - DNA, 121 bp, Ocimum basilicum
  variation (61)..(61): at position 61, “n” is either A or G
agatgagtcacataacaaagatttattaaattttgccaaaatggacttcgatatattgcanaagatgcatcaagaagaggcacgtcaagtcacaaggttagcatcatcagtaacatcatta

SEQ ID NO: 199 - DNA, 121 bp, Ocimum basilicum; identical to SEQ ID NO: 198
  variation (61)..(61): at position 61, “n” is either A or G

SEQ ID NO: 200 - DNA, 121 bp, Ocimum basilicum
  variation (61)..(61): at position 61, “n” is either G or A
gatgtctactcaagctcgtggaggttacgagtacttcattactttcattgacgattactcnaggtttggatacgtttacctgctttgctgcaagtacgaatgctttgagaaattcaaagac

SEQ ID NO: 201 - DNA, 121 bp, Ocimum basilicum
  variation (61)..(61): at position 61, “n” can be either T or G
aatttggataaaaagaaatgtttgattgttgtgatttcatgatgtaaaatgagagttgtanttataaatgaaaatttatgaactttgatatgaaaatgtgtggaggtcaaattgacatgga

SEQ ID NO: 202 - DNA, 121 bp, Ocimum basilicum
  variation (61)..(61): at position 61, “n” is either A or G
aaaagaaatgtttgattgttgtgatttcatgatgtaaaatgagagttgtatttataaatgnaaatttatgaactttgatatgaaaatgtgtggaggtcaaattgacatggattgtgtgctt

SEQ ID NO: 203 - DNA, 121 bp, Ocimum basilicum
  variation (61)..(61): at position 61, “n” is either A or G
agaaatgtttgattgttgtgatttcatgatgtaaaatgagagttgtatttataaatgaaantttatgaactttgatatgaaaatgtgtggaggtcaaattgacatggattgtgtgcttttt

SEQ ID NO: 204 - DNA, 121 bp, Ocimum basilicum
  variation (61)..(61): at position 61, “n” is either C or T
tttcccgatgtgtggcatgaacttgggattcaaccgtgaactaatcgggcccgcaatgtantttggactaatgggagacggccagccaatagggcgatacgacgatatgtgggctggttgg

SEQ ID NO: 205 - DNA, 121 bp, Ocimum basilicum
  variation (61)..(61): at position 61, “n” is either A or T
caaccgtgaactaatcgggcccgcaatgtactttggactaatgggagacggccagccaatngggcgatacgacgatatgtgggctggttggtgcaccaaggtagtaaccatgttgccatta

SEQ ID NO: 206 - DNA, 121 bp, Ocimum basilicum
  variation (61)..(61): at position 61, “n” is either T or G
tcattcttctggtttaatcaatttttgccagaagcaagtaactgtcaaatcatcggttctngaagtgctgcttgagatcacaaaatgttgtgatctctatcttatggaaagagtggtggat

SEQ ID NO: 207 - DNA, 121 bp, Ocimum basilicum
  variation (61)..(61): at position 61, “n” is either C or G
atttttgccagaagcaagtaactgtcaaatcatcggttcttgaagtgctgcttgagatcanaaaatgttgtgatctctatcttatggaaagagtggtggatgatgaaagtgaagtaagacc

SEQ ID NO: 208 - DNA, 121 bp, Ocimum basilicum
  variation (61)..(61): at position 61, “n” is either T or C
ccagaagcaagtaactgtcaaatcatcggttcttgaagtgctgcttgagatcacaaaatgntgtgatctctatcttatggaaagagtggtggatgatgaaagtgaagtaagacctactatt

SEQ ID NO: 209 - DNA, 121 bp, Ocimum basilicum
  variation (61)..(61): at position 61, “n” is either T or C
aactgattcggcaatgggaactttggcagttgagccaaagaccatgtgcccgttcgactangtgacattagggctcaaggacccaatcaaggactacctcggaaagccccgagttctgtca

SEQ ID NO: 210 - DNA, 121 bp, Ocimum basilicum
  variation (61)..(61): at position 61, “n” is either T or C
ttgctggctcttgctagcgctgtttgtgctgcctgtggttgctgctgcactttcattggangaaggtctacccacgtcccttagggtattttggggtgaaggatttggacctgcaacaggg

SEQ ID NO: 211 - DNA, 103 bp, Ocimum basilicum
  variation (61)..(61): at position 61, “n” is either C or A
ggtgatccccatcaccatctgcaccagcaggcagctcacaaagttccccagcgatatcgangccatgcacagagagctcccgaagctcttgatcccatcgggc

SEQ ID NO: 212 - DNA, 121 bp, Ocimum basilicum
  variation (61)..(61): at position 61, “n” is either G or A
ttagatgcctcattaaaaactttcactaagaaaatccaaatggaaaaaaacttagtaaggnaaaaagagtacatctattcagcttgccgggcttaatcttctggattacgtttagtttgtc

SEQ ID NO: 213 - DNA, 121 bp, Ocimum basilicum
  variation (61)..(61): at position 61, “n” is either C or T
tagagtggaaagttcacgaatatctacgcccgcctcatccattggtgggagcaagagaatnaccgagcctctctcagcgaaaaagccatcaaacaaacaagactcttccagaaatcagaat

SEQ ID NO: 214 - DNA, 121 bp, Ocimum basilicum
  variation (61)..(61): at position 61, “n” is either T or A
tacaaatcttaagcagaatttggggaatactagtaggttcttgaaatttgcaaggtgtgtntggaattaaattattggcattacagtaataagttttcctcaactaatttgagatttatac

SEQ ID NO: 215 - DNA, 121 bp, Ocimum basilicum
  variation (61)..(61): at position 61, “n” is either G or A
tttgaaggaagctaagaggtcgatgtcagagtcgaaatcgagagtgaatgaactcgagtcnatgaagagtgaagctcagagcactcaacaagaggttgagaagaggttaagggaagctgaa

SEQ ID NO: 216 - DNA, 121 bp, Ocimum basilicum
  variation (61)..(61): at position 61, “n” is either G or A
gattgcaggtatttcgaaaagtttctataggctatttgactttcacagttttcctcgaacnaaagggtttattcgatattttgttgttgcacgtttcagaatacaacatcaatagataatc

SEQ ID NO: 217 - DNA, 121 bp, Ocimum basilicum
  variation (61)..(61): at position 61, “n” is either A or G
attgcaggtatttcgaaaagtttctataggctatttgactttcacagttttcctcgaacgnaagggtttattcgatattttgttgttgcacgtttcagaatacaacatcaatagataatca

SEQ ID NO: 218 - DNA, 121 bp, Ocimum basilicum
  variation (61)..(61): at position 61, “n” is either C or G
ttttagatgttatacctaaattaatacataatctctgatgtagaaatagccgacacaaacnagttgcataatactatggtaaaatttagcaattatatattagataaaatcaaaaaactta

SEQ ID NO: 219 - DNA, 121 bp, Ocimum basilicum
  variation (61)..(61): at position 61, “n” is either G or A
aattaatacataatctctgatgtagaaatagccgacacaaaccagttgcataatactatgntaaaatttagcaattatatattagataaaatcaaaaaacttacttatattactatttaaa

SEQ ID NO: 220 - DNA, 121 bp, Ocimum basilicum
  variation (61)..(61): at position 61, “n” is either G or C
cttgcgctcatgctttctctatttcgggatgttcccggaagactacgaaattcccgtgtcnaaattgatccaaatgtggattgcagaaggattcatacaacaggatgactcttgcagtttg

SEQ ID NO: 221 - DNA, 121 bp, Ocimum basilicum
  variation (61)..(61): at position 61, “n” is either C or T
tgattatgggacatgtttgcgttttttaggatcattttcgcgatcagctgtgaattacaangctggcgcacagacggtcgtctcaaacatagtaatggctgcagccgtgcttgtgacattg

SEQ ID NO: 222 - DNA, 121 bp, Ocimum basilicum
  variation (61)..(61): at position 61, “n” is either T or A
gatttttagacgcaaagaaaaaaattgaacagaaggaatataaaagatgaaaatcttgttncacaccttgtccttgatcatttgccagatgaccctcgtcatctcatctcctgaaaacgca

SEQ ID NO: 223 - DNA, 121 bp, Ocimum basilicum
  variation (61)..(61): at position 61, “n” is either C or A
aggtaattttcttttaaaccattattatgatccttcaatcattggttgttactgctgtttnagttgtttccacaagcatcttctgcaacacagtgctgatggggaatgtaagatttcattg

SEQ ID NO: 224 - DNA, 121 bp, Ocimum basilicum
  variation (61)..(61): at position 61, “n” is either G or T
aaaccattattatgatccttcaatcattggttgttactgctgtttcagttgtttccacaancatcttctgcaacacagtgctgatggggaatgtaagatttcattgcagaaagaggtacca

SEQ ID NO: 225 - DNA, 121 bp, Ocimum basilicum
  variation (61)..(61): at position 61, “n” is either G or A
atcatggtgaccaaggtaaggccacagttagatactctgcctacactacttcttcaagctncgtgtaagaaaaattaattgtaaatgtcgatacaggtggatgagccttcaaaatatggtg

SEQ ID NO: 226 - DNA, 121 bp, Ocimum basilicum
  variation (61)..(61): at position 61, “n” is either G or A
catggtgaccaaggtaaggccacagttagatactctgcctacactacttcttcaagctgcntgtaagaaaaattaattgtaaatgtcgatacaggtggatgagccttcaaaatatggtgtt

SEQ ID NO: 227 - DNA, 121 bp, Ocimum basilicum
  variation (61)..(61): at position 61, “n” is either A or C
agacttcccatttctggccacaaactgtgcagtgagtttaattatatccaattcctccccngtaatcccttcaggaagcctaacggtatattgctctgcctccggtggctcaagaacctta

SEQ ID NO: 228 - DNA, 121 bp, Ocimum basilicum
  variation (61)..(61): at position 61, “n” is either C or T
ctgattactcatgtgtcgttggattttccgtcattggtgttaggtgattttaattgtgtgntccatcccgatgaatgtgtgggaggatgtgctcctcgcaagagggaatacacaaagtttt

SEQ ID NO: 229 - DNA, 121 bp, Ocimum basilicum
  variation (61)..(61): at position 61, “n” is either A or C
cttagttttcactctctcataattcgctccttcttaattgttaaaattgtcatcgccacantgaatggatcatgctactggtgttggtattatttagaccgaaatgaccactgctgagaga

SEQ ID NO: 230 - DNA, 121 bp, Ocimum basilicum
  variation (61)..(61): at position 61, “n” is either C or T
caaactgaaatgttataaaccctaaatgagagtattgaaccttaattgggatatcataaancctaattgggatgtaataaaaccctagctaagattaaatgaaacttaaattttatttttc

SEQ ID NO: 231 - DNA, 121 bp, Ocimum basilicum
  variation (61)..(61): at position 61, “n” is either A or G
aatagctatttcctcttgaccaaaaaacagtttctggaatccctcagaaccaggatttatnattccggcaacagaaggtgtttcccttcctggattagcaagaataagaagaaaacattta

SEQ ID NO: 232 - DNA, 121 bp, Ocimum basilicum
  variation (61)..(61): at position 61, “n” is either C or T
tacttgttgttctccagttgaactgtattgtgaaacatgaataataggggagaggaaaatnaaaccaaaccaaaattcaaaatctaaccagaccaataatttgggacaattcatttagttt

SEQ ID NO: 233 - DNA, 121 bp, Ocimum basilicum
  variation (61)..(61): at position 61, “n” is either G or A
atggatggaaacggtgccttggactatgaagaatttaaggtacaaaaaattcaatttgttngggaaacagagagggaatcttatggattttgtgttgcggttgcattacagaagcgaatat

SEQ ID NO: 234 - DNA, 121 bp, Ocimum basilicum
  variation (61)..(61): at position 61, “n” is either A or C
tcaatcataactaggaaatcaagtacataatcactcacaacttcagctcattctacaatcntaaaagatattcaatgtcgtgacttcagttcagtgactgatgacagcaaatttctgggta

SEQ ID NO: 235 - DNA, 121 bp, Ocimum basilicum
  variation (61)..(61): at position 61, “n” is either A or G
caattcaattgcagtcatttcttcacagtatataatgatcacgttatctttcgttggatgncttactaacatcattaagtatggaggtcctgaatcaagaatcttgcaaacttttgggtcc

SEQ ID NO: 236 - DNA, 121 bp, Ocimum basilicum
  variation (61)..(61): at position 61, “n” is either C or T
aactcatcacgcaaacaaatactcccactacctctctgcatcggtgttgccccttagatgnggagaagggatgtttctcgcaggagagaaattcaaattctgtgggtacagactagacatg

SEQ ID NO: 237 - DNA, 121 bp, Ocimum basilicum
  variation (61)..(61): at position 61, “n” is either T or A
ggtgttgccccttagatgcggagaagggatgtttctcgcaggagagaaattcaaattctgngggtacagactagacatggcttcctccacatatatatcaaaaacagaacagaagggatag

SEQ ID NO: 238 - DNA, 121 bp, Ocimum basilicum
  variation (61)..(61): at position 61, “n” is either G or A
atcttcaccttggtagctagtactgcaatttgtaaatatctgagcactcatgagcttattntcgaaatattttgtaggcaaaagggttgttgacggaataccaaccaaaatataacagtgc

SEQ ID NO: 239 - DNA, 121 bp, Ocimum basilicum
  variation (61)..(61): at position 61, “n” is either A or G
accttggtagctagtactgcaatttgtaaatatctgagcactcatgagcttattgtcgaantattttgtaggcaaaagggttgttgacggaataccaaccaaaatataacagtgcgagagc

SEQ ID NO: 240 - DNA, 121 bp, Ocimum basilicum
  variation (61)..(61): at position 61, “n” is either C or T
taaatatcgatgagggtggtgtaggtgataacattaggcaaaagtccatcactttgcatcnttcgcaacaactcattcacatcatcccacctctcttgtgccccaaaagaattgatcatga

SEQ ID NO: 241 - DNA, 121 bp, Ocimum basilicum
  variation (61)..(61): at position 61, “n” is either C or G
tcgcagtgtgatggggattttgaaatttcaattctaggactaatgctttccctgtgtatgngtttggtcttaaaaatagccccacaagatccttcttcatcttcctctgctttagttatag

SEQ ID NO: 242 - DNA, 121 bp, Ocimum basilicum
  variation (61)..(61): at position 61, “n” is either T or C
cgcctttcctcgcccattctcagacgccgcagctttcacccggctcttccacctccgccgnctgccgccgccttcattgcacagtgataccaaattatttaaaaacccaggtgaactacaa

SEQ ID NO: 243 - DNA, 121 bp, Ocimum basilicum
  variation (61)..(61): at position 61, “n” is either A or G
ttttctaatgtttaaatttctcccagattattatatgatgaatttcttctccatttgcatncaggcatttcatgtatttcacatttcctgtctcattcaatggatactgctttgtgagagt

SEQ ID NO: 244 - DNA, 121 bp, Ocimum basilicum
  variation (61)..(61): at position 61, “n” is either G or A
aaatttctcccagattattatatgatgaatttcttctccatttgcatacaggcatttcatntatttcacatttcctgtctcattcaatggatactgctttgtgagagtgaaatttttgcaa

SEQ ID NO: 245 - DNA, 121 bp, Ocimum basilicum
  variation (61)..(61): at position 61, “n” is either G or A
aaatccctcacaaacagctgcaagcctctccaatgagattcattagaagaggacaactgancatgcatcaaaattaggaagaagaacatactcctagagcgaggacccctcctatcagtat

SEQ ID NO: 246 - DNA, 121 bp, Ocimum basilicum
  variation (61)..(61): at position 61, “n” is either A or G
gaatggctttgcatgaccagacaactccacatagtcgcgcttctctttgtctttgttctcnctgtgttcagtctctgtagcatcttcttcctggcatttgggttcctctttcactggtttc

SEQ ID NO: 247 - DNA, 121 bp, Ocimum basilicum
  variation (61)..(61): at position 61, “n” is either A or G
tgaaattcagtgtatagaatacaaaatattgcatgcattactgtgtagaggaaattatctnctaagttattgacattcaaactgtctttgccacccatgaggtttataattgccaaagttc

SEQ ID NO: 248 - DNA, 121 bp, Ocimum basilicum
  variation (61)..(61): at position 61, “n” is either G or A
ctgaatggagttctacgggataattggctgaagagtagaacaagaacctggagttggtatnttgatttttgaagtaggaagaacatttcctggctgtactgctccaggagtgaactctagc

SEQ ID NO: 249 - DNA, 121 bp, Ocimum basilicum
  variation (61)..(61): at position 61, “n” is either C or T
tatatatctatataggtttatatttaaataataattcatcttggtgaaatatctaataacngtgcatggttatgtatgagttgtgcagttttgtgttatgaatgttgcatattttggattg

SEQ ID NO: 250 - DNA, 121 bp, Ocimum basilicum
  variation (61)..(61): at position 61, “n” is either A or G
ggtatttggattgttttattctcaccactagtcttgataaaattctttagaacctaaacantatatattctaatgccatgtggcaggtgctggaggaatttcaaaaaccggagaaagacga

SEQ ID NO: 251 - DNA, 121 bp, Ocimum basilicum
  variation (61)..(61): at position 61m “n” is either T or G
ttgttttattctcaccactagtcttgataaaattctttagaacctaaacaatatatattcnaatgccatgtggcaggtgctggaggaatttcaaaaaccggagaaagacgactgcatatct

SEQ ID NO: 252 - DNA, 121 bp, Ocimum basilicum
  variation (61)..(61): at position 61, “n” is either T or C
tactaatactatatccaaaactatcagataaacacgccacgagaaaacaaacttaaactcncaatcacgcctgccgacaaccaaatgcgggacatttgcggtacaatctcttctttgtttg

SEQ ID NO: 253 - DNA, 121 bp, Ocimum basilicum
  variation (61)..(61): at position 61, “n” is either A or G
ggttgccggtggagatggaacagctgggtggcttcttggtgtgatttctgatctcaaattntctcagccgcctccagttgctacagtgcctttaggaacaggaaacaatctaccgtttgca

SEQ ID NO: 254 - DNA, 121 bp, Ocimum basilicum
  variation (61)..(61): at position 61, “n” is either T or A
gagttaaaagattcagattttcttgaccaagttttgctcgcggcttgcagattcaatgacngatttgtagccaaggtatagctgatcgccagatgcctgagccatgaatttgctgctggtt

SEQ ID NO: 255 - DNA, 121 bp, Ocimum basilicum
  variation (61)..(61): at position 61, “n” is either A or G
acacacaaggcacatgcaatccctgcaaaaatcgctccttttcgaacatattttgcagtcncattcgtccacgggaagaagactcctgcagttgagatttctgcatctcatattcagaaat

SEQ ID NO: 256 - DNA, 121 bp, Ocimum basilicum
  variation (61)..(61): at position 61, “n” is either A or G
catctctttaggaaaccggtaaagctttgatttttgtacctgggaaccaaatttagttgcnatatcttttatcctctcgtcaagtgttagtctcatatcaagtgcttcactagagctttta

SEQ ID NO: 257 - DNA, 121 bp, Ocimum basilicum
  variation (61)..(61): at position 61, “n” is either T or A
agtgaagaaacatacaaactcgacccaccttttgccaatctatttcttttccaatttcganccgtacaaccgatgagtgggcgatgatgatggtaatagaaggctaatgcatttgaaaacg

SEQ ID NO: 258 - DNA, 121 bp, Ocimum basilicum
  variation (61)..(61): at position 61, “n” is either G or T
aaacgtttgaaattttcaaatgtttgaaaatttccatcatctcatagcttaatgcaaccgnaataattgtttgtctctcaatggagcaggtatttattgcaatgttggtcgacaatttcct

SEQ ID NO: 259 - DNA, 121 bp, Ocimum basilicum
  variation (61)..(61): at position 61, “n” is either G or T
gaaattttcaaatgtttgaaaatttccatcatctcatagcttaatgcaaccggaataattntttgtctctcaatggagcaggtatttattgcaatgttggtcgacaatttcctttatccta

SEQ ID NO: 260 - DNA, 121 bp, Ocimum basilicum
  variation (61)..(61): at position 61, “n” is either T or C
aattgtttgtctctcaatggagcaggtatttattgcaatgttggtcgacaatttcctttancctatatctgcatttcgatcccatttctctcagtctatcaactggtcaggcataagatat

SEQ ID NO: 261 - DNA, 121 bp, Ocimum basilicum
  variation (61)..(61): at position 61, “n” is either A or G
aagcccttttaaccaaatagcctctttaaaagcttcagttagtgcagtacactcagatttngttgttgataaaacaactatagattgtaactgagacttccaactaacacatgaaccacaa

SEQ ID NO: 262 - DNA, 121 bp, Ocimum basilicum
  variation (61)..(61): at position 61, “n” is either G or A
gcgttccttgacgattgatgtagcatcatcagctcatcaagtatgatttgctgatactganatgatccccttttcatattgggcaggcctttggttgagaacggcaagaatccccagaaat

SEQ ID NO: 263 - DNA, 121 bp, Ocimum basilicum
  variation (61)..(61): at position 61, “n” is either G or C
ttcctgcggtatttatgaaaataaatgattccaagatgtcattttctgtatttccctattnacgacacaagtgcatgtctagtctctatgttctttttattaacttgtcggatctaattat

SEQ ID NO: 264 - DNA, 121 bp, Ocimum basilicum
  variation (61)..(61): at position 61, “n” is either A or G
agcaagtcctatcaggatatgtgaaagcatttagaaatgtatgcaagctccctttcaacanctcaacaaaaggttctttcaaaggatacttccttgaaccacataatacgctgtgctctaa

SEQ ID NO: 265 - DNA, 121 bp, Ocimum basilicum
  variation (61)..(61): at position 61, “n” is either T or C
tggtattttgcactgggttgttgtgcatttaccttacgatgccggcggcggagtatgggangttgaagctgccacgtacgctttccgatctccgctcgctcaagtaagcaactccatctcc

SEQ ID NO: 266 - DNA, 121 bp, Ocimum basilicum
  variation (61)..(61): at position 61, “n” is either C or T
ccatgctcctgaagatgtacctgcatcattggacagaacgttgaaagatttgcagcttgantatgtcgatttgtaccttgtatgcaatttttatcaagtcttttcatcctatactgaagca

SEQ ID NO: 267 - DNA, 121 bp, Ocimum basilicum
  variation (61)..(61): at position 61, “n” is either A or G
tgagattccacaaattagacagtgctttaccaagttggtgaagctgaattaaccacatcancgtacagagttaattaacacgacattgtctaaaaatgtctattgtctccccgttctgtgt

SEQ ID NO: 268 - DNA, 121 bp, Ocimum basilicum
  variation (61)..(61): at position 61, “n” is either G or C
gcccatgaaagggagaggagggagtttgttttgctttttgatggtggagtcgaaagatgcnacgatcttgggggcgtatcagcagaggaatgtgaggatggtgtatgatttgaaagaaggg

SEQ ID NO: 269 - DNA, 121 bp, Ocimum basilicum
  variation (61)..(61): at position 61, “n” is either G or C
gcggagaatccaccgtcgaacaatttatggatcggtaatgtgtcgcaggacgttagtgagnccgacctgaaaactcttttcgagaaatttggcagaatcgacagcgtcacgacctactcat

SEQ ID NO: 270 - DNA, 121 bp, Ocimum basilicum
  variation (61)..(61): at position 61, “n” is either A or T
agctaaatggatatacaataacaaaattaatgaataaaattcctaggttttatagagaaancatactcgagcagagagggaaaaagcaagccgcgatggaggaggcggcagaggagagcct

SEQ ID NO: 271 - DNA, 121 bp, Ocimum basilicum
  variation (61)..(61): at position 61, “n” is either A or T
atattacttttcaaaatgcaaaaaccagcagaagaaaattaatccaaagatctacatccantattacctgtgcagaaggggttttgggctcctttggttctttcccctctttggaggtttt

SEQ ID NO: 272 - DNA, 121 bp, Ocimum basilicum
  variation (61)..(61): at position 61, “n” is either G or A
aggttctagtttaacttataaacacctgaggagcccttgttatatttacttttggtcctanccagttcttgcaccaagagagggaattgtatggtacctacaaaccagatcacaaccatta

SEQ ID NO: 273 - DNA, 121 bp, Ocimum basilicum
  variation (61)..(61): at position 61, “n” is either C or T
tgttcgatgtatccgaaaattccggtcgatgcccctgctcagcgtcgagcaagttctcttncacatatccatcttctggtcggtcattagagttggagaaaagagggaaaccatcatccat

SEQ ID NO: 274 - DNA, 121 bp, Ocimum basilicum
  variation (61)..(61): at position 61, “n” is either A or G
ctccatcgtcttcatccagttctcgaaatctccgatttcctgtcaacatgtacgcatatanatcaaatctcactcattcgagtcgaacactggacaaagattaactcagttttgattcttt

SEQ ID NO: 275 - DNA, 121 bp, Ocimum basilicum
  variation (61)..(61): at position 61, “n” is either G or A
aactattttatcatagactggaactacagaataagcttgtttgacaatttttgcagcctgntaaacagaaaacaggtttaaggagaataagaaaaattatatgaaattattctaaaaatgg

SEQ ID NO: 276 - DNA, 121 bp, Ocimum basilicum
  variation (61)..(61): at position 61, “n” is either C or T
attcctctcccaaccactcaatgacatgagcaaacaatgagcgacttgtaaggggttgtcnaacgacatctgtttccatcaatttccaactaaaatgaaacaattgacaaacaattatatt

SEQ ID NO: 277 - DNA, 121 bp, Ocimum basilicum
  variation (61)..(61): at position 61, “n” is either G or A
cacaaagagaaggtggcagatctgcggaaagcatttcgtgaagaggataaagagtatgatnacgcagaaggagacagcacggaagtggctgatggaaatgatgttggttacttgtcagcag

SEQ ID NO: 278 - DNA, 121 bp, Ocimum basilicum
  variation (61)..(61): at position 61, “n” is either T or G
acagccaatggggccctaattcatctgtacccataattcaagaattattgcattattacanaagacaacaagtggagtgtccgaccatcgttgaatttttttccccctttttttaaaccaa

SEQ ID NO: 279 - DNA, 121 bp, Ocimum basilicum
  variation (61)..(61): at position 61, “n” is either G or C
gtgtatcactttgctgcacttttttgttctccaaggacccttaaaacttaaatatcgatcnatcttgataaaggtgacaaagaattttggatttttttcacgtgcatatcttaaaggaaat

SEQ ID NO: 280 - DNA, 121 bp, Ocimum basilicum
  variation (61)..(61): at position 61, “n” is either G or A
ccagggtttattggaaggcgtcagtgattcattttcaagaaactttcagtttattgactcnatcagagctcatctttcatatacattgttgcgagcatcaatgtcgcagtcttctgtgata

SEQ ID NO: 281 - DNA, 121 bp, Ocimum basilicum
  variation (61)..(61): at position 61, “n” is either T or A
tggtttcagcaattcaaccgcatgtgatttatatgggttttttgtaggtccatatgaaggnggggtttggcaagttcgagtggaacttccagatgcttatccttacaagtctccttcaata

SEQ ID NO: 282 - DNA, 121 bp, Ocimum basilicum
  variation (61)..(61): at position 61, “n” is either T or C
ttctgctattttgttagagtcggtgctgaaagagttggaggcaacaaagaatgaattgacncaagttaaagatgaaagcttccagtttatggcatccatggatgttgttcgagaccaactc

SEQ ID NO: 283 - DNA, 121 bp, Ocimum basilicum
  variation (61)..(61): at position 61, “n” is either G or T
gcatcatgatattgcttctttccatctggcagctgagataaagggtatccaagaactcgantagttttctgcggcccagtatcaaatgaaggatcaaaaatgggattttctgatgattcct

SEQ ID NO: 284 - DNA, 121 bp, Ocimum basilicum
  variation (61)..(61): at position 61, “n” is either G or A
ctgagtctttcaaatattctactgaacgcataacaaagttatttgattatatggctaatangtctcgtcttcgtgacagagagactcataacactctcaagaaagatttgattgagcacat

SEQ ID NO: 285 - DNA, 121 bp, Ocimum basilicum
  variation (61)..(61): at position 61, “n” is either A or G
atcgctgaaacggaccggaatcacagtcgccggcgccgaggaattggtgttcctcggcccntaattgttgaggtatcgttcaattcgatacattagagactgatcactagtgttgattgtt

SEQ ID NO: 286 - DNA, 121 bp, Ocimum basilicum
  variation (61)..(61): at position 61, “n” is either T or C
aaacggaccggaatcacagtcgccggcgccgaggaattggtgttcctcggcccataattgntgaggtatcgttcaattcgatacattagagactgatcactagtgttgattgtttttttgg

SEQ ID NO: 287 - DNA, 121 bp, Ocimum basilicum
  variation (61)..(61): at position 61, “n” is either C or G
cctacaaacacctgcttcaatcacagcttgaattttgtcattcgtaccatcagaaaggtangagagagcccagcatgcatcagtaagtacctcttcatcatcagaatgaaccagacgctca

SEQ ID NO: 288 - DNA, 121 bp, Ocimum basilicum
  variation (61)..(61): at position 61, “n” is either T or C
ttgctacgttccatttggagggggtcatcgcatttgtccaggatacgagttcgcaaagatngaaacacttatcaccattcactatctagttactcgtttcacatggaagctctgctacaac

SEQ ID NO: 289 - DNA, 121 bp, Ocimum basilicum
  variation (61)..(61): at position 61, “n” is either T or C
tgcttgtatggctccattactgttacgtcctcttttagctggtgaatgtgaattagaggangagtatgatattaatggagatagttctgctcagcttctagcggcagccaatgctgctaat

SEQ ID NO: 290 - DNA, 121 bp, Ocimum basilicum
  variation (61)..(61): at position 61, “n” is either G or A
gcttgtatggctccattactgttacgtcctcttttagctggtgaatgtgaattagaggatnagtatgatattaatggagatagttctgctcagcttctagcggcagccaatgctgctaatg

SEQ ID NO: 291 - DNA, 121 bp, Ocimum basilicum
  variation (61)..(61): at position 61, “n” is either A or T
cggcatgatggctggacaattcaaagcatttttcgatgccacgggtagcctatggagaacncagcaactcgctggcaagcccgctgggatcttctacagcactggatctcaaggtggtggc

SEQ ID NO: 292 - DNA, 121 bp, Ocimum basilicum
  variation (61)..(61): at position 61, “n” is either T or C
atattttggtacttcttttggtataatggaattgtaatttgcaggaatgtatgtggcgaanagggcatctgataagatcactcagttaactgataatgtctacatttgccgctctggttcg

SEQ ID NO: 293 - DNA, 121 bp, Ocimum basilicum
  variation (61)..(61): at position 61, “n” is either A or T
aagaggaacgaacaagtcaactcattagaactgagcttgaatcgctgggtattcagtatgngtggcccgtcgctgaaactggcatcgtcgccaccatcggatcgggtgctcgtccatggta

SEQ ID NO: 294 - DNA, 121 bp, Ocimum basilicum
  variation (61)..(61): at position 61, “n” is either G or C
agaggaacgaacaagtcaactcattagaactgagcttgaatcgctgggtattcagtatgantggcccgtcgctgaaactggcatcgtcgccaccatcggatcgggtgctcgtccatggtat

SEQ ID NO: 295 - DNA, 121 bp, Ocimum basilicum
  variation (61)..(61): at position 61, “n” is either C or T
acgaacaagtcaactcattagaactgagcttgaatcgctgggtattcagtatgagtggccngtcgctgaaactggcatcgtcgccaccatcggatcgggtgctcgtccatggtatggtctc

SEQ ID NO: 296 - DNA, 121 bp, Ocimum basilicum
  variation (61)..(61): at position 61, “n” is either C or T
aacaagtcaactcattagaactgagcttgaatcgctgggtattcagtatgagtggcccgtngctgaaactggcatcgtcgccaccatcggatcgggtgctcgtccatggtatggtctcaga

SEQ ID NO: 297 - DNA, 121 bp, Ocimum basilicum
  variation (61)..(61): at position 61, “n” is either C or T
tagagaaatactcttcagagcacttccagggaactggcgaactagaactctgtatccaaanggatttctacaatgtaagaacgaaatataaaatatttaaatcacagactggatattatca

SEQ ID NO: 298 - DNA, 121 bp, Ocimum basilicum
  variation (61)..(61): at position 61, “n” is either A or C
agatggatggtgggcacagcatataaatctcactaagtcagcaacaactgcgtttttctcnggtgtaacaagaaacttctttgtaaaccagacctgatacctcttttggctgccaaattta

SEQ ID NO: 299 - DNA, 121 bp, Ocimum basilicum
  variation (61)..(61): at position 61, “n” is either A or C
ttatacttgtgcatctattatttgtaaattgctcattacaaggtcgatttgattgattgantattccatttgtagaggaaagacattccaagcgagagttttgaaaaggatggttaatggt

SEQ ID NO: 300 - DNA, 121 bp, Ocimum basilicum
  variation (61)..(61): at position 61, “n” is either A or G
tgatttgctcaagtgcatgaattttcactatcatcatgtttaatgttatcgagtcgagatngaggagcttgtacctcaccaactgcttcgagacgaaagttgtccgaacacgaaaccctag

SEQ ID NO: 301 - DNA, 121 bp, Ocimum basilicum
  variation (61)..(61): at position 61, “n” is either G or A
  misc_feature (61)..(61): n is a, c, g, or t
aaagatcaattgctaattcgcgaccaatgattgaggtagaaattgacctggttcccggcgnttggtatggacaactatgattgatgtgaagtctttattatttgcatattcaacaatctga

SEQ ID NO: 302 - DNA, 121 bp, Ocimum basilicum
  variation (61)..(61): at position 61, “n” is either T or C
gtgatttaattttgtctcgtcaaaattgaaagcatatatactacttctggaaagtaaaaantcaaatggtatatcgagatctagattttttcaaacatagactaccataagaactttgaca

SEQ ID NO: 303 - DNA, 121 bp, Ocimum basilicum
  variation (61)..(61): at position 61, “n” is either T or C
tgaacttctggtctatttgtctgtttctctcttatcaatgttatattttgattgccattantcttatttctagttgattttcaagatgtgtattcatgtttgagctctatattactggaat

SEQ ID NO: 304 - DNA, 121 bp, Ocimum basilicum
  variation (61)..(61): at position 61, “n” is either T or C
gctgttgaaagaagaaacacattatcagaatatagtattattttgtctggagatctgcaancaaacttccatcattaatttctacacctcatgaataacaataccacctcatatattaaaa

SEQ ID NO: 305 - DNA, 121 bp, Ocimum basilicum
  variation (61)..(61): at position 61, “n” is either G or T
ctggaaggtttgcaataacagccatccaaggccaaactataggcttgtcatcatctttacnagcagaatgcctcacagctgcatgaaactgggcagctgagccctcttcaggatgaggttt

SEQ ID NO: 306 - DNA, 121 bp, Ocimum basilicum
  variation (61)..(61): at position 61, “n” is either C or A
acaagttgtccaaatatgggagaagcttatttctaaagaattacacaaaattaccctctcnaaggaagatttcttagcttccatgaaaggtaacctatttctcaataagcccccgagatta

SEQ ID NO: 307 - DNA, 121 bp, Ocimum basilicum
  variation (61)..(61): at position 61, “n” is either T or C
ggaagttgttccagggcttgtactccagaagggaacttggaatccaccaacagggatttcnaatgttctcaggttacacttatcgagtccagattacaagatgacattgccaatgcaaccg

SEQ ID NO: 308 - DNA, 121 bp, Ocimum basilicum
  variation (61)..(61): at position 61, “n” is either T or C
agttgttccagggcttgtactccagaagggaacttggaatccaccaacagggatttctaangttctcaggttacacttatcgagtccagattacaagatgacattgccaatgcaaccgagg

SEQ ID NO: 309 - DNA, 121 bp, Ocimum basilicum
  variation (61)..(61): at position 61, “n” is either G or A
aagtaacaacataaatattttatatattatagtacattctaagtactaattaaatattatnttattcatttaatataaatactcacattaaaatatgtgtatagttatagaaataatatat

SEQ ID NO: 310 - DNA, 121 bp, Ocimum basilicum
  variation (61)..(61): at position 61, “n” is either G or T
ttcagattgaagaggcttctaaaaacaccctctgtgcagctatgagcattcacatacagtnttgagatatttgatagcttcacggcactagtgggatccggtaggttgcttcccatgaccc

SEQ ID NO: 311 - DNA, 121 bp, Ocimum basilicum
  variation (61)..(61): at position 61, “n” is either T or C
ttgcaatggcgatcttccattcacgcagtgctttgaatcggcgaggcaggctgggctgctncggtgcagctacaccgaggagcgtgacttgtacgttgccggtgatttgtatgaaaacgat

SEQ ID NO: 312 - DNA, 121 bp, Ocimum basilicum
  variation (61)..(61): at position 61, “n” is either G or T
ggctttttccgtaccttctaactcgacaccgacaaactcaacgtcctcttcaaacaagtanggtgcaccacttctctggctagaattcgacttacctgagccatcgtgggaatcatcggaa

SEQ ID NO: 313 - DNA, 121 bp, Ocimum basilicum
  variation (61)..(61): at position 61, “n” is either A or G
tgaagtagagccatctaaaaactcaccttcaagcatggccatccctcgttgcacgtcttcnggttgtctggaatgaacaagagcccatgacaacctcataatactctcgttaattcgatca

SEQ ID NO: 314 - DNA, 121 bp, Ocimum basilicum
  variation (61)..(61): at position 61, “n” is either T or A
cttctcatgttatatgtatcttattccgtctcttcaatgcttatttgtagagttattcaanatggtatgtttgctctacagggtatagggggtgttggcagtgttgcagctgagatgctaa

SEQ ID NO: 315 - DNA, 121 bp, Ocimum basilicum
  variation (61)..(61): at position 61, “n” is either C or T
gcttatttgtagagttattcaatatggtatgtttgctctacagggtatagggggtgttggnagtgttgcagctgagatgctaacaaggtgtggcatcggtcgcctcttgttgtatgactat

SEQ ID NO: 316 - DNA, 121 bp, Ocimum basilicum
  variation (61)..(61): at position 61, “n” is either G or A
ctttctcattgaaaagatgttcatctatgttatgctgatctatgcatgcaattaatgtggnacaggtgtatctgagaatgctgtttctggtcatatacagttgttggttcctggggaaaca

SEQ ID NO: 317 - DNA, 121 bp, Ocimum basilicum
  variation (61)..(61): at position 61, “n” is either G or A
gcttttgaaattcaatttcgtgaggtcggatgagcgtcgatatcccatccaacggtcatcntgagcagttctagatcaacggcctatattcatagaagtcactaacctcggctcttctccg

SEQ ID NO: 318 - DNA, 121 bp, Ocimum basilicum
  variation (61)..(61): at position 61, “n” is either C or T
tagagctagagacccaatgcatttgtaagatcatcttgattttgaggccgaccgcaatgcngaggcatttcttgaacgccgcccccttcctccgccgctgccatcaccactatttcttctc

SEQ ID NO: 319 - DNA, 121 bp, Ocimum basilicum
  variation (61)..(61): at position 61, “n” is either A or G
ctactagattccctacaatttagaaataatggagctcaaacatatcagacaatgtgctatnttatagttacccccattctgatagtttttgttagtcagttttgtcaattctcattgttcc

SEQ ID NO: 320 - DNA, 121 bp, Ocimum basilicum
  variation (61)..(61): at position 61, “n” is either T or A
atagtatttatcaacaggatcaaatcccacacagcaacatgcaaagctcagaatcaagaanagcaagattgaaaatcttgaaagtccacagcccatcatcatcaaatagaacataaagatg

SEQ ID NO: 321 - DNA, 121 bp, Ocimum basilicum
  variation (61)..(61): at position 61, “n” is either C or T
cttctcacacacatcacactgaatcttcatcttctccgcccaaagatcagatttttaagcnaccccttttcacaatatgaccacccaagaaaaacccatatgccaaaaggccacaaattta

SEQ ID NO: 322 - DNA, 121 bp, Ocimum basilicum
  variation (61)..(61): at position 61, “n” is either C or A
accatctatagaaatgcaggagacatagatcttattgagaatggaaggaaaacgaagaatnacatacgctgttccctgattgtagacatcaatcgagacggagacacgttcagagcccgat

SEQ ID NO: 323 - DNA, 121 bp, Ocimum basilicum
  variation (61)..(61): at position 61, “n” is either T or A
tgtgacaatttttgtaggcatgaccatgttcataggcatgacattccctttctttggtggnttgcttggattctttggtggatttgctttggccccaacaacatactatgtgagtgaccct

SEQ ID NO: 324 - DNA, 121 bp, Ocimum basilicum
  variation (61)..(61): at position 61, “n” is either C or T
tgtaggcatgaccatgttcataggcatgacattccctttctttggtggtttgcttggattntttggtggatttgctttggccccaacaacatactatgtgagtgaccctcttaacatatca

SEQ ID NO: 325 - DNA, 121 bp, Ocimum basilicum
  variation (61)..(61): at position 61, “n” is either G or A
tgaaaattgatgagttaatcttccggattctaaaagaacgcactggagggagccttccaancatatatgattccatacgtgtcatcttatcatccgatgataatcgcgttgtagcctcaca

SEQ ID NO: 326 - DNA, 121 bp, Ocimum basilicum
  variation (61)..(61): at position 61, “n” is either T or G
atactgaacatgatgcctcatctcttcctttaagttgtacaagttgattttggagtgacantggataacgtaaatgggaatcgagaagcagagctcccggagtggaaattatgttggcggc

SEQ ID NO: 327 - DNA, 121 bp, Ocimum basilicum
  variation (61)..(61): at position 61, “n” is either A or G
caaagagtactctcaaaattctttacttgacatgcagagactggcgtccgattttctttcnaattacatctttttggccgttggaagagttggttctagcactggcttaattgttcaaaga

SEQ ID NO: 328 - DNA, 121 bp, Ocimum basilicum
  variation (61)..(61): at position 61, “n” is either G or T
aaggccaagctatccatgtcgaagagtaatagtactacccacctctcaagacatttaaatncatgcttaagacgaaagatattttagaggcagcaaaggaaaatcactttacaaccattag

SEQ ID NO: 329 - DNA, 121 bp, Ocimum basilicum
  variation (61)..(61): at position 61, “n” is either A or G
gggtcgaagcttgggagagtcctgagtgcagagtaaaccgatctttaggaatttgcaggcncgttcggcatcaaaatctccgttgagagctgtgtctacgagcaatacaagctcctttttc

SEQ ID NO: 330 - DNA, 121 bp, Ocimum basilicum
  variation (61)..(61): at position 61, “n” is either A or T
atagttaaatcgagtaaaatcattagtacatgtagcagagaaagttggaagttgttccagnatttacctcacgaagagcatcagcataagaactcatatctgtcaatatcacaagaacgtg

SEQ ID NO: 331 - DNA, 121 bp, Ocimum basilicum
  variation (61)..(61): at position 61, “n” is either T or A
gttctctcactaggattgattgaactccctgaagtaacatgttatagaatgcatcaaatantttggccacttagcatgaatgcacaacattaatttaagattctgcggacacttgccagat

SEQ ID NO: 332 - DNA, 121 bp, Ocimum basilicum
  variation (61)..(61): at position 61, “n” is either A or G
cactattttcgcgttcgcgatggaccagatttgcacagacccatcagcagatcctgcagcnacataaccatcgtcgggactcatacaagatctgctccaattcgacgccactctgcttccg

SEQ ID NO: 333 - DNA, 121 bp, Ocimum basilicum
  variation (61)..(61): at position 61, “n” is either G or A
aaactcttgctgattagattttgattttcttgttgtataaaatgctgtatgaactgcaatncagttatcttgctgagcttctgggctgaagtgcactataattcgatctatccttcgggag

SEQ ID NO: 334 - DNA, 121 bp, Ocimum basilicum
  variation (61)..(61): at position 61, “n” is either A or G
gcaagaaaaaattaaaataattaataatgaaagaaagctagaagtgtgcaatgtggaatgntgagtcactatctaaccaaagtagaccaacaaatagttgataaatcccactagattatca

SEQ ID NO: 335 - DNA, 121 bp, Ocimum basilicum
  variation (61)..(61): at position 61, “n” is either C or T
ttatgcttttcgttccattcaaagaatcgggctctgcacctggctcctcatcattggaggncggaaacatccatcccatgtgatcatcaaccccctccttcgtccttccccatatcggctt

SEQ ID NO: 336 - DNA, 121 bp, Ocimum basilicum
  variation (61)..(61): at position 61, “n” is either T or C
agcagaacctcaagatctcctcaaactcagatataggtgccctgatagcaagaagatctcntgctttttcttcccagcgaacagcactggcatatcttctggagatatcaacaaataactt

SEQ ID NO: 337 - DNA, 121 bp, Ocimum basilicum
  variation (61)..(61): at position 61, “n” is either T or C
gacacactggcacctgatatcgatgatatggacccgtacacaacagagaccgagcattcanaaccgatgcgatcgagtcatgcacctccttgacagcaaacagcacattcttatatatagc

SEQ ID NO: 338 - DNA, 121 bp, Ocimum basilicum
  variation (61)..(61): at position 61, “n” is either C or T
tgtcatcataatggtgaacctgattgccattgcagtcggactaagtcgcacgatttacagngtcataccgcaatggagcaggcttttgggtggcgttttcttcagtttttgggttcttgca

SEQ ID NO: 339 - DNA, 121 bp, Ocimum basilicum
  variation (61)..(61): at position 61, “n” is either A or G
ggtgaacctgattgccattgcagtcggactaagtcgcacgatttacagcgtcataccgcantggagcaggcttttgggtggcgttttcttcagtttttgggttcttgcacatttgtatcca

SEQ ID NO: 340 - DNA, 121 bp, Ocimum basilicum
  variation (61)..(61): at position 61, “n” is either C or A
tattgtggggtaccgagtgtagttaattgggatgtaatcccaacatttgacaagtattgcntaaactatcctctgaggtatttgccaccacgatcagatcgtcgtgacttgatacgttttc

SEQ ID NO: 341 - DNA, 121 bp, Ocimum basilicum
  variation (61)..(61): at position 61, “n” is either A or C
ttcacctgccagcaccaacacgtggcaggagcccagatgcctgtttccatgttaagctaanacactcattattaggagcattgtgacagaaacaagaacaatattaaattggcttcctttt

SEQ ID NO: 342 - DNA, 121 bp, Ocimum basilicum
  variation (61)..(61): at position 61, “n” is either C or A
gcatggccattgtaccgccgcatgtatctgagatacaagaacaaagcttatcaaatcaaantgtaaaacatcacaaaataaggttgcgcaccaatactttctcgaaatattgaagtgtgag

SEQ ID NO: 343 - DNA, 121 bp, Ocimum basilicum
  variation (61)..(61): at position 61, “n” is either T or A
tgttttccgatggcgctggtctggagaccagcagtctactcttctcttgcccaaaaggtcngcttcgtctggtcaagttactattatcctctatattttttactatgtctcaactcctgaa

SEQ ID NO: 344 - DNA, 121 bp, Ocimum basilicum
  variation (61)..(61): at position 61, “n” is either C or T
cacttttctccggagactgctaaagtgatcgatggtgttctgtttcataggaaacaacgcncgattcatctaaacaagttaagctgggatttatcactggacttgttcccagggaaaagtc

SEQ ID NO: 345 - DNA, 121 bp, Ocimum basilicum
  variation (61)..(61): at position 61, “n” is either T or C
gtcatgtttctaccacgtttgcttctgcaattactgctcatttacttcacatttcactttnttattctatattttatttggaccttttgtttttgtggttattagtggtgaatagaaaaag

SEQ ID NO: 346 - DNA, 121 bp, Ocimum basilicum
  variation (61)..(61): at position 61, “n” is either T or A
catctcaagctgttgcttctgtcccgactgaagatgatgatgtgcaaatcattccaacgcnaactgggaagaagagaaagctagccgacgttacggatgctgctgtttcagaagcttcatc

SEQ ID NO: 347 - DNA, 121 bp, Ocimum basilicum
  variation (61)..(61): at position 61, “n” is either C or A
gcatttagtcgaggtgcatcgttaactgaaacataatctacttttgtaaatatttcttgcntgttaaaagggcttatatatgttacctttgtgatgcagaaaccaaccagaggatatgctt

SEQ ID NO: 348 - DNA, 121 bp, Ocimum basilicum
  variation (61)..(61): at position 61, “n” is either T or G
gcttagatctaggtacccaatattttcttggaaaaatacccatataaagaaaacacatttncaagatctgaggcaagaattcatagctaggcaatagtacctttgatatttcatcatatgc

SEQ ID NO: 349 - DNA, 121 bp, Ocimum basilicum
  variation (61)..(61): at position 61, “n” is either C or T
aaaaatacccatataaagaaaacacattttcaagatctgaggcaagaattcatagctaggnaatagtacctttgatatttcatcatatgccatcaagaaaatctgatgattcttatgcatt

SEQ ID NO: 350 - DNA, 121 bp, Ocimum basilicum
  variation (61)..(61): at position 61, “n” is either T or A
atcatatgccatcaagaaaatctgatgattcttatgcatttttatctcattccaatattcnagaaccttctcttcttcggacagcagaccagcaactgtcactattgtaaggggaagacca

SEQ ID NO: 351 - DNA, 121 bp, Ocimum basilicum
  variation (61)..(61): at position 61, “n” is either T or A
ttttgagttatctgtgaagtataaatcaccaatatacccgaccccatccttcaaagtgacngttgtctctccagccactaacggccttctcccttctcgtgtttttactagtttgttgtca

SEQ ID NO: 352 - DNA, 121 bp, Ocimum basilicum
  variation (61)..(61): at position 61, “n” is either T or C
tgagttatctgtgaagtataaatcaccaatatacccgaccccatccttcaaagtgactgtngtctctccagccactaacggccttctcccttctcgtgtttttactagtttgttgtcaaac

SEQ ID NO: 353 - DNA, 121 bp, Ocimum basilicum
  variation (61)..(61): at position 61, “n” is either A or G
cagcagaaccgaaattcttgatttctgaaccgtattcattttcaatctgcattttatgacnagcatgcttgagttcggagttttggattggaaagtttggaacagttgaactgtagaagca

SEQ ID NO: 354 - DNA, 121 bp, Ocimum basilicum
  variation (61)..(61): at position 61, “n” is either T or C
caaactttaatcgatctctttctcggttaggattagggattcggtccttgggagatgatgncttagccgagcctgcttggcgcatgagttctgtaatgcgtgaaaatacaggcttcagaaa

SEQ ID NO: 355 - DNA, 121 bp, Ocimum basilicum
  variation (61)..(61): at position 61, “n” is either G or A
aaagttggtgatggataggatagccaatcggcctagagggtttgcgttcctaagatacaanacagaggaggaatccaagaaagctattgagggaatgcatggaaaggtcagtgccacttct

SEQ ID NO: 356 - DNA, 121 bp, Ocimum basilicum
  variation (61)..(61): at position 61, “n” is either A or T
gttggtgatggataggatagccaatcggcctagagggtttgcgttcctaagatacaagacngaggaggaatccaagaaagctattgagggaatgcatggaaaggtcagtgccacttctcta

SEQ ID NO: 357 - DNA, 121 bp, Ocimum basilicum
  variation (61)..(61): at position 61, “n” is either A or G
acaggcacccatcagtgccccccacaccgccttgtccggcttcaccggcatccactgaatnatctccatcgcctccatgagctctccgtgcctccccactacgtccacaagcgatgagtaa

SEQ ID NO: 358 - DNA, 121 bp, Ocimum basilicum
  variation (61)..(61): at position 61, “n” is either an A or T
catattttatagttgatgtatacaatatgttagtgatgttgaatttgtgtagttgtaatgnttatttttattttcttatcgaacaagattgtggaactatggaccaaaagaaattttaggt

SEQ ID NO: 359 - DNA, 121 bp, Ocimum basilicum
  variation (61)..(61): at position 61, “n” is either C or T
atacaatatgttagtgatgttgaatttgtgtagttgtaatgattatttttattttcttatngaacaagattgtggaactatggaccaaaagaaattttaggtgtgattgtgggaattcaaa

SEQ ID NO: 360 - DNA, 121 bp, Ocimum basilicum
  variation (61)..(61): at position 61, “n” is either G or A
tgatgtaatctttagatacaattcaacatttggccaaaatgttttagtgtcaaaagtaatnttgcaaatggcttctcccttcttcttatttgaatgcacactagttctccactttttagta

SEQ ID NO: 361 - DNA, 121 bp, Ocimum basilicum
  variation (61)..(61): at position 61, “n” is either A or G
cctttcactaattctttcacagagatgtttggacgtttctcttgacttgcaaggtcgcatnctgcagcctctatgataaagggtaaaatttcttttgtaggcctctgagttttgagtcact

SEQ ID NO: 362 - DNA, 121 bp, Ocimum basilicum
  variation (61)..(61): at position 61, “n” is either G or A
ttgtggggcgaacagttcatcaaacaggattccgctgctcatcaggtatatatttataagnttcatttcttacatacattagacatatcatatatgtaattatgtaaatatatatgtgtca

SEQ ID NO: 363 - DNA, 121 bp, Ocimum basilicum
  variation (61)..(61): at position 61, “n” is either G or A
tgctttcaagattttctatttgatttccgattgcattgatatttttgtgctacaaaccatntatattaaattcacatagtatttggggaggctgctagttatattgtttttattcggtctt

SEQ ID NO: 364 - DNA, 121 bp, Ocimum basilicum
  variation (61)..(61): at position 61, “n” is either C or T
ttggaacagtatgtattatttctaagttctctatcatttctttcggagctttagttatagncaataatttactttcctgttgatataatctcaggttagcatgattggcgtacattatgag

SEQ ID NO: 365 - DNA, 121 bp, Ocimum basilicum
  variation (61)..(61): at position 61, “n” is either G or A
attggcgctaaaccagactcacgcgccgggagcagaggggagatggagatcggaaggaatngaaccgccatgaaaatccttcagcgtcatcgtcgccttcttcttcgacattttctcctca

SEQ ID NO: 366 - DNA, 121 bp, Ocimum basilicum
  variation (61)..(61): at position 61, “n” is either A or G
aagtccatagatgaaaaataatgtgggctaatatacttgtagaggggagacattgctgagnaaatcagggagccaggtttagttattaattaattaatctttttcatcttgtagattcagt

SEQ ID NO: 367 - DNA, 121 bp, Ocimum basilicum
  variation (61)..(61): at position 61, “n” is either C or A
catcaccgtgctcaccgtgatcgggtataagagcgaatcggaggccgatcagtgcggcggngtcggaattctcggcattgcatgggccttcggcggcatgattttcatcctggtctactgc

SEQ ID NO: 368 - DNA, 121 bp, Ocimum basilicum
  variation (61)..(61): at position 61, “n” is either C or T
cacatgattaaaaggaagacagataaccaaaatgtctcccgattcatataggtttccagtntacttcttttagcaggggacattgcactgttcagcatagccttagtatcctgcccagcgt

SEQ ID NO: 369 - DNA, 121 bp, Ocimum basilicum
  variation (61)..(61): at position 61, “n” is either G or A
gttaacttggtgatagaacaaagatgcggactatgttaaaaatcagcagcagaatattgcngatgctattgatttgcttcatagactcacaactggcattgatgtgaatataaaattcaga

SEQ ID NO: 370 - DNA, 121 bp, Ocimum basilicum
  variation (61)..(61): at position 61, “n” is either A or G
tcatatgccataatggacagataacatcccaatgcaagtgcaagaaatgaatctgaactcntacaaataaggatgatatagtaggaaaatagaatatataagattctgaacacctcgtata

SEQ ID NO: 371 - DNA, 121 bp, Ocimum basilicum
  variation (61)..(61): at position 61, “n” is either T or C
acacacaaagagtggctcagattcaagtaaataattcatttgttcaaataaatagagaaancaatagacaaggcaattcttttgtcactacctcaataacatagcaacagactgaagctcc

SEQ ID NO: 372 - DNA, 121 bp, Ocimum basilicum
  variation (61)..(61): at position 61, “n” is either G or A
ctgatacaaaaagaactgatgatgcccaggagtaagatcatcatttgtcttgattacatgntgaaggtcagtctccatcaactcaaacacaacatatatatccttgaattctctccgacat

SEQ ID NO: 373 - DNA, 121 bp, Ocimum basilicum
  variation (61)..(61): at position 61, “n” is either A or G
atacaaaaagaactgatgatgcccaggagtaagatcatcatttgtcttgattacatggtgnaggtcagtctccatcaactcaaacacaacatatatatccttgaattctctccgacatgga

SEQ ID NO: 374 - DNA, 121 bp, Ocimum basilicum
  variation (61)..(61): at position 61, “n” is either C or T
gaactgatgatgcccaggagtaagatcatcatttgtcttgattacatggtgaaggtcagtntccatcaactcaaacacaacatatatatccttgaattctctccgacatggaggcaacatg

SEQ ID NO: 375 - DNA, 121 bp, Ocimum basilicum
  variation (61)..(61): at position 61, “n” is either C or A
aaaattcaacaacaaatgaaatcagacagaagaaaagaaatcatcaatatacatgatgctntcatctaccaaacttacctccaaatccacattgattttccacaaggacccattagcgtca

SEQ ID NO: 376 - DNA, 121 bp, Ocimum basilicum
  variation (61)..(61): at position 61, “n” is either C or A
caacaaatgaaatcagacagaagaaaagaaatcatcaatatacatgatgctctcatctacnaaacttacctccaaatccacattgattttccacaaggacccattagcgtcatcaattaca

SEQ ID NO: 377 - DNA, 121 bp, Ocimum basilicum
  variation (61)..(61): at position 61, “n” is either T or C
atgttcaataactaaaacccactttcagttcattgcattagtggttgtctactcagccttnctttttgccttcattgccatcgataatattttttaaacttaacaaagactcaaaagagtg

SEQ ID NO: 378 - DNA, 121 bp, Ocimum basilicum
  variation (61)..(61): at position 61, “n” is either T or C
attcgcgcgaattttcttgaacacgaacatttctcaggttgctagcgcgacagcaactttngtgatgatgttctcctcttctttatccgtggtggagttttacctactcaagagattccca

SEQ ID NO: 379 - DNA, 121 bp, Ocimum basilicum
  variation (61)..(61): at position 61, “n” is either C or T
tctcatcaggtagtcatgattccaacgaggagaatgataaggaggagatccatgacgatcngcctattgaggagcagcaacctgaaatggttgatgaaaaaccatcgattccagcagaaga

SEQ ID NO: 380 - DNA, 121 bp, Ocimum basilicum
  variation (61)..(61): at position 61, “n” is either C or A
caacagttacaacaacagcaaagagaacatgttgctcctactcactttagtccaaaagttnatgcagttaatttgcagcaacctacctctctttcctatggttctacaggagcttcttcct

SEQ ID NO: 381 - DNA, 121 bp, Ocimum basilicum
  variation (61)..(61): at position 61, “n” is either A or G
cccacaccaaccacatcggaggaagcttgattccccatgtgcaactttcttccttcaaccnccttgtaggtggagaaagttcccttgttggaagccacgtgacacgtcatacccgtgtcca

SEQ ID NO: 382 - DNA, 121 bp, Ocimum basilicum
  variation (61)..(61): at position 61, “n” is either T or A
aaatgctattaaactcaagttttaattgcttggtttagagatactaaacgagcttgaacgngcttttatgagttgaaatataagtagcttgctagcaaatatgtccatttatagtcagatg

SEQ ID NO: 383 - DNA, 121 bp, Ocimum basilicum
  variation (61)..(61): at position 61, “n” is either T or C
tattaaactcaagttttaattgcttggtttagagatactaaacgagcttgaacgtgctttnatgagttgaaatataagtagcttgctagcaaatatgtccatttatagtcagatgcttaat

SEQ ID NO: 384 - DNA, 121 bp, Ocimum basilicum
  variation (61)..(61): at position 61, “n” is either A or C
attaaactcaagttttaattgcttggtttagagatactaaacgagcttgaacgtgcttttntgagttgaaatataagtagcttgctagcaaatatgtccatttatagtcagatgcttaatg

SEQ ID NO: 385 - DNA, 121 bp, Ocimum basilicum
  variation (61)..(61): at position 61, “n” is either A or G
ttatctactaatttggatcgacaaatatcatacaattaatatagtattttatacaaataantacttaaaatatataaacattatattatttacaattcaatcttttgaaaacaatgaaagt

SEQ ID NO: 386 - DNA, 121 bp, Ocimum basilicum
  variation (61)..(61): at position 61, “n” is either T or C
ataaacattatattatttacaattcaatcttttgaaaacaatgaaagtttttacgtgcatngcacgtaaacccataaaaatgtaaactacattgatcaaccatagataaaaagatgaaaga

SEQ ID NO: 387 - DNA, 121 bp, Ocimum basilicum
  variation (61)..(61): at position 61, “n” is either A or G
atagtttttcctgatcctcaggggcaatcatgatctgattatatccagaatacccatctanaaagcaataatactcataccttaccagcctatcaagcatctaatcaatgaagggaaggga

SEQ ID NO: 388 - DNA, 121 bp, Ocimum basilicum
  variation (61)..(61): at position 61, “n” is either A or C
ttctttacgtattttgcatgttcacttcacctcaactccaaggctcatctcttgcacagangaaagttctgataaactatctaggagttgtaaatattcgggcagtacttgaaaagcctga

SEQ ID NO: 389 - DNA, 121 bp, Ocimum basilicum
  variation (61)..(61): at position 61, “n” is either A or G
ggcgaaagtgctcatcagattagtcccagctgaggttttcttggaggagaagcatttcttnccattctggctgattaaatccgtggcagatggttcgtgtaatgtcaagaaagaaagtaaa

SEQ ID NO: 390 - DNA, 121 bp, Ocimum basilicum
  variation (61)..(61): at position 61, “n” is either A or G
cgatctctttctagattgcattggcattggtaaatggcagattgtccacaaaatcattccnaaattggtcgcatccagtaattctaccattgatcaagttgatgctatcaaaattttcatt

SEQ ID NO: 391 - DNA, 121 bp, Ocimum basilicum
  variation (61)..(61): at position 61, “n” is either C or T
acaccgtggggtggtctgcttcttctacgccggaatattccacaaaagatacctccttttnttgacaattctctacattatgggcctcatcacctgcgccggaccactgctgagtatcctg

SEQ ID NO: 392 - DNA, 121 bp, Ocimum basilicum
  variation (61)..(61): at position 61, “n” is either G or A
ggaaaaagtagctataaagaaaataacagacatttttgagcatacatctgatgctattcgnattttgcgggagattaagttgcttcgtcttctacgccatcctgatattgttgaaattaag

SEQ ID NO: 393 - DNA, 121 bp, Ocimum basilicum
  variation (61)..(61): at position 61, “n” is either T or C
ttatctagataataagaaagttactatatattctgattctcagtcagctattcaactgtgnaaaaatcttgtgttttatgataggactaaacacattgatgttagatatcattacatttga

SEQ ID NO: 394 - DNA, 121 bp, Ocimum basilicum
  variation (61)..(61): at position 561, “n” is either A or C
aatattttgtacctataaaaacaccatattcagttgaatattgcagaaaacaagacagcangaaattcgaactcactcttttggttcaaatataagttccttgcaaattgcatatgctgca

SEQ ID NO: 395 - DNA, 121 bp, Ocimum basilicum
  variation (61)..(61): at position 61, “n” is either C or T
tgtacctataaaaacaccatattcagttgaatattgcagaaaacaagacagcaagaaattngaactcactcttttggttcaaatataagttccttgcaaattgcatatgctgcagcaccag

SEQ ID NO: 396 - DNA, 121 bp, Ocimum basilicum
  variation (61)..(61): at position 61, “n” is either G or A
gtacctataaaaacaccatattcagttgaatattgcagaaaacaagacagcaagaaattcnaactcactcttttggttcaaatataagttccttgcaaattgcatatgctgcagcaccagc

SEQ ID NO: 397 - DNA, 121 bp, Ocimum basilicum
  variation (61)..(61): at position 61, “n” is either T or C
caccatattcagttgaatattgcagaaaacaagacagcaagaaattcgaactcactctttnggttcaaatataagttccttgcaaattgcatatgctgcagcaccagcaatgccgaggcct

SEQ ID NO: 398 - DNA, 121 bp, Ocimum basilicum
  variation (61)..(61): at position 61, “n” is either A or T
atctacaacatacatatggcatgagaattaatataagaattaacaaaaccatgaacagganatgcaaagtcaagttgaccaccacaaacctgatcgagtgcagaggcaaagatatccagca

SEQ ID NO: 399 - DNA, 121 bp, Ocimum basilicum
  variation (61)..(61): at position 61, “n” is either A or G
tctacaacatacatatggcatgagaattaatataagaattaacaaaaccatgaacaggaantgcaaagtcaagttgaccaccacaaacctgatcgagtgcagaggcaaagatatccagcac

SEQ ID NO: 400 - DNA, 121 bp, Ocimum basilicum
  variation (61)..(61): at position 61, “n” is either T or C
tagctggatgttggtttacgctagtcgcattccaatgggtaagttccttcacttgcttcanattgcccgacattttcgtaatggcataactcttgtttctctggccagtcacggttctcta

SEQ ID NO: 401 - DNA, 121 bp, Ocimum basilicum
  variation (61)..(61): at position 61, “n” is either T or C
gagtttgtattttaatatatcaaatattctaagttcttaatcttgtatatcatatacattncttatgcagtcataatatctaaatgcagctgctcaaaatgctgtaaaacaattcatttag

SEQ ID NO: 402 - DNA, 121 bp, Ocimum basilicum
  variation (61)..(61): at position 61, “n” is either G or A
aaccgcgcccatgatcgagtacggccactcaggagcattgagtttaagcagtctacaaaantagccagcaggagcacggttttttcgatccgtttcagcattcgataccatctcaatccga

SEQ ID NO: 403 - DNA, 121 bp, Ocimum basilicum
  variation (61)..(61): at position 61, “n” is either A or C
gcccatgatcgagtacggccactcaggagcattgagtttaagcagtctacaaaagtagccngcaggagcacggttttttcgatccgtttcagcattcgataccatctcaatccgaccatcg

SEQ ID NO: 404 - DNA, 121 bp, Ocimum basilicum
  variation (61)..(61): at position 61, “n” is either A or G
tgtgtaccttagagaaaagggaatctgcagcttttgtggtacagagccaccgaataaggcngcatcttgagccattctctgcaatattcttctaacaatttcaccaagatacatcccagaa

SEQ ID NO: 405 - DNA, 121 bp, Ocimum basilicum
  variation (61)..(61): at position 61, “n” is either G or T
gattgaaccaacactgcaaaacatacagctttcaaatggttaaagatgtaaaaatctggancatctgatcatatttttcagcaattgtcaataaaattttccaataaattcacataactca

SEQ ID NO: 406 - DNA, 121 bp, Ocimum basilicum
  variation (61)..(61): at position 61, “n” is either C or T
attgaaccaacactgcaaaacatacagctttcaaatggttaaagatgtaaaaatctggagnatctgatcatatttttcagcaattgtcaataaaattttccaataaattcacataactcaa

SEQ ID NO: 407 - DNA, 121 bp, Ocimum basilicum
  variation (61)..(61): at position 61, “n” is either T or C
ctgcaacaaccccggtctgtccttgctcacgcactcaaataatacttgctgacagaattcntggaagtcgagctcaaatctgctgaaaatcacatagaatttctcaagtagaactttagtg

SEQ ID NO: 408 - DNA, 121 bp, Ocimum basilicum
  variation (61)..(61): at position 61, “n” is either C or A
tgcacctgatagaacgatcgacttggggtggcgcatagccatgagagctgcaatgatgaangtttcatctccaatctagcatcatatagagaagatatcagaacaaattaagattactata

SEQ ID NO: 409 - DNA, 121 bp, Ocimum basilicum
  variation (61)..(61): at position 61, “n” is either T or C
cacatttgtgcagttaagttctgccccatgcataaaaaagatggcttaatctagttatttnatggtttttctcatttaccttttaattgtgattgcagtattcagaattgggtcgaaaatt

SEQ ID NO: 410 - DNA, 121 bp, Ocimum basilicum
  variation (61)..(61): at position 61, “n” is either G or A
acgtggtaaagaagtgcatacgcgtattcttgatcccctcgccttctgacgaacttttgcntttctggagacgacgccaccggacttctgaagcttcactacatctacattagctttggtt

SEQ ID NO: 411 - DNA, 121 bp, Ocimum basilicum
  variation (61)..(61): at position 61, “n” is either A or G
aatataagagcagtgcatgaaccaatgttatgcaacacaaattatgtacgctcaataagcnaaacagtacctcatcagccgtataatcagtttcagttataccatccatttcaatcgttgc

SEQ ID NO: 412 - DNA, 121 bp, Ocimum basilicum
  variation (61)..(61): at position 61, “n” is either T or C
atttgtctttccctttaccatcaccaccttacattttctctctttttttttttctattttntcctctctctctcacaatctctctatcttctttatgaaaatctgtcactctcttagcaaa

SEQ ID NO: 413 - DNA, 121 bp, Ocimum basilicum
  variation (61)..(61): at position 61, “n” is either T or C
tttgtctttccctttaccatcaccaccttacattttctctctttttttttttctatttttncctctctctctcacaatctctctatcttctttatgaaaatctgtcactctcttagcaaat

SEQ ID NO: 414 - DNA, 121 bp, Ocimum basilicum
  variation (61)..(61): at position 61, “n” is either C or G
gtgtgtggcctgcatgaatggagctccactgggcggtacagtttcggaaaaggatgacaancgctgttcagatgatttgaggaggaacattggtgcgtatatcaatcttctggtacagaca

SEQ ID NO: 415 - DNA, 121 bp, Ocimum basilicum
  variation (61)..(61): at position 61, “n” is either A or G
actcaatgattaacctaaaagaactgtttgttgcagtggatggaagtccatgctcatcacntttgacccttgactgagactccgcacaactgttttgggaaggtcatccggtgaatatgca

SEQ ID NO: 416 - DNA, 121 bp, Ocimum basilicum
  variation (61)..(61): at position 61, “n” is either G or C
ttctcttccatagggagtgagaagaagtctttggtcacaccatgcacctcgtccaagaaanaatcgtccatcccatgattcaccgcctgattaattaaattataatattatgaaattttat

SEQ ID NO: 417 - DNA, 99 bp, Ocimum basilicum
  variation (61)..(61): at position 61, “n” is either A or G
tatctgtctacatgaagttgtctactcttttgctaattttgccgatgagaagtgaagcacnactttaatcatgagccattttttgtgagattttttagc

SEQ ID NO: 418 - DNA, 121 bp, Ocimum basilicum
  variation (61)..(61): at position 61, “n” is either A or T
cctttcagattttcaacagattttggctggtcagggagccaatgatagtttatctaagatntgctccattcattcatcaaatcttacggaaggttcttgtccagttagtgatgtttccgag

SEQ ID NO: 419 - DNA, 121 bp, Ocimum basilicum
  variation (61)..(61): at position 61, “n” is either T or C
gctggcagtccgcaataaactgaaggatcccctcaagaatctcaactggacgagaaattcngatccttgtgcctcgaattggacaggggtgatatgccaattcaatgagactgatggttat

SEQ ID NO: 420 - DNA, 121 bp, Ocimum basilicum
  variation (61)..(61): at position 61, “n” is either C or T
taagaaatcatcaaaagtattggttgcaggatcccacgccaacaaccttggctaccagtgnggcggttggactattgagtggcagggtcaaagcggtaactctacaattggcaccaccatc

SEQ ID NO: 421 - DNA, 121 bp, Ocimum basilicum
  variation (61)..(61): at position 61, “n” is either C or T
gctcgaggggctggccgacttggaggtggcagtgtgcctgtgcacggccatcaaggccaangtgctcggcatcaatttgaacgtgccggtcgcgcttagcgccttggttagtggttgccag

SEQ ID NO: 422 - DNA, 121 bp, Ocimum basilicum
  variation (61)..(61): at position 61, “n” is either T or C
ttcggatgttagcatatgattcaccagcagatgagtatattaaaaaaggtgagtctactgnaattgaaagcatgaagagattttgtcgagcaattatagagatatttgcagagcaatactt

SEQ ID NO: 423 - DNA, 121 bp, Ocimum basilicum
  variation (61)..(61): at position 61, “n” is either A or G
tttacgacgggcatggaggccggctagctgcggagtatgcccagaagcatttgcacaagantgtgctttcagcaggcttaccacgtgagttggtctgtttatctatgcatttatttggatt

SEQ ID NO: 424 - DNA, 121 bp, Ocimum basilicum
  variation (61)..(61): at position 61, “n” is either C or G
cagaagtaaaaaaatcaaggttatgattctttttgccatttcttataaatggaatacaaanaaaaattacttgtagctcttgttttataaattatttatgttccattttgggctggagtta

SEQ ID NO: 425 - DNA, 121 bp, Ocimum basilicum
  variation (61)..(61): at position 61, “n” is either T or A
ttggatggaggattgccatgccccttttgtttcgggcaagggctttggtgccacgatccgngctatcaacagtatcgaatgtgacgggggtagtgcggccaaggtggcatcgcgtgtaaat

SEQ ID NO: 426 - DNA, 121 bp, Ocimum basilicum
  variation (61)..(61): at position 61, “n” is either A or G
aagctatatctgggaagaggaatgcatgaagacatgctgtcccttctaaagtaagataatnaattcccatcatatcttggaccatatttctctatcaattgtgcatgttaaaaagtaaagt

SEQ ID NO: 427 - DNA, 121 bp, Ocimum basilicum
  variation (61)..(61): at position 61, “n” is either C or G
aatcgtgacacagacagtgacgatgatcagaatcacaaacacgagtaacatgaagccgtanacatagtaaacctgccgatgagaaactggataagtatgttatcaacagcagtggataggc

SEQ ID NO: 428 - DNA, 121 bp, Ocimum basilicum
  variation (61)..(61): at position 61, “n” is either A or T
aataccaagcaacatccgcattgcaagtagagccaataaagatgattccgctgcccacggngtaagaaaggtagccagagaccacagagctaccccccatgccatgaccaccttaccacca

SEQ ID NO: 429 - DNA, 121 bp, Ocimum basilicum
  variation (61)..(61): at position 61, “n” is either G or A
caacatccgcattgcaagtagagccaataaagatgattccgctgcccacggagtaagaaangtagccagagaccacagagctaccccccatgccatgaccaccttaccaccataacgatcc

SEQ ID NO: 430 - DNA, 115 bp, Ocimum basilicum
  variation (55)..(55): at position 55, “n” is either T or C
taataaaaatcaaaatttatataatctataatcaaaatatcaattaaccttgatngaaaaagactaactcttgcacatgtaaattacttgactaaattgtcaattttgagattga

SEQ ID NO: 431 - DNA, 121 bp, Ocimum basilicum
  variation (61)..(61): at position 61, “n” is either A or G
cttgaagaagggctccgtgacctccaaactcttcttcgtcaactttcagcacaatcgcatnatatgcatagataattgcagtagatagcactacaaacctgaatgacaacataaatgtcat

SEQ ID NO: 432 - DNA, 121 bp, Ocimum basilicum
  variation (61)..(61): at position 61, “n” is either G or A
tgctcagcaattttctccaaaacccgtcacctccacttgtgaactcgtagcgaattcctcngtttcgattctcttaaattttcaatattcagatgccggaattctcgaagcttaacgcacg

SEQ ID NO: 433 - DNA, 121 bp, Ocimum basilicum
  variation (61)..(61): at position 61, “n” is either G or A
gatcttttccagctttctgatgtccctgtgcagaagactgtttatgttataagcaaagcantagagaagttatctggttgtaagtttcagatttttgcattggtctacactagaatttctt

SEQ ID NO: 434 - DNA, 121 bp, Ocimum basilicum
  variation (61)..(61): at position 61, “n” is either T or C
tttcccaacatcagggcaccagatgaccagacaaatttgagttgccaagatccctatcaanacggtagcaaaatgataagtttaagaaacacaagcttcacaaagcaatgagaaaaattat

SEQ ID NO: 435 - DNA, 121 bp, Ocimum basilicum
  variation (61)..(61): at position 61, “n” is either C or T
gggggcattctcgtacatttcgtttgggggtggaaggcatggctgtttgggggagccgttngcttatcttcagatcaaagtgatatggagccatttactgagaaacttcgagatggaactt

SEQ ID NO: 436 - DNA, 121 bp, Ocimum basilicum
  variation (61)..(61): at position 61, “n” is either A or T
catgtgattctgatcctagtttggcttgtagaaaaatactacatatctagatatgttgganctaattgcaatttctgatgtgaaagtgggtggtaattctgaaacatgtaatcttattgaa

SEQ ID NO: 437 - DNA, 121 bp, Ocimum basilicum
  variation (61)..(61): at position 61, “n” is either T or C
tgcgtgtagttgttagatgcatctaagaaaaactggaggtttggtttgtaatactagtaantccatcttgcagtagtggaatcttttctatttgaatatccagcaacagaggaaccataat

SEQ ID NO: 438 - DNA, 121 bp, Ocimum basilicum
  variation (61)..(61): at position 61, “n” is either G or T
gatgcatctaagaaaaactggaggtttggtttgtaatactagtaattccatcttgcagtantggaatcttttctatttgaatatccagcaacagaggaaccataattttggaactcataat

SEQ ID NO: 439 - DNA, 121 bp, Ocimum basilicum
  variation (61)..(61): at position 61, “n” is either A or T
aacgtcttccatcagatgccttgtatgaagaacgagcaattttgttgggaaagatgaaccnacacgagcttgctctctcaatatacgttcacaaggtacttcatcacatagtactttggtt

SEQ ID NO: 440 - DNA, 121 bp, Ocimum basilicum
  variation (61)..(61): at position 61, “n” is either G or A
atcaattttctaactagtctctttctctctttctcgtaaacagaggcatatttatggccangagggacaaaacatcgatatatttgcagataatagtagtttatccataaacccttcttat

SEQ ID NO: 441 - DNA, 121 bp, Ocimum basilicum
  variation (61)..(61): at position 61, “n” is either T or A
aactatttataagttataattggtaaaggtaggttggaagtgtaggttccatagaaattgnaatagggtgttttggatgtgtggtgagtccatattcttcttccgtggatatatcttcacc

SEQ ID NO: 442 - DNA, 121 bp, Ocimum basilicum
  variation (61)..(61): at position 61, “n” is either Tor C
aattacatctgttatggatgattgcatgatcacggtcctcgaatactccttccatggcctncctaggtacgtagggaagtcaccttgaacagacttcaagtccgaggtagctccaatccta

SEQ ID NO: 443 - DNA, 121 bp, Ocimum basilicum
  variation (61)..(61): at position 61, “n” is either G or A
cgtgggtcttagatggagttatttggatggttgagatttagttcatagttcactcaccagnattagttcgtatttgaacacaaaccctattacataactttatttgttaagttatgaaaat

SEQ ID NO: 444 - DNA, 121 bp, Ocimum basilicum
  variation (61)..(61): at position 61, “n” is either C or T
tcgctttttgagggaacaccgatcggagtgggcagattttaacgttgacgcctttgctgcnatgtccttgaagtcgagctcatgcacgtatcccggaactaggcctacaagattcactgga

SEQ ID NO: 445 - DNA, 121 bp, Ocimum basilicum
  variation (61)..(61): at position 61, “n” is either G or A
ggaacaccgatcggagtgggcagattttaacgttgacgcctttgctgccatgtccttgaantcgagctcatgcacgtatcccggaactaggcctacaagattcactggaacccaaataatc

SEQ ID NO: 446 - DNA, 121 bp, Ocimum basilicum
  variation (61)..(61): at position 61, “n” is either T or C
cgtaaaatacttttttctctttaaaaaatcaacatgaagtccaaactccaaattgaaagantattttattttcacaaaattcccgcaaatttaacattttgttactcatggatattaaaat

SEQ ID NO: 447 - DNA, 121 bp, Ocimum basilicum
  variation (61)..(61): at position 61, “n” is either G or A
catttttggaaaatgttatgaagaggtggtttttgtgtggagatgagacgagagagagagncttagcagttttggaagtgaagaaggggggcggagttgatgaggagaaggggttgggatt

SEQ ID NO: 448 - DNA, 121 bp, Ocimum basilicum
  variation (61)..(61): at position 61, “n” is either C or T
atttttggaaaatgttatgaagaggtggtttttgtgtggagatgagacgagagagagaggnttagcagttttggaagtgaagaaggggggcggagttgatgaggagaaggggttgggattg

SEQ ID NO: 449 - DNA, 121 bp, Ocimum basilicum
  variation (61)..(61): at position 61, “n” is either A or G
tttggaaaatgttatgaagaggtggtttttgtgtggagatgagacgagagagagaggcttngcagttttggaagtgaagaaggggggcggagttgatgaggagaaggggttgggattggag

SEQ ID NO: 450 - DNA, 121 bp, Ocimum basilicum
  variation (61)..(61): at position 61, “n” is either A or G
catcggcttctgcggtctccctcttctccttgcaggtagtttctcatcctcaccgcttccngcatcatcacgagcagcaattgcagattgtttcttccctttccctctccctctacccatt

SEQ ID NO: 451 - DNA, 120 bp, Ocimum basilicum
  variation (61)..(61): at position 61, “n” is either C or T
ggcgtggggcgaactggcggtggaatttgggagagggtttagggatgttgtgcagcagacnatattgaccgaagattcttatattgtggagaaaaccaagggaccgttggccgaggtatc

SEQ ID NO: 452 - DNA, 121 bp, Ocimum basilicum
  variation (61)..(61): at position 61, “n” is either T or A
gggcagtgaccaaataatttgcatcaattctattggttctccactatcattgagagatacnctactcaaatctgtggactatactcaaaatgggacactcactgtttatttcttgcaatta

SEQ ID NO: 453 - DNA, 121 bp, Ocimum basilicum
  variation (61)..(61): at position 61, “n” is either A or T
tttgcatcaattctattggttctccactatcattgagagatactctactcaaatctgtggnctatactcaaaatgggacactcactgtttatttcttgcaattaatgtagccatgtgtagc

SEQ ID NO: 454 - DNA, 121 bp, Ocimum basilicum
  variation (61)..(61): at position 61, “n” is either A or G
caggactcaaaaaatgatcttcttttcaatttaggacccaaaagatcgatttttgatccangacctgaaagatgtcaaaattttcaatttaagactcgaaatatgtcaaacttttcaatcc

SEQ ID NO: 455 - DNA, 121 bp, Ocimum basilicum
  variation (61)..(61): at position 61, “n” is either C or T
gactcctcccccaatgtcccatcctgaaaaggcagaacatcaaaactaatgcatgtataanagagaattatagtcaaatcagatcagacacaatccaaaagaacttttggagtataacacg

SEQ ID NO: 456 - DNA, 121 bp, Ocimum basilicum
  variation (61)..(61): at position 61, “n” is either T or C
gtcgggagggaagatttttatcgatggggttgacatatcggctttggggctccatgatctnaggtctaggtttgggatcattcctcaagagcctgtgctctttgaggggacggtgaggagc

SEQ ID NO: 457 - DNA, 121 bp, Ocimum basilicum
  variation (61)..(61): at position 61, “n” is either A or C
tcgggagggaagatttttatcgatggggttgacatatcggctttggggctccatgatcttnggtctaggtttgggatcattcctcaagagcctgtgctctttgaggggacggtgaggagca

SEQ ID NO: 458 - DNA, 121 bp, Ocimum basilicum
  variation (61)..(61): at position 61, “n” is either T or G
agggaagatttttatcgatggggttgacatatcggctttggggctccatgatcttaggtcnaggtttgggatcattcctcaagagcctgtgctctttgaggggacggtgaggagcaatatc

SEQ ID NO: 459 - DNA, 121 bp, Ocimum basilicum
  variation (61)..(61): at position 61, “n” is either T or C
gcttcattccttatctgtccaatgagacattaaagaaaaaaaactaaaagatacttctganaacttgggtgctttaaagttttaaacagaaaaactaataaatactaacttgtgtctctgg

SEQ ID NO: 460 - DNA, 121 bp, Ocimum basilicum
  variation (61)..(61): at position 61, “n” is either C or T
gttgtaagcatgcataagtttcctctctgcagagctcaactgaaggtcgtcaacattcacnactcgccctaagatctttaatctttccctgaatggtacggtcatgtcaatggcaaacccc

SEQ ID NO: 461 - DNA, 121 bp, Ocimum basilicum
  variation (61)..(61): at position 61, “n” is either C or T
gctcttgaatcagctctgcaatcttaagatggaaaacactgaatcgcagctgcaaaacganagcgccgccgtcagaacacaaccgccgccgccgcctttcgccgtcgacctccttgaattg

SEQ ID NO: 462 - DNA, 121 bp, Ocimum basilicum
  variation (61)..(61): at position 61, “n” is either G or A
ttgaatcagctctgcaatcttaagatggaaaacactgaatcgcagctgcaaaacgacagcnccgccgtcagaacacaaccgccgccgccgcctttcgccgtcgacctccttgaattgcaga

SEQ ID NO: 463 - DNA, 121 bp, Ocimum basilicum
  variation (61)..(61): at position 61, “n” is either C or G
gagcgaagtcttcaaggctgggaaggcagacattgatcaaaatgaggccatgaacaaagtnatgagtaaatttgacaacaaccaggacgacaaaatcagtgagcaagaattcatcgaagga

SEQ ID NO: 464 - DNA, 121 bp, Ocimum basilicum
  variation (61)..(61): at position 61, “n” is either G or A
aattttgcagtttgccatgcattactcatggtctacggaggcacgtgccaggcgagccttngccaatgtatcagctttacttcggccaggaggcatcttcattggaacaatgcctgatgcc

SEQ ID NO: 465 - DNA, 121 bp, Ocimum basilicum
  variation (61)..(61): at position 61, “n” is either A or T
caacaaatattccaacattgtccgaaacagcacaattaatctaacatgttgttttgatganttttgaattaattaggttggaggatacgccgttgagtaccaaaacgtgacatttgtcacg

SEQ ID NO: 466 - DNA, 121 bp, Ocimum basilicum
  variation (61)..(61): at position 61, “n” is either G or A
ccccagccttaaagtctcacttataacctgataaaatcataatttattaaatttattattnttgaagcttgaaaagaaggattctatttatagggaggggaggaaaattcttacgcattga

SEQ ID NO: 467 - DNA, 121 bp, Ocimum basilicum
  variation (61)..(61): at position 61, “n” is either A or G
gtactttgtaatggggaacaaaagcgtgacgctagtctgatccaaaagcttgtcctggtcncacagcattcaatttcataaatcaacccgtaaaagtaagctaagaaacaaagaatcagag

SEQ ID NO: 468 - DNA, 121 bp, Ocimum basilicum
  variation (61)..(61): at position 61, “n” is either C or G
gaaattcaatacaggaatattgttttcccgatgtggaatttcaatttttgcaacgtttttnaatctcaatttgcgttttcaataccgattagaattgggattagggtttctaattttgcgc

SEQ ID NO: 469 - DNA, 121 bp, Ocimum basilicum
  variation (61)..(61): at position 61, “n” is either T or C
attgttttcccgatgtggaatttcaatttttgcaacgtttttcaatctcaatttgcgtttncaataccgattagaattgggattagggtttctaattttgcgcttttgacgacagaaatgg

SEQ ID NO: 470 - DNA, 121 bp, Ocimum basilicum
  variation (61)..(61): at position 61, “n” is either T or G
aactgtaaatacctttatgtccctgtgcattattttattcttgtgcagatactctaacccnaccaacaactgttttgtgtacattctgataacctgaaatgcaccatgaaatttatgatgt

SEQ ID NO: 471 - DNA, 121 bp, Ocimum basilicum
  variation (61)..(61): at position 61, “n” is either T or C
tggcaagtggaatcataaaacgagatgtgtgattctgaggtcaatcaatcacctgtcttcngccctcattttgtcaaattcatcgatgcaaacaactcctccatctgccaggaccatggca

SEQ ID NO: 472 - DNA, 121 bp, Ocimum basilicum
  variation (61)..(61): at position 61, “n” is either T or G
atcataaaacgagatgtgtgattctgaggtcaatcaatcacctgtcttctgccctcatttngtcaaattcatcgatgcaaacaactcctccatctgccaggaccatggcacctccttccag

SEQ ID NO: 473 - DNA, 121 bp, Ocimum basilicum
  variation (61)..(61): at position 61, “n” is either T or C
caatcacctgtcttctgccctcattttgtcaaattcatcgatgcaaacaactcctccatcngccaggaccatggcacctccttccagataaaactctcgctgtattaacagcaaagcatgc

SEQ ID NO: 474 - DNA, 121 bp, Ocimum basilicum
  variation (61)..(61): at position 61, “n” is either G or T
ttttgcaggggcggatcccttgatgaacaagacgagggacatttcggtaattggcggaacnggcgatttcttcatgagcagaggagtggccacattgatgacggatgcttttgaaggagag

SEQ ID NO: 475 - DNA, 121 bp, Ocimum basilicum
  variation (61)..(61): at position 61, “n” is either C or G
tgcaggggcggatcccttgatgaacaagacgagggacatttcggtaattggcggaacgggngatttcttcatgagcagaggagtggccacattgatgacggatgcttttgaaggagaggtt

SEQ ID NO: 476 - DNA, 121 bp, Ocimum basilicum
  variation (61)..(61): at position 61, “n” is either T or C
atagtaaaaatacttaacaagaggcatccatctttcttcaatgctacatgccatcagttgnggtttgtgtgctaatagtcttcccatatcttcatcgctaagcccaaactccttcagatat

SEQ ID NO: 477 - DNA, 121 bp, Ocimum basilicum
  variation (61)..(61): at position 61, “n” is either A or G
aggaagcagacaaagtgaagctctttataagatatcatggaaatttgccgacaccccacanccaaaagccaagtaagagtaaaattatgtgtagggggtctgcctttattctgtccccatg

SEQ ID NO: 478 - DNA, 121 bp, Ocimum basilicum
  variation (61)..(61): at position 61, “n” is either G or A
cacacatctttctcagaaggttcccctcattccaagaaaagaaggatctaacgttagaccnaaaatcacgacactcgacaaagccattagagagttagagaagatagttgcagaatgtatt

SEQ ID NO: 479 - DNA, 121 bp, Ocimum basilicum
  variation (61)..(61): at position 61, “n” is either C or A
aatcacgacactcgacaaagccattagagagttagagaagatagttgcagaatgtatttanaaattctactgccgtggtgtttttctttctcttatgtgatattttgggctatgacttact

SEQ ID NO: 480 - DNA, 121 bp, Ocimum basilicum
  variation (61)..(61): at position 61, “n” is either A or G
atcattcattataatcacattaggagatgatgcattttctcattgcacaggcagaaaaagngggatcaatcctctcaaaattattcaccattctgataaaaaggtgaaaaagctaaagcta

SEQ ID NO: 481 - DNA, 121 bp, Ocimum basilicum
  variation (61)..(61): at position 61, “n” is either A or C
ggagatgatgcattttctcattgcacaggcagaaaaagagggatcaatcctctcaaaattnttcaccattctgataaaaaggtgaaaaagctaaagctatacaagaatatgcaaattgggt

SEQ ID NO: 482 - DNA, 121 bp, Ocimum basilicum
  variation (61)..(61): at position 61, “n” is either T or C
aaattattcaccattctgataaaaaggtgaaaaagctaaagctatacaagaatatgcaaantgggtgtcaaatctttcctgaatattcaacttttcatttaaatgttcttttaagaaaaat

SEQ ID NO: 483 - DNA, 121 bp, Ocimum basilicum
  variation (61)..(61): at position 61, “n” is either T or A
tccacttttctgccttttccatgtaaagtaatgcaaatcagcaaaagtttagacaacaatntaacagagtattgacagcaaaaagtacaaaataaaacagtattcatttcatcccactcca

SEQ ID NO: 484 - DNA, 121 bp, Ocimum basilicum
  variation (61)..(61): at position 61, “n” is either G or C
ccaataagagaaatgacatcgagatagtccgtttctgtagtcagctgatgaattaaatctntcgttacatcttcgaagcccaccacatctttctgccttggtggccggacctgttccaaaa

SEQ ID NO: 485 - DNA, 121 bp, Ocimum basilicum
  variation (61)..(61): at position 61, “n” is either T or C
tccaagatagattctttccgcaagaataaaggagcccattattgctacgatgatcatcatnaaagggctaaaagcagtggcaaacactggtcctttgctcttcatcacaagtccttggata

SEQ ID NO: 486 - DNA, 121 bp, Ocimum basilicum
  variation (61)..(61): at position 61, “n” is either C or T
tgccggataatggaagcgcacctacgtggtacgccatctctgctcttctctatcgtgaacngtaaaaccaacagtgctggtagctttaaaccacaacttgtgactttgatagatttgctgt

SEQ ID NO: 487 - DNA, 121 bp, Ocimum basilicum
  variation (61)..(61): at position 61, “n” is either C or T
aattgtaacagtaggaaaattcatttgtggctaattaataaactatcaaaaatcctgcacntattgttgggacttgggctttgtattttattgacagcgactcgtgcatttgtatgaagaa

SEQ ID NO: 488 - DNA, 121 bp, Ocimum basilicum
  variation (61)..(61): at position 61, “n” is either G or A
tcaattgaggaagagaaaagccagattgaggaattaaaccttgttagcgtctttggggacnccataaccgcaataatacatctgacctacgagcacctgcatgttgatgtcgccagttttg

SEQ ID NO: 489 - DNA, 121 bp, Ocimum basilicum
  variation (61)..(61): at position 61, “n” is either G or A
atgaactcttaatatagctagaagaaatcttcttaagatataagtccccgtaattgataancataccttgccattcgagaattggacaattctgcgagaggtaggtagaaaatgagatatc

SEQ ID NO: 490 - DNA, 121 bp, Ocimum basilicum
  variation (61)..(61): at position 61, “n” is either C or T
catgaagtataccattgtaaacgagaacattgccaaattcatcagtcaaaggttgcaaaancggattcactcctcgaagttgcagcgttgccccaataaataacagttccccacggaattc

SEQ ID NO: 491 - DNA, 121 bp, Ocimum basilicum
  variation (61)..(61): at position 61, “n” is either C or A
tattttcattattgcaatcatagtaatattatgatttacaaacacatttcaaatttttttnttttatcatattcttattggaacttttaagagaatattataataatacacgacgttcgtt

SEQ ID NO: 492 - DNA, 121 bp, Ocimum basilicum
  variation (61)..(61): at position 61, “n” is either T or A
aatattatgcaaatataaaataaaaggttgaaaaaaaaatacttacttttcaagaaaatangcaaaggggcagaaaagaagggtggcagcaacatggcgatagacgaccaaaatccaatga

SEQ ID NO: 493 - DNA, 121 bp, Ocimum basilicum
  variation (61)..(61): at position 61, “n” is either G or T
ctttaccaaaacactaacacgaaattaaccttccaatctccaaatactgacagaatcaacngaagaaggatccagattttcggaaaaatcagctcgtcatattcacaacaaactccaaatc

SEQ ID NO: 494 - DNA, 121 bp, Ocimum basilicum
  variation (61)..(61): at position 61, “n” is either G or C
aaacactaacacgaaattaaccttccaatctccaaatactgacagaatcaacggaagaagnatccagattttcggaaaaatcagctcgtcatattcacaacaaactccaaatcaaatcaaa

SEQ ID NO: 495 - DNA, 121 bp, Ocimum basilicum
  variation (61)..(61): at position 61, “n” is either C or T
ctccaaatactgacagaatcaacggaagaaggatccagattttcggaaaaatcagctcgtnatattcacaacaaactccaaatcaaatcaaatcaaatccgaaggccgaaagtagagaacc

SEQ ID NO: 496 - DNA, 121 bp, Ocimum basilicum
  variation (61)..(61): at position 61, “n” is either A or G
tttgacgataatcttgtgaggataaacaaagaggcttgactcggagaggacatatgagtcnaaatgttcattcgacagcgaagcagctatggtgcattcggcaggttgcagaaattcatcg

SEQ ID NO: 497 - DNA, 121 bp, Ocimum basilicum
  variation (61)..(61): at position 61, “n” is either C or T
caagctgaatgagttgatgattcaggggaacaaaaatggagtcgaaggtctaaggatgatngatgctcaagaagctacgcggctagaaccagagctacactgcacgaaagccttgctttcg

SEQ ID NO: 498 - DNA, 121 bp, Ocimum basilicum
  variation (61)..(61): at position 61, “n” is either C or A
gaatatgtctttttggaagagattttgactgtagaaggccacgtcgagtaagaaccatgtngaagtcgtgccgaggaggtggccgccgtggcgccggaagaattctttcgagaagagggtg

SEQ ID NO: 499 - DNA, 121 bp, Ocimum basilicum
  variation (61)..(61): at position 61, “n” is either C or T
tttcatgacgttatagttatgttgtttgcctagatcaacggccattatcttttgtttatangcaggcagcagctcgtggtttaccaattgtcgccacgaaaaatggaggtccggttgacat

SEQ ID NO: 500 - DNA, 121 bp, Ocimum basilicum
  variation (61)..(61): at position 61, “n” is either A or G
gtttaaaataaccttcaacaagtttccacgtgagaaaagctcaggcctcctaattgggagnatgacagtttcatacagtgccttcttcacatcttcaagggcaccaacgtcgtcaaacttc

SEQ ID NO: 501 - DNA, 121 bp, Ocimum basilicum
  variation (61)..(61): at position 61, “n” is either T or A
ttggttccagcaccaattaaacccaacgaaatgatagctgcctgtcacacccagacagtangcatattaagagaataattgccaaaataagaatcgggaaagaaaaatgaccaagcctgtt

SEQ ID NO: 502 - DNA, 121 bp, Ocimum basilicum
  variation (61)..(61): at position 61, “n” is either A or G
accggccatgaatcatctgccaagaactatgaatctgcgggtgacgaggttcctacagggntgttcgacagcaggctcgatagcaggaaaagaaaactgagcttccatcaagatcactcta

SEQ ID NO: 503 - DNA, 121 bp, Ocimum basilicum
  variation (61)..(61): at position 61, “n” is either A or G
gttgattcatgtatatccaccgctttttttaacacacataatccgataataaaatctgacntaaaatgacggactgattaatctatacgtacgtctgcacaataaagcgtgctttccaact

SEQ ID NO: 504 - DNA, 121 bp, Ocimum basilicum
  variation (61)..(61): at position 61, “n” is either T or G
ttgattcatgtatatccaccgctttttttaacacacataatccgataataaaatctgacanaaaatgacggactgattaatctatacgtacgtctgcacaataaagcgtgctttccaactc

SEQ ID NO: 505 - DNA, 121 bp, Ocimum basilicum
  variation (61)..(61): at position 61, “n” is either A or C
gattcatgtatatccaccgctttttttaacacacataatccgataataaaatctgacatanaatgacggactgattaatctatacgtacgtctgcacaataaagcgtgctttccaactcta

SEQ ID NO: 506 - DNA, 121 bp, Ocimum basilicum
  variation (61)..(61): at position 61, “n” is either A or G
acaacttacgcaattcattaacaagattatatttttacgttttaatttgttattagatatnttatttgataaatgagttgatgaggtgtttgggcattttacctttcacgataggtataat

SEQ ID NO: 507 - DNA, 121 bp, Ocimum basilicum
  variation (61)..(61): at position 61, “n” is either A or C
accagttgtagtctggcaagcccgcgatgggtcccaccgcactaatcgcaacatcctgcanggcagcatattaatacagggaattagagcctatgaaaacacagtagaaactcgagccatc

SEQ ID NO: 508 - DNA, 121 bp, Ocimum basilicum
  variation (61)..(61): at position 61, “n” is either G or A
atattgattagcaaagacacaggttggtgtttataagttgttgcacatgaagttgctattnagtttttcttgatcctatttctgatgtgattgggctgaatgaggaatatacattgcaggt

SEQ ID NO: 509 - DNA, 121 bp, Ocimum basilicum
  variation (61)..(61): at position 61, “n” is either A or G
cgaggccttgctccactgcatccgctagcccgtcaatatatcctagaaaaacaaaaatcgnaaactttctaacacgcgtcgtgaagttaagaaaaccgtatgcaacgatagtacttaagag

SEQ ID NO: 510 - DNA, 121 bp, Ocimum basilicum
  variation (61)..(61): at position 61, “n” is either G or A
gcatccgctagcccgtcaatatatcctagaaaaacaaaaatcgaaaactttctaacacgcntcgtgaagttaagaaaaccgtatgcaacgatagtacttaagagaaaccttcatttcccca

SEQ ID NO: 511 - DNA, 121 bp, Ocimum basilicum
  variation (61)..(61): at position 61, “n” is either G or T
tcaggctatagtttttcttattgtctattccttaataatggctataattgaagctgccttnttttcttttataacagctggcaggcgttgtgaatgtgggatgtccagtaggctgtatgct

SEQ ID NO: 512 - DNA, 121 bp, Ocimum basilicum
  variation (61)..(61): at position 61, “n” is either C or T
caaagtccccgtctggttagtttgctcccaccaactcctcactatgtccctgttcactttngacttctcgattgacacaacatagttcaactttactccgagtctatggagagcaacttct

SEQ ID NO: 513 - DNA, 121 bp, Ocimum basilicum
  variation (61)..(61): at position 61, “n” is either A or G
gctgcttcctcttctgcgtatataacatgactatttccatcaagctgcaacaatcacaccnaccaatatccagggttaataagttatgaataggatcaatatctggcacgcaataacgaaa

SEQ ID NO: 514 - DNA, 121 bp, Ocimum basilicum
  variation (61)..(61): at position 61, “n” is either C or A
tcttctgcgtatataacatgactatttccatcaagctgcaacaatcacaccaaccaatatncagggttaataagttatgaataggatcaatatctggcacgcaataacgaaacaatcataa

SEQ ID NO: 515 - DNA, 121 bp, Ocimum basilicum
  variation (61)..(61): at position 61, “n” is either T or C
gacagcaccgccaacatcatcgagcagtagctcctttctctttgctctatttgatattggnccatttttagcaaatgcttgatcagattgatgaattgaagtatcaaaagcatctgatgta

SEQ ID NO: 516 - DNA, 121 bp, Ocimum basilicum
  variation (61)..(61): at position 61, “n” is either A or G
attggcagcttctagaagctcctcacggtcatcttctgactcccctgaaaattacaataanttaagcctcatctaaattttcctttgcagtaaaggaaaggacaaactgtatccaaaaaat

SEQ ID NO: 517 - DNA, 121 bp, Ocimum basilicum
  variation (61)..(61): at position 61, “n” is either A or C
caccttcaactcagaatccccaaatcctaagagaaagctacactaccaaaccctaacaaanggaataattaccaaatcatcatgtacacggccttcccctcaacgcaactaattctaccca

SEQ ID NO: 518 - DNA, 121 bp, Ocimum basilicum
  variation (61)..(61): at position 61, “n” is either A or T
ttcaactcagaatccccaaatcctaagagaaagctacactaccaaaccctaacaaaaggantaattaccaaatcatcatgtacacggccttcccctcaacgcaactaattctacccaaacc

SEQ ID NO: 519 - DNA, 121 bp, Ocimum basilicum
  variation (61)..(61): at position 61, “n” is either T or A
tcaactcagaatccccaaatcctaagagaaagctacactaccaaaccctaacaaaaggaanaattaccaaatcatcatgtacacggccttcccctcaacgcaactaattctacccaaaccc

SEQ ID NO: 520 - DNA, 121 bp, Ocimum basilicum
  variation (61)..(61): at position 61, “n” is either G or A
atgatcgttcacctgtttatgatgagtcaaggggcctacaaaatgattcttttgctcgaanggacagttcagggcctcgtgtgaaagaagcttctcggaaagatgaattagagtccgacag

SEQ ID NO: 521 - DNA, 121 bp, Ocimum basilicum
  variation (61)..(61): at position 61, “n” is either A or T
gaagcttttgtggggcaacaaaaagaacactgctactgaagaggtatttaaaaacatttcnccattacttcttagttctgcttttgcaatttacttttatatcactattacttctttgttg

SEQ ID NO: 522 - DNA, 121 bp, Ocimum basilicum
  variation (61)..(61): at position 61, “n” is either C or T
ccccgacgtggaaaagcagcgagagcaacttcaggcagacctcgagaagcaatatactgtngggctacgtcgaagagatggccgtacagttggactcggtctttaggactaatccttaggt

SEQ ID NO: 523 - DNA, 121 bp, Ocimum basilicum
  variation (61)..(61): at position 61, “n” is either A or G
tgggagactgggaggagaggtccaatgtgggaaggaaggttacagagtgtgtaagaatccngatgaatatttgttttgggatttctttcatccctctgaaaggacttacaagctcatctcc

SEQ ID NO: 524 - DNA, 121 bp, Ocimum basilicum
  variation (61)..(61): at position 61, “n” is either A or G
agttgcctgatagcaattctgtcccatgcaaatatcctccctttttgatgaaggatgagantttcttgctctgttgtagactactgctgcaaaatctccaaacaagaatgaacctagtggt

SEQ ID NO: 525 - DNA, 121 bp, Ocimum basilicum
  variation (61)..(61): at position 61, “n” is either C or T
gtcccatgcaaatatcctccctttttgatgaaggatgagaatttcttgctctgttgtagantactgctgcaaaatctccaaacaagaatgaacctagtggtatattagacactacaatatt

SEQ ID NO: 526 - DNA, 121 bp, Ocimum basilicum
  variation (61)..(61): at position 61, “n” is either C or G
tctcccaaaatgattctaccatttttttccaaattagaagtgagcaagttttgccgaagantatagctcgctgctcgtgatgtaatttaaggtaattttttttattctgattgttgctgag

SEQ ID NO: 527 - DNA, 121 bp, Ocimum basilicum
  variation (61)..(61): at position 61, “n” is either A or G
tcccaaaatgattctaccatttttttccaaattagaagtgagcaagttttgccgaagactntagctcgctgctcgtgatgtaatttaaggtaattttttttattctgattgttgctgagtg

SEQ ID NO: 528 - DNA, 121 bp, Ocimum basilicum
  variation (61)..(61): at position 61, “n” is either T or C
cttcggtgaagttgagttgtttgatggttcccggctcgccattgccttggaggaattcgangctcttgatggaagaaaacatgagttgaggggcaagattgtgagaatccaagatcaaggc

SEQ ID NO: 529 - DNA, 121 bp, Ocimum basilicum
  variation (61)..(61): at position 61, “n” is either A or C
gttgtttgatggttcccggctcgccattgccttggaggaattcgatgctcttgatggaagnaaacatgagttgaggggcaagattgtgagaatccaagatcaaggcattgaacatcctatt

SEQ ID NO: 530 - DNA, 121 bp, Ocimum basilicum
  variation (61)..(61): at position 61, “n” is either A or G
ttgtttgatggttcccggctcgccattgccttggaggaattcgatgctcttgatggaaganaacatgagttgaggggcaagattgtgagaatccaagatcaaggcattgaacatcctattt

SEQ ID NO: 531 - DNA, 121 bp, Ocimum basilicum
  variation (61)..(61): at position 61, “n” is either C or T
caaggcattgatcaagattgaatgttttgaaggttgctatacctccattgagccacaagancagaggtttggtggaagaattggaaggagactcgacaaaataataaaacagtgctcttcc

SEQ ID NO: 532 - DNA, 121 bp, Ocimum basilicum
  variation (61)..(61): at position 61, “n” is either G or A
tagacggggtaaattaccaagtgcaaaatggcaaggaagtgacttagcaagcttccgcaanttggtcatctcagcattggttagtcgtggacgcattccagtaggaagaagcctaaacggc

SEQ ID NO: 533 - DNA, 121 bp, Ocimum basilicum
  variation (61)..(61): at position 61, “n” is either C or A
gaatgaactgtaaaattgatgatggtttgcatcaacatatcaacatcatagaacttctgcnaacagagagggggtattaagacatacccacttcaaaggcccatcaccattgtcagtttta

SEQ ID NO: 534 - DNA, 121 bp, Ocimum basilicum
  variation (61)..(61): at position 61, “n” is either C or A
tccggagcagatctgcttcctgcgatttatctcgaattttcattgcacagttggtgatagnacgttcatactctgcattttcttcctgcaacatagcctctctctgctgtgcctgcattga

SEQ ID NO: 535 - DNA, 121 bp, Ocimum basilicum
  variation (61)..(61): at position 61, “n” is either A or G
atcctttaaaccaccaaagtgagaatctacttctttaaacccacaacgggatattctcatncgcagaaactcttattgtgaactattcatttcacattggattttaatgcagcttaatgtt

SEQ ID NO: 536 - DNA, 121 bp, Ocimum basilicum
  variation (61)..(61): at position 61, “n” is either C or T
tttaggttgattgctcgcgtggggaaaatgggagaaccttaatcacttgtgtatttccgantgggagagcagaataggaatgattacgaggggataatcgctgctgcgaatggcagtgtga

SEQ ID NO: 537 - DNA, 121 bp, Ocimum basilicum
  variation (61)..(61): at position 61, “n” is either T or C
tgcgaacttgtgaattaagatgttctccattttcttgtattgatgatatttcactggctancaggccgacaatcttttcttaacacagcaaagtggatcgaggaggttcgcactgagagag

SEQ ID NO: 538 - DNA, 121 bp, Ocimum basilicum
  variation (61)..(61): at position 61, “n” is either G or C
ccgccgcggcgcaccgtagcaccgaccaattcgcatcggtgttctcgagttcgaaacggcngaatttcagctccagcgtgaggtgcgcgttgctcccgcagaccaggcttgaaagcagacg

SEQ ID NO: 539 - DNA, 121 bp, Ocimum basilicum
  variation (61)..(61): at position 61, “n” is either A or T
ttttacttgcaggaccttttgagaggtacttctgatgtttttcctctattagattaacaangattgtgttacagtagagttcccaaaggataaaatataaagagaagcagagacaataaag

SEQ ID NO: 540 - DNA, 121 bp, Ocimum basilicum
  variation (61)..(61): at position 61, “n” is either A or C
gaactccccctaagaattgatttcggagagcaagcacttatttccaaattttcagaagcancggctaccgaaacttcaggctctagttcttcagctatcacttctggaagcttaagcaata

SEQ ID NO: 541 - DNA, 121 bp, Ocimum basilicum
  variation (61)..(61): at position 61, “n” is either A or G
ctgaacagcgcctttcattccctttgcagggtccgtgagcaccaactcagcaccaaaagcncggagaataattctgcgttcaagactcattgaagcgggcatggttataatgagcttgtag

SEQ ID NO: 542 - DNA, 121 bp, Ocimum basilicum
  variation (61)..(61): at position 61, “n” is either A or T
gatggagcccagaggttttatacattcaagattgagccaaggtttacaatgctggcagganggagctcaaaagttttaagggcctcgagctcagttcttttgcagtcccaagagatctttg

SEQ ID NO: 543 - DNA, 121 bp, Ocimum basilicum
  variation (61)..(61): at position 61, “n” is either C or T
aggcgaagtagatctcaaatacgaattggaacatctaatcatactgactgcaagtagatgnttgttgggtgaagaggttcgcaacaagctcttcgatgatgtctctgctctcttccatcat

SEQ ID NO: 544 - DNA, 121 bp, Ocimum basilicum
  variation (61)..(61): at position 61, “n” is either C or G
gtctcctccaaaagttcttgccagaccaaagtctgatatctttgggttcatgttggaatcnagtaatatattgctagctttgaggtcccggtggataactcggaggcgggaatcttgatgg

SEQ ID NO: 545 - DNA, 121 bp, Ocimum basilicum
  variation (61)..(61): at position 61, “n” is either A or G
tcctatttcttcaataatttcgactgtggaaccatctgcagtggctaatagcaagcacccnttcatgagaaatttcatactgccagttcctgatgcctcatgcccagcagtactgataaaa

SEQ ID NO: 546 - DNA, 121 bp, Ocimum basilicum
  variation (61)..(61): at position 61, “n” is either T or C
aggtgtcttcatggatttcagctacgtggctcctgaatatgcgaatacgggcttgctgaangaaaaaagcgacgtctacagcttcggtgttctgcttctggaatgtgttactggaagagat

SEQ ID NO: 547 - DNA, 121 bp, Ocimum basilicum
  variation (61)..(61): at position 61, “n” is either A or T
agaagagaagaatgagcttgggttgggagttgtgggaagaactagctagacaatgttgtanagatgatgaatttgtgggtgagcttatatctgaagttgttggtcctgtttgattttgcgg

SEQ ID NO: 548 - DNA, 121 bp, Ocimum basilicum
  variation (61)..(61): at position 61, “n” is either C or G
agctattctttcctcatacatttgggttgtattagcaagttttctctgggatgccgagtcntctagttgcttttgctactaacaaagaagcaaatggcattaatttagttgtcaaaaattt

SEQ ID NO: 549 - DNA, 121 bp, Ocimum basilicum
  variation (61)..(61): at position 61, “n” is either A or G
atctctaggatttgatctgacttctactaatttttggatgttatagccagtcttctgtggntaaactgattggtgctccgcagaacctgaatctgttggttcaaaattgtatcaactgcaa

SEQ ID NO: 550 - DNA, 121 bp, Ocimum basilicum
  variation (61)..(61): at position 61, “n” is either G or T
tgtcttgattcattcacaggtttataggcatcaagattgtggaaacattattccttctccnatgtgggacaagaaatgacttgtgagctcgagttcgagctcgaactcgaacatgttagcg

SEQ ID NO: 551 - DNA, 121 bp, Ocimum basilicum
  variation (61)..(61): at position 61, “n” is either A or G
cgagtgtaattgttggccttggtggaagaatctgccatcggagcaagcattcaagaaagcncagaaggagattgaattcggagaagcattatcatcgtggagaagctgaagaaatttgcag

SEQ ID NO: 552 - DNA, 121 bp, Ocimum basilicum
  variation (61)..(61): at position 61, “n” is either T or G
gaattatgctcacaactcccaatttcctgccaatggcaatgtgtctaatttcggccagagnattggagtcttgaaccagaatacggatgtgtatatcggtggaagatcgagtggaagcgct

SEQ ID NO: 553 - DNA, 121 bp, Ocimum basilicum
  variation (61)..(61): at position 61, “n” is either G or A
caactcccaatttcctgccaatggcaatgtgtctaatttcggccagagtattggagtcttnaaccagaatacggatgtgtatatcggtggaagatcgagtggaagcgctttagctcctatg

SEQ ID NO: 554 - DNA, 121 bp, Ocimum basilicum
  variation (61)..(61): at position 61, “n” is either G or A
ttgattatatatttaactaataccactttagaagcattaagcagaaaatcacagtacctcntctagtgcagtcccagaaaaaattgatccaattgccttcatcagtgcaggaaacatcact

SEQ ID NO: 555 - DNA, 121 bp, Ocimum basilicum
  variation (61)..(61): at position 61, “n” is either C or G
agagccggagagaatcgtcttcccggaaagcagttgcaggattataacaccgtaagcatanacgtcactcttctcggtgaaacggccggtggtgatgtattccggcgccatgtaacccaag

SEQ ID NO: 556 - DNA, 121 bp, Ocimum basilicum
  variation (61)..(61): at position 61, “n” is either G or A
gccggagagaatcgtcttcccggaaagcagttgcaggattataacaccgtaagcatacacntcactcttctcggtgaaacggccggtggtgatgtattccggcgccatgtaacccaaggcg

SEQ ID NO: 557 - DNA, 121 bp, Ocimum basilicum
  variation (61)..(61): at position 61, “n” is either G or A
aaactcttgaagcctcttttggaactctgataatcctcaacttctccctaatttgaaaacnttaagagaaaccttaagtttcatcagttttcatagttaatgttgtcaattaaaagtgatg

SEQ ID NO: 558 - DNA, 121 bp, Ocimum basilicum
  variation (61)..(61): at position 61, “n” is either C or T
gaagatcaagcacaagaagaagaaggtcaagctcgccgtgctccagttctacaaggtcgangattctggcaaggtccagcgcctgcgcaaggagtgccccaacgccgactgtggcgccgga

SEQ ID NO: 559 - DNA, 121 bp, Ocimum basilicum
  variation (61)..(61): at position 61, “n” is either A or C
actgtaacaaaaaattaaccaaccaataacgtgtgactaaaatagatactctctctatatnctgtgacaaaactaattcacacaaagaaaatctgtccaacgttcaaaaggctttcatgag

SEQ ID NO: 560 - DNA, 121 bp, Ocimum basilicum
  variation (61)..(61): at position 61, “n” is either A or T
ccctagttggaagggaatcaaggtagaaaaaagttcagttttaattcactacgatgatctnaaaactaatgctgctttgtcttgttatttcaggtgatcctttctgatgctactgtccctg

SEQ ID NO: 561 - DNA, 121 bp, Ocimum basilicum
  variation (61)..(61): at position 61, “n” is either A or T
tcccgccaccggcggagtgaagaaaccccaccgcttccgtccaggaacagttgctctccgngagattaggaagtaccagaaatctactgagctgcttatccgtaaacttccgttccagagg

SEQ ID NO: 562 - DNA, 121 bp, Ocimum basilicum
  variation (61)..(61): at position 61, “n” is either T or C
tgagtccaacagaaatcaattgtagcttttgaactttttttctggtttgttttttaaaagnaatctgctttgcttctttcttgttactctctcattattgtgttgcctaattattgaaaat

SEQ ID NO: 563 - DNA, 121 bp, Ocimum basilicum
  variation (61)..(61): at position 61, “n” is either C or A
gtgttctatcaatcttattaccaaaatgcaaacatacaaaattataatttttctagcaacnattctgtctctctcaatagcaaatgcagagacacaatatcatcaatttgttgtatgcctc

SEQ ID NO: 564 - DNA, 121 bp, Ocimum basilicum
  variation (61)..(61): at position 61, “n” is either C or T
aatcttattaccaaaatgcaaacatacaaaattataatttttctagcaaccattctgtctntctcaatagcaaatgcagagacacaatatcatcaatttgttgtatgcctccattaacctt

SEQ ID NO: 565 - DNA, 121 bp, Ocimum basilicum
  variation (61)..(61): at position 61, “n” is either T or A
cttattaccaaaatgcaaacatacaaaattataatttttctagcaaccattctgtctctcncaatagcaaatgcagagacacaatatcatcaatttgttgtatgcctccattaaccttccc

SEQ ID NO: 566 - DNA, 121 bp, Ocimum basilicum
  variation (61)..(61): at position 61, “n” is either C or T
catacaaaattataatttttctagcaaccattctgtctctctcaatagcaaatgcagaganacaatatcatcaatttgttgtatgcctccattaaccttcccttagttttaattctcacag

SEQ ID NO: 567 - DNA, 121 bp, Ocimum basilicum
  variation (61)..(61): at position 61, “n” is either C or A
ggtattagggaacagattgtgctgtgaaatctctagtttaagagattgagcatcgtccaanagccctaagtcgcaaaatcccttgattaacacattataacattgagtgtcgggaactacc

SEQ ID NO: 568 - DNA, 121 bp, Ocimum basilicum
  variation (61)..(61): at position 61, “n” is either T or G
gaagagtacagctaagaaataatcgaccaaatcggtgccggcttcttggaagacgtcgatnataggagtggagtttttctgagcgagccaacccataacgccccattttgcagccatttct

SEQ ID NO: 569 - DNA, 121 bp, Ocimum basilicum
  variation (61)..(61): at position 61, “n” is either G or A
gaatgttctagtatcgagaagtcgtaccggtttttgaaccattccaagtcgtctcgaccgnaacgtagcaacagtagcagcaacatccagagcagtcatatccccatcaagaactctttcg

SEQ ID NO: 570 - DNA, 121 bp, Ocimum basilicum
  variation (61)..(61): at position 61, “n” is either T or A
gtgctcttatgctctatttcttgtcaatcaccttaagactataacatagttccgctgatantattttgctttgtggctcagatgatctacctttcgagttcagggcattggaagttgcttt

SEQ ID NO: 571 - DNA, 121 bp, Ocimum basilicum
  variation (61)..(61): at position 61, “n” is either A or G
gctcttatgctctatttcttgtcaatcaccttaagactataacatagttccgctgatattnttttgctttgtggctcagatgatctacctttcgagttcagggcattggaagttgctttgg

SEQ ID NO: 572 - DNA, 121 bp, Ocimum basilicum
  variation (61)..(61): at position 61, “n” is either C or T
atttagatattggagaattgctttttcacgccagcgccaccatcaaattcaagatttaaantccgacttggggccatattatattctgatatttatattcgaaatgttaaactagcacctt

SEQ ID NO: 573 - DNA, 121 bp, Ocimum basilicum
  variation (61)..(61): at position 61, “n” is either A or G
cgtcatccggctgcccaatcagcttcagaatcggcgtcgcgaagatgaagatcgggagcancaccacgcagcagatgaatagcacgatccacgatcgctgcagatatattcctaacatgtg

SEQ ID NO: 574 - DNA, 121 bp, Ocimum basilicum
  variation (61)..(61): at position 61, “n” is either C or G
ctaatttataaagctgtaatatgcaaattgtccattttgatgatgatattaatggaatganggtgttaatagtggtgatgtactctttaagcatgcataattccatctataattataatct

SEQ ID NO: 575 - DNA, 121 bp, Ocimum basilicum
  variation (61)..(61): at position 61, “n” is either A or G
tgtttggtagataaagtaaatgaaagggcattcgacataaaatgatataccgagatccagnagacacaaaaatcaagcattcatttcacctcattgatttccttttctatccactcctgat

SEQ ID NO: 576 - DNA, 121 bp, Ocimum basilicum
  variation (61)..(61): at position 61, “n” is either T or C
tgaaagggcattcgacataaaatgatataccgagatccagaagacacaaaaatcaagcatncatttcacctcattgatttccttttctatccactcctgatttttgtaagtgaaatcaagt

SEQ ID NO: 577 - DNA, 121 bp, Ocimum basilicum
  variation (61)..(61): at position 61, “n” is either G or A
gtaggagatattgggattgcatgaatgctgtttcggagattccatgtctggcatgaacganacgtgatcgtcgtaccaaacgggcgaatcgtgctcgctcatcttctgcgttgtggctatt

SEQ ID NO: 578 - DNA, 121 bp, Ocimum basilicum
  variation (61)..(61): at position 61, “n” is either G or A
gggattgcatgaatgctgtttcggagattccatgtctggcatgaacgagacgtgatcgtcntaccaaacgggcgaatcgtgctcgctcatcttctgcgttgtggctattcttttcttgaaa

SEQ ID NO: 579 - DNA, 121 bp, Ocimum basilicum
  variation (61)..(61): at position 61, “n” is either T or A
agcagtaaaataatgttgcatttgtacattaaattttaatttggtagcactgaaagtgtgntacctttctttagtgttcgactgagcatatcagattgttctgaagtttctggttttgatg

SEQ ID NO: 580 - DNA, 121 bp, Ocimum basilicum
  variation (61)..(61): at position 61, “n” is either A or G
tattcaatcattgagcaatgatttttcgtcagacaattcctccttcgtatcatcggcatcntcatgggaatgggaatgggaatgggaatggaattccctccccttcaatgaaaacgattcc

SEQ ID NO: 581 - DNA, 121 bp, Ocimum basilicum
  variation (61)..(61): at position 61, “n” is either T or G
attcaatcattgagcaatgatttttcgtcagacaattcctccttcgtatcatcggcatcancatgggaatgggaatgggaatgggaatggaattccctccccttcaatgaaaacgattccc

SEQ ID NO: 582 - DNA, 121 bp, Ocimum basilicum
  variation (61)..(61): at position 61, “n” is either G or A
gtggtattgttctgtttctttatgttgctgtatgtttttctggacttgtacttagtgactncatttttctaacacagaccttggagagatccgtccaattcttccacaattgcttgatggt

SEQ ID NO: 583 - DNA, 121 bp, Ocimum basilicum
  variation (61)..(61): at position 61, “n” is either T or G
tttccaaagggaaccaagctgatccttgtcaaaggaaaagcaatggtgaggatgcttatanatgaggcgtaagttattcttaagagaaactagtgatgcgtaagctgaagccatggatctc

SEQ ID NO: 584 - DNA, 121 bp, Ocimum basilicum
  variation (61)..(61): at position 61, “n” is either A or G
aagctgatccttgtcaaaggaaaagcaatggtgaggatgcttatatatgaggcgtaagttnttcttaagagaaactagtgatgcgtaagctgaagccatggatctcttctaaatttctttc

SEQ ID NO: 585 - DNA, 121 bp, Ocimum basilicum
  variation (61)..(61): at position 61, “n” is either C or G
tgattcaagtagtaacaattcaaccctctctctctgaagcaaaatcagcaaagtgaaaaanagtgtaaattcaatgagggagaagctgctgtcaaaataacaaattaaaaaacagagcata

SEQ ID NO: 586 - DNA, 121 bp, Ocimum basilicum
  variation (61)..(61): at position 61, “n” is either G or C
attcaagtagtaacaattcaaccctctctctctgaagcaaaatcagcaaagtgaaaaacantgtaaattcaatgagggagaagctgctgtcaaaataacaaattaaaaaacagagcataat

SEQ ID NO: 587 - DNA, 121 bp, Ocimum basilicum
  variation (61)..(61): at position 61, “n” is either C or A
ggtttaaatataagaaaaggacagttcaggttccagtcttccagacttgataaatgaatgncaaaactaaaggcagaaaaggtaacagtaacatgagtgtattaataagagaactgagaag

SEQ ID NO: 588 - DNA, 121 bp, Ocimum basilicum
  variation (61)..(61): at position 61, “n” is either G or T
acagttcaggttccagtcttccagacttgataaatgaatgccaaaactaaaggcagaaaangtaacagtaacatgagtgtattaataagagaactgagaaggaccgtacaccaaaaatctc

SEQ ID NO: 589 - DNA, 121 bp, Ocimum basilicum
  variation (61)..(61): at position 61, “n” is either T or C
cttccagacttgataaatgaatgccaaaactaaaggcagaaaaggtaacagtaacatgagngtattaataagagaactgagaaggaccgtacaccaaaaatctcccaatgatatttatatt

SEQ ID NO: 590 - DNA, 121 bp, Ocimum basilicum
  variation (61)..(61): at position 61, “n” is either G or A
actggtgagcgcgtaaagatagatggaatatagaaagtaaatgttggttgaaatacctgtngatttgaagcattacttttgggactctttcctcggtacaaaacccaaaaagtgaagaaaa

SEQ ID NO: 591 - DNA, 121 bp, Ocimum basilicum
  variation (61)..(61): at position 61, “n” is either C or G
aaaagtttggtatatattaatgagagtcacatattataatattctactgcataaagctcantttgtgtgcatgagcaagttgacaacttggaatcgtgcactatgtacaaaccttaaaata

SEQ ID NO: 592 - DNA, 121 bp, Ocimum basilicum
  variation (61)..(61): at position 61, “n” is either G or A
tatattaatgagagtcacatattataatattctactgcataaagctcactttgtgtgcatnagcaagttgacaacttggaatcgtgcactatgtacaaaccttaaaatattgtcctagctg

SEQ ID NO: 593 - DNA, 121 bp, Ocimum basilicum
  variation (61)..(61): at position 61, “n” is either A or G
tactatttacatggcatccgaatgcagttgcaaagacgggaatcgttgtgaagagactgancccggaaacacctccagcgaagtcaggaaacagtctcggtgtactcgttttcccactcca

SEQ ID NO: 594 - DNA, 121 bp, Ocimum basilicum
  variation (61)..(61): at position 61, “n” is either A or G
tccgaatgcagttgcaaagacgggaatcgttgtgaagagactgaacccggaaacacctccngcgaagtcaggaaacagtctcggtgtactcgttttcccactccacagtgcgtgtatcgcc

SEQ ID NO: 595 - DNA, 121 bp, Ocimum basilicum
  variation (61)..(61): at position 61, “n” is either G or C
ttctgaagcaacacagccacctaagagatggactgaagtgctcctataccttgacatgaanagaatgcctcctagtttcttagcaattctatgcaaagacttgattttctgtggcagcaag

SEQ ID NO: 596 - DNA, 121 bp, Ocimum basilicum
  variation (61)..(61): at position 61, “n” is either A or C
accattcattcttatatggattttcatcaaagaaattggtttcaaataccacaacaaacancatcaatcctcaatttctactctctggaaactaagaagattgtttgtcttgaaaattcag

SEQ ID NO: 597 - DNA, 121 bp, Ocimum basilicum
  variation (61)..(61): at position 61, “n” is either T or C
gaattaattttggtggaaaactctgctggtaagtacagcattcatcaatgaacaggatatnttcgttacttcaaatttttcttcacttttaattggtttttacaggtttctgtcacctggg

SEQ ID NO: 598 - DNA, 121 bp, Ocimum basilicum
  variation (61)..(61): at position 61, “n” is either A or T
aagaaccaggtcagaagattttgtttcaagtttttgtgattacaaacaaacaaagctgcangatgctaacctgttggaaagtgctgagagacgccaagatggagcctccgatccagacact

SEQ ID NO: 599 - DNA, 121 bp, Ocimum basilicum
  variation (61)..(61): at position 61, “n” is either G or C
agaaccaggtcagaagattttgtttcaagtttttgtgattacaaacaaacaaagctgcaanatgctaacctgttggaaagtgctgagagacgccaagatggagcctccgatccagacactg

SEQ ID NO: 600 - DNA, 121 bp, Ocimum basilicum
  variation (61)..(61): at position 61, “n” is either G or A
accaggtcagaagattttgtttcaagtttttgtgattacaaacaaacaaagctgcaagatnctaacctgttggaaagtgctgagagacgccaagatggagcctccgatccagacactgtat

SEQ ID NO: 601 - DNA, 121 bp, Ocimum basilicum
  variation (61)..(61): at position 61, “n” is either G or T
taatcttgtaccatcctgataaataaacgtgacacgagatttgacaattttgtaatgattntgtccttagatatgcactgcagtgggagtcaaagcatctatatgctgtaagcactgcaat

SEQ ID NO: 602 - DNA, 121 bp, Ocimum basilicum
  variation (61)..(61): at position 61, “n” is either T or C
cccctcccggcatagattgtgtagagctgtgtttagggtcaccgtatccattttcaaaccnttcccacccatttgatgcatcattttaaaagcctttgcaaaatcaccagatttaaaaaat

SEQ ID NO: 603 - DNA, 121 bp, Ocimum basilicum
  variation (61)..(61): at position 61, “n” is either C or A
cttgtcaaaaggggacgatctagccatgactttgatcttgtctactctctccattctctcntcttctgtgtagttgcgaaactcggagccttctactactgatccatcatttggatcttgg

SEQ ID NO: 604 - DNA, 121 bp, Ocimum basilicum
  variation (61)..(61): at position 61, “n” is either C or T
taagtttgataagagtttgaagaagttaattactcttgcagcgttgatttgggcctgaatnatttgcggcacgtaacctcgtacttcttcgatggatttgccttggactaacgcatatccc

SEQ ID NO: 605 - DNA, 121 bp, Ocimum basilicum
  variation (61)..(61): at position 61, “n” is either A or G
tttgaagaagttaattactcttgcagcgttgatttgggcctgaatcatttgcggcacgtancctcgtacttcttcgatggatttgccttggactaacgcatatcccagatcatttgcttcc

SEQ ID NO: 606 - DNA, 121 bp, Ocimum basilicum
  variation (61)..(61): at position 61, “n” is either T or C
gaagttaattactcttgcagcgttgatttgggcctgaatcatttgcggcacgtaacctcgnacttcttcgatggatttgccttggactaacgcatatcccagatcatttgcttccgagaat

SEQ ID NO: 607 - DNA, 121 bp, Ocimum basilicum
  variation (61)..(61): at position 61, “n” is either C or T
aaactcacgttaatacaagagccaatgtctgtttgcccgagggtagtttgtcccgatcagntgaaagtgtgctcagtttagcatcaacaggccgatatgggattcttacctgcaaatttta

SEQ ID NO: 608 - DNA, 121 bp, Ocimum basilicum
  variation (61)..(61): at position 61, “n” is either  A or G
tagccggccgtcaattcttcagcttttagctaaaagcttggtcgggtgtgaatcggctggngaagctcgaactttgatgtgtccatgtttgtactgtacattagtgaccttcagcttgtac

SEQ ID NO: 609 - DNA, 121 bp, Ocimum basilicum
  variation (61)..(61): at position 61, “n” is either G or C
ataattaaatcctaacctcatggccttaatcataattaaatgacccttcatcgactttttntaattgaacaacatttcttaatcatgtttagtgtgtttggttgttcaaaaagatggagtc

SEQ ID NO: 610 - DNA, 121 bp, Ocimum basilicum
  variation (61)..(61): at position 61, “n” is either A or G
atcatgtttagtgtgtttggttgttcaaaaagatggagtcgcggtgcggccatgaactcgntttcttgggtttattattattattattagtttattactattaattactgctacatatatg

SEQ ID NO: 611 - DNA, 121 bp, Ocimum basilicum
  variation (61)..(61): at position 61, “n” is either A or T
gagataaggattgcaattttatgactttgtgagagaattatggttgtaattttataaattnaatgttgattttttaatttaatttaaatgtgagatgacgtggatacttaatgatcatatc

SEQ ID NO: 612 - DNA, 121 bp, Ocimum basilicum
  variation (61)..(61): at position 61, “n” is either A or C
atcaaagaaaaaagttctaccaagaaaatcgagagctttcgggcgaataaattacactaangaaaggcgagtgatgttggttagtgacccgggaatctctccagagagactgttgttgttg

SEQ ID NO: 613 - DNA, 121 bp, Ocimum basilicum
  variation (61)..(61): at position 61, “n” is either A or G
cagagttccttcccaggtaatgaccctgcaaaatagatttagtatagttaaggtgagatcngagatgcctgaactttaaagttgtaatgtggacaaatacatttctggagcatccaaactc

SEQ ID NO: 614 - DNA, 121 bp, Ocimum basilicum
  variation (61)..(61): at position 61, “n” is either T or C
taaatctcacaaaatcctctttctgactgatttttaggtctaaagatttctttttcccaanatttgaatcttttgtggaattgattttagtgaagaatatgttcgtaattgaggtttatct

SEQ ID NO: 615 - DNA, 121 bp, Ocimum basilicum
  variation (61)..(61): at position 61, “n” is either T or C
cctctttctgactgatttttaggtctaaagatttctttttcccaatatttgaatcttttgnggaattgattttagtgaagaatatgttcgtaattgaggtttatcttccgcggtcttagcg

SEQ ID NO: 616 - DNA, 121 bp, Ocimum basilicum
  variation (61)..(61): at position 61, “n” is either G or C
acaaaagcggtaaatccgcccccccttcttccacaaagcggaaatccaggtgattgtgcgncaacaaacttttactaatttctcctctttttactgcccggtgaaaacaattaggatcgtg

SEQ ID NO: 617 - DNA, 121 bp, Ocimum basilicum
  variation (61)..(61): at position 61, “n” is either A or T
aaaagcggtaaatccgcccccccttcttccacaaagcggaaatccaggtgattgtgcggcnacaaacttttactaatttctcctctttttactgcccggtgaaaacaattaggatcgtgct

SEQ ID NO: 618 - DNA, 121 bp, Ocimum basilicum
  variation (61)..(61): at position 61, “n” is either A or G
cgctctggatgcgtcagcaacgatgcgctgaacgttttgcggcgtcggacggaggaagtanacggcggggacgtcgtggacgggattccgatccttatcgacgaggaaatacagcgtgatt

SEQ ID NO: 619 - DNA, 121 bp, Ocimum basilicum
  variation (61)..(61): at position 61, “n” is either A or G
atcttatatggtgataaacattttttcttgtacatatgttggtttaataattttgtttctntataggttgacgttccaattaatgaacaaagtaatttttgtcagtattcaaatgcagtcc

SEQ ID NO: 620 - DNA, 121 bp, Ocimum basilicum
  variation (61)..(61): at position 61, “n” is either A or G
attaagcatatctacaaaatatatactgaccggaacgtgggaatctctggcgctgcgtttngcgtcggtggcggagaagacggtgtagaccagcacgaatgtgcccacgatttcagcaccg

SEQ ID NO: 621 - DNA, 121 bp, Ocimum basilicum
  variation (61)..(61): at position 61, “n” is either A or G
atccatgaaatatttgtcggaatgcagccacgtctccaagcttccattcggcatgtacgcnagaatcaaggccttaaaatcggtgttcatgttactgcaacaactaagaaccctgacaaga

SEQ ID NO: 622 - DNA, 121 bp, Ocimum basilicum
  variation (61)..(61): at position 61, “n” is either A or G
ctccatatttaacattgttggtaagtcatccaacgaaagatgacgtgactattatatattntgaagaaacgactgcaattgaatcgaagagataaaaagtcaaaaacttgtgaaagaaact

SEQ ID NO: 623 - DNA, 121 bp, Ocimum basilicum
  variation (61)..(61): at position 61, “n” is either G or A
cctaataagctccttctgaagagcgccgccctgcttagttctccggtgagaagtggccccnaagccggtggtgacaccgtagctgtcggttcctttgttcatactctccatgacccaatcg

SEQ ID NO: 624 - DNA, 121 bp, Ocimum basilicum
  variation (61)..(61): at position 61, “n” is either C or T
tccggcccatccgaccatggatatctcggcattttcactccatgcctaaaatcaacatatnctattagataattgcggtatatatgatgtgtactagtttatcttaagagtgtatttgttt

SEQ ID NO: 625 - DNA, 121 bp, Ocimum basilicum
  variation (61)..(61): at position 61, “n” is either G or A
atccatgtcggtgagagtaatgccaagattggtgagagttttcactatggagccagccatntcaggcatcagctgcagatcatcaccgattatgaaagaacctgatcgatgagttaaaatc

SEQ ID NO: 626 - DNA, 121 bp, Ocimum basilicum
  variation (61)..(61): at position 61, “n” is either C or T
ccatgtcggtgagagtaatgccaagattggtgagagttttcactatggagccagccatgtnaggcatcagctgcagatcatcaccgattatgaaagaacctgatcgatgagttaaaatccc

SEQ ID NO: 627 - DNA, 121 bp, Ocimum basilicum
  variation (61)..(61): at position 61, “n” is either A or G
cagggcgagaaaacaatcagccctgccacggcattacacctccgtctcaagtggaagttcnacgctggaaatgacgtaacagcaacgcctgccatctacaacggcattctctacttcccaa

SEQ ID NO: 628 - DNA, 121 bp, Ocimum basilicum
  variation (61)..(61): at position 61, “n” is either C or G
gattatgagatgggaaggtcagaaaccgacaggaattggagaagagtgtcgattgagaggnggagcatgatttaaggtgggaatttgtcggggtggtgagagtgtggtcggagagggagga

SEQ ID NO: 629 - DNA, 121 bp, Ocimum basilicum
  variation (61)..(61): at position 61, “n” is either C or T
tgatctgaccaatcaagagtatcggagaatgtatcttggcactagaccacgcggtgcccgncgcttgtccgccgccaagagcgatcgatatgcgctcagagacggggactccttgccggaa

SEQ ID NO: 630 - DNA, 121 bp, Ocimum basilicum
  variation (61)..(61): at position 61, “n” is either C or A
ttttgcaaccttatatttgtcaccttcgggagcttttgaaaaattaaggacaccattaccnaagattatatgctgtgtgttcggtaaacataaaagactctggctagtaaagctaacagta